(19) United States
(12) Patent Application Publication
(10) Pub. No.: US 2021/0395841 A1
(43) Pub. Date: Dec. 23, 2021

(54) DETECTION AND CLASSIFICATION OF HUMAN PAPILLOMAVIRUS ASSOCIATED CANCERS

(71) Applicant: GRAIL, Inc., Menlo Park, CA (US)

(72) Inventors: Robert Abe Paine Calef, Redwood City, CA (US); M. Cyrus Maher, San Mateo, CA (US); John F. Beausang, Menlo Park, CA (US); Joerg Bredno, San Francisco, CA (US); Oliver Claude Venn, San Francisco, CA (US); Alexander P. Fields, Burlingame, CA (US); Arash Jamshidi, Redwood City, CA (US)

(73) Assignee: GRAIL, Inc., Menlo Park, CA (US)

(21) Appl. No.: 17/350,511

(22) Filed: Jun. 17, 2021

Related U.S. Application Data
(60) Provisional application No. 63/041,875, filed on Jun. 20, 2020.

(51) Int. Cl. C12Q 1/70; C12Q 1/6886

(52) U.S. Cl. CPC C12Q 1/708; C12Q 2600/154; C12Q 1/6886

(57) ABSTRACT

Systems and methods described herein include detecting a presence or absence of HPV in a biological sample having cell-free nucleic acids from a subject and potentially cell-free nucleic acids from an HPV strain. Based on a detection of HPV viral nucleic acids in the biological sample, an HPV-based multiclass classifier that predicts a score for each HPV-associated cancer type is applied. The HPV-based multiclass classifier is trained on a training set of HPV-positive cancer samples. An HPV-associated cancer associated with the biological sample is determined based on the scores predicted by the HPV multiclass classifier.

### CROSS REFERENCE TO RELATED APPLICATIONS

This application claims the benefit of priority to U.S. Provisional Application No. 63/041,875, filed Jun. 20, 2020 and entitled “Detection and Classification of Human Papillomavirus Associated Cancers,” the contents of which are incorporated herein by reference in its entirety for all purposes.

### BACKGROUND OF THE INVENTION

Some cancers are known to be associated with a Human Papillomavirus (HPV) infection, such as anorectal, cervical, vulva, penile, and certain subtypes of head and neck cancers. Early detection and classification of HPV cancers (HPV-associated cancers) can lead to earlier treatment, and thus, lower mortality associated with HPV-associated cancers. Accordingly, there is a need in the art for improved methods for the detection and classification of HPV-associated cancers.

### SUMMARY OF THE INVENTION

### Field of the Invention

The present disclosure relates generally to cancer detection, and more specifically to cancer detection using detection (e.g., via sequencing) of Human papillomavirus (HPV) in a biological sample.

In some aspects, a method of screening for detecting an HPV-associated cancer in a subject comprises: (a) obtaining a biological sample from the test subject, wherein the biological sample comprises cell-free nucleic acids from the test subject and potentially cell-free nucleic acids from at least one HPV strain; (b) sequencing the cell-free nucleic acid in the first biological sample to generate a plurality of sequence reads from the test subject; (c) determining an amount of the plurality of sequence reads that map to one or more HPV reference genomes corresponding to one or more HPV strains, wherein the amount comprises a count of unique sequence reads that map to the one or more HPV reference genomes; and (d) detecting an HPV-associated cancer in the subject when the amount of unique sequence reads exceeds a cutoff.

Various embodiments are contemplated in the present invention. For instance, in some embodiments, the amount of unique sequence reads comprises a total count of unique sequence reads that map to one or more HPV reference genomes corresponding to the one or more HPV strains. In some embodiments, the one or more HPV strains includes HPV 16 and/or HPV 18. In some examples, the one or more HPV strains include one or more of HPV 16, 18, 31, 33, 35, 39, 45, 51, 52, 56, 58, 59, 66 and 68.

In some embodiments, sequencing comprises whole genome sequencing, targeted sequencing, or whole genome bisulfite sequencing. In some embodiments, the HPV-associated cancer comprises at least one of cervical, anogenital, and head and neck cancers. In some embodiments, the cutoff is more than 5 unique sequence reads, more than 10 unique sequence reads, and/or more than 20 unique sequence reads. In some embodiments, the cutoff is a cross-validated HPV DNA fragment count cutoff associated with a target specificity for detecting HPV-associated cancers. In some embodiments, the target specificity is within the range of 99.0-99.9%.

In some aspects, a method of screening for presence of an HPV-associated cancer in a subject comprises: detecting a presence or absence of HPV in a biological sample comprising cell-free nucleic acids from the subject and potentially cell-free nucleic acids from at least one HPV strain in a set of HPV strains; based on a detection of HPV viral nucleic acids in the biological sample, applying an HPV-based multiclass classifier that predicts a score for each of a plurality of HPV-associated cancer types, wherein the HPV-based multiclass classifier is trained on a training set comprising HPV-positive cancer samples; and determining, based on the scores predicted by the HPV multiclass classifier, an HPV-associated cancer associated with the biological sample.

Various embodiments are contemplated in the present invention. For instance, in some embodiments, detecting the presence or absence of HPV viral nucleic acids in the biological sample comprises: determining an amount of HPV fragments in the biological sample that are derived from the potentially cell-free nucleic acid from the at least one HPV strain in the set of HPV strains; comparing the amount of HPV fragments to a cutoff, and detecting HPV presence in the biological sample when the amount exceeds the cutoff.

In some embodiments, determining the amount of HPV fragments comprises: sequencing the cell-free nucleic acids and potentially cell-free nucleic acids from one or more HPV strains to obtain a plurality of sequence reads; and determining the amount of HPV fragments based on a total count of the plurality of sequence reads that map to one or more HPV reference genomes corresponding to the one or more HPV strains.

In some embodiments, the sequencing is performed by whole genome sequencing, targeted sequencing, or whole genome bisulfite sequencing.

In some embodiments, the cutoff is a count of at least 6 unique HPV fragments, each unique HPV fragment mapping to an HPV reference genome corresponding to at least one HPV strain in the set of HPV strains.

In some embodiments, the set of HPV strains comprises at least one of HPV 16 or HPV 18. In some examples, the set of HPV strains includes one or more of HPV 16, 18, 31, 33, 35, 39, 45, 51, 52, 56, 58, 59, 66 and 68.

In some embodiments, the HPV-based multiclass classifier predicts the scores based on features derived from sequencing the potentially cell-free nucleic acid from the at least one HPV strain in a set of HPV strains in the biological sample, wherein the features comprise one or more of methylation-derived features, a total count of HPV fragments, and a binarized count of HPV fragments. In some embodiments, the methylation-derived features comprise features that discriminate pairwise comparisons among HPV-associated cancer types and other cancer types, wherein the other cancer types comprise lung cancers.

In some embodiments, the sequencing is performed by whole genome sequencing, targeted sequencing, or whole genome bisulfite sequencing. In some embodiments, the sequencing is performed by targeted sequencing with a hybridization capture panel containing probes targeting HPV reference genomes corresponding to the set of HPV strains. In some embodiments, the probes tile the targeted HPV reference genomes.

In some embodiments, the plurality of HPV-associated cancer types comprise cervical, anogenital, and head and neck cancers.

In some embodiments, the HPV-based multiclass classifier comprises a multinomial logistic regression classifier. In some embodiments, training of the HPV-based multiclass classifier is restricted to the HPV-positive cancer samples, wherein the HPV-positive cancer samples comprise at least one of cervical, anorectal, and head and neck cancers.

In some embodiments, the method includes, based on a detection of HPV absence from the biological sample: forgoing applying the HPV-based multiclass classifier, or determining an absence of HPV-associated cancer from the biological sample.

In some aspects, a method of predicting a presence or absence of cancer in a test sample containing cell-free nucleic acids, the cell-free nucleic acids comprising cell-free nucleic acids from a test subject and potentially cell-free nucleic acids from at least one HPV strain, comprises: accessing the test sample having a first cancer type, wherein the first cancer type is determined by a first multiclass classifier that generates, based on a set of features derived from sequencing the cell-free nucleic acids in the test sample, an initial score for the first cancer type; in accordance with a determination that the first cancer type is an HPV-associated cancer type: applying a second multiclass classifier to the set of features to determine a a second score corresponding to a second cancer type, wherein the second multiclass classifier is trained only on HPV-positive cancer samples; and determining a level of cancer for the test sample based on the second cancer type, wherein the level of cancer comprises a presence or absence of cancer, a cancer type, or a cancer tissue of origin.

Various embodiments are contemplated in the present invention. For instance, in some embodiments, the HPV-associated cancer type comprises cervical, anogenital, or head and neck cancer. In some embodiments, features in the set of features comprise one or more methylation-derived features, a total count of HPV fragments or a binarized count of HPV fragments, and/or an HPV signal status. In some embodiments, the total count of HPV fragments and the binarized count of HPV fragments comprise a quantified count of unique sequence reads mapping to HPV 16 and/or HPV 18 reference genomes.

In some embodiments, the HPV signal status comprises an HPV-positive signal status defined by a presence of HPV cell-free nucleic acid fragments or an HPV-negative signal status defined by an absence of HPV cell-free nucleic acid fragments, further wherein presence of the HPV cell-free nucleic acid fragments is confirmed when a quantification of unique sequence reads mapping to HPV 16 and HPV 18 reference genomes is greater than a threshold.

In some embodiments, the threshold is 6 unique sequence reads mapping to HPV 16 and/or HPV 18 reference genomes. In some embodiments, the sequencing is performed by whole genome sequencing, targeted sequencing, or whole genome bisulfite sequencing. In some embodiments, the sequencing comprises a targeted pulldown of HPV 16 and HPV 18 nucleic acid sequences in the cell-free nucleic acid in the test sample.

In some embodiments, the first multiclass classifier comprises a plurality of classes corresponding to a plurality of HPV-associated cancer types and non-HPV-associated cancer types. In some embodiments, the second multiclass classifier comprises at least three classes corresponding to three HPV-associated cancer types, including cervical, anogenital, and head and neck cancers.

In some embodiments, the first multiclass classifier is trained using a set of training features derived from a plurality of HPV-associated cancer type samples and non-HPV-associated cancer type samples, the set of training features including methylation-derived features, and wherein the second multiclass classifier is trained using a restricted set of training features from the set of training features, the restricted set of training features being restricted to features derived from the plurality of HPV-associated cancer type samples.

In some embodiments, the method includes, in accordance with a determination that the first cancer type is not an HPV-associated cancer type, forgoing applying the second multiclass classifier to the set of features; and determining a level of cancer for the test sample based on the first cancer type, wherein the level of cancer comprises a presence or absence of cancer, a cancer type, or a cancer tissue of origin.

In some embodiments, the total count of HPV fragments or the binarized count of HPV fragments comprise a quantified count of unique sequence reads mapping to one or more HPV reference genomes. In some embodiments, the HPV signal status comprises an HPV-positive signal status defined by a presence of HPV cell-free nucleic acid fragments or an HPV-negative signal status defined by an absence of HPV cell-free nucleic acid fragments, further wherein presence of the HPV cell-free nucleic acid fragments is confirmed when a quantification of unique sequence reads mapping to one or more HPV reference genomes is greater than a threshold. In some embodiments, the threshold is 6 unique sequence reads mapping to one or more HPV reference genomes. In some embodiments, the HPV reference genomes are associated with one or more strains of HPV 16, 18, 31, 33, 35, 39, 45, 51, 52, 56, 58, 59, 66 and 68.

In some aspects, a method for detecting and classifying cancer, comprises: receiving sequencing data for a biological sample comprising cell-free nucleic acid fragments; deriving a set of features from the sequencing data, wherein the set of features comprises methylation-derived features and at least one of: a total count of HPV fragments, a binarized count of HPV fragments, or an HPV signal status; applying a multiclass classifier to the set of features, wherein the multiclass classifier predicts a probability likelihood for each of a plurality of cancer types, wherein the plurality of cancer types comprises HPV-associated cancer types and non-HPV-associated cancer types; and determining a cancer classification based on the probability likelihoods, wherein the cancer classification comprises a presence or absence of cancer, a cancer type, a cancer tissue of origin, a presence or absence of an HPV-associated cancer, an HPV-associated cancer type, or an HPV-associated cancer tissue of origin.

Various embodiments are contemplated in the present invention, such as any of the numerous variations and examples described above and further herein.

In some aspects, a method of detecting a level of cancer in a test sample comprising cell-free nucleic acids from a test subject and potentially cell-free nucleic acids from an HPV strain, comprises: obtaining sequencing data generated by sequencing the cell-free nucleic acids; generating a first set of features based on methylation status at one or more CpG sites observed in the sequencing data; generating at least one second feature based on a count of HPV-derived sequence reads in the sequencing data; applying a first multiclass classifier to the first set of features and the at least one second feature to determine a first cancer classification, wherein the multiclass classifier is trained on training samples corresponding to positive cancer samples, the positive samples including HPV-associated cancer types and non-HPV-associated cancer types; in accordance with a determination that the first cancer classification corresponds to an HPV-associated cancer type: applying a second multiclass classifier to the first set of features and the at least one second feature to determine a second cancer classification, wherein the second multiclass classifier is trained only on positive cancer samples having HPV-associated cancer types; and determining a level of cancer based on the first cancer classification and/or the second cancer classification.

Various are contemplated in the present invention, such as any of the numerous variations and examples described above and further herein.

In various embodiments, a system comprises a computer processor and a memory, the memory storing computer program instructions that when executed by the computer processor cause the processor to perform any of the methods described herein. In various embodiments, a non-transitory computer-readable medium stores one or more programs, the one or more programs including instructions which, when executed by an electronic device including a processor, cause the device to perform any of the methods described herein.

### BRIEF DESCRIPTION OF THE DRAWINGS

The implementations disclosed herein are illustrated by way of example, and not by way of limitation, in the figures of the accompanying drawings. Like reference numerals refer to corresponding parts throughout the several views of the drawings.

FIG. 1 is a flowchart of a method for generating a classifier to predict disease state, according to various embodiments.

FIG. 2A illustrates a flowchart of devices for sequencing nucleic acid samples according to one embodiment.

FIG. 2B is a block diagram of an analytics system for processing sequence reads, according to various embodiments.

FIG. 3 is a flowchart describing a process of sequencing nucleic acids, according to various embodiments.

FIG. 4A is an illustration of a part of the process of FIG. 3 of sequencing nucleic acids to obtain methylation information and methylation state vectors, according to various embodiments.

FIG. 4B illustrates generation of a data structure for a control group, according to various embodiments.

FIG. 4C illustrates a flowchart describing a process of determining anomalously methylated fragments from a sample, according to various embodiments.

FIG. 5 is an illustration of blocks of a reference genome, according to various embodiments.

FIG. 6 is an illustration of a process of determining features to train a classifier, according to various embodiments.

FIG. 7A includes confusion matrices indicating the performance of classifiers based on various models, according to various embodiments.

FIG. 7B includes confusion matrices indicating the performance of classifiers trained on different training sets, according to various embodiments.

FIG. 7C includes further confusion matrices indicating the performance of classifiers trained on different training sets, according to various embodiments.

FIG. 8 is a flowchart of a method for model-based featurization, according to various embodiments.

FIG. 9A illustrates the sensitivity of a tissue of origin classifier for a group of cancers, according to various embodiments.

FIG. 9B illustrates the sensitivity of a tissue of origin classifier for another group of cancers, according to various embodiments.

FIG. 10A illustrates the sensitivity of a tissue of origin classifiers at different cancer stages, according to various embodiments.

FIG. 10B further illustrates the sensitivity of a tissue of origin classifier at different cancer stages, according to various embodiments.

FIG. 11 illustrates a performance grid representing the accuracy of tissue of origin localization, according to various embodiments.

FIG. 12A illustrates a graph of HPV fragment count versus fraction of samples, according to various embodiments.

FIG. 12B illustrates various bar charts comparing HPV fragment counts across various cancer type classes, according to various embodiments.

FIG. 13A illustrates a bar chart showing HPV 16 and HPV 18 fragment counts in cfDNA samples for various cancer types, according to various embodiments.

FIG. 13B illustrates a bar chart showing HPV 16 and HPV 18 fragment counts in tissue samples for various cancer types, according to various embodiments.

FIG. 13C illustrates a bar chart showing HPV fragment counts across different HPV statuses, according to various embodiments.

FIG. 13D illustrates a bar chart showing HPV fragment counts by tumor type across different cancer samples, according to various embodiments.

FIG. 13E illustrates a bar chart showing head/neck HPV fragment count by tumor location, according to various embodiments.

FIG. 14 illustrates a graph demonstrating that some currently undetected cancers are above certain specificity threshold cutoffs, according to various embodiments.

FIG. 15A illustrates a UMAP embedding of features from a training set for all samples, according to various embodiments.

FIG. 15B illustrates a UMAP embedding of features from a training set for evaluation samples, according to various embodiments.

FIG. 15C illustrates a UMAP embedding of selective features from a training set for all samples, according to various embodiments.

FIG. 15D illustrates UMAP embedding of selective features from a training set for evaluation samples, according to various embodiments.

FIG. 16 illustrates various plots showing head and neck feature bias towards HPV positive patients, according to various embodiments.

FIG. 17A illustrates various plots representing a reduction of head and neck feature bias, according to various embodiments.

FIG. 17B illustrates further plots representing the reduction of head and neck features bias, according to various embodiments.

FIG. 18A illustrates a UMAP embedding of features from a train set for all samples, after the reduction of head and neck feature bias, according to various embodiments.

FIG. 18B illustrates a UMAP embedding of features from a train set for evaluation samples, after the reduction of head and neck feature bias, according to various embodiments.

FIG. 19A illustrates a confusion matrix showing classification results of a multiclass classifier, according to various embodiments.

FIG. 19B illustrates a confusion matrix showing classification results of an HPV-based multiclass classifier, according to various embodiments.

FIG. 19C illustrates a confusion matrix showing classification results of another HPV-based multiclass classifier, according to various embodiments.

FIG. 20A illustrates a bar chart showing HPV DNA fragment counts by clinically diagnosed HPV status, according to various embodiments.

FIG. 20B illustrates a bar chart showing HPV 16 versus HPV 18 DNA fragment counts in tumor biopsies by tissue type, according to various embodiments.

FIG. 20C illustrates a bar chart showing HPV DNA fragment counts in head and neck cancer participants by tumor location, according to various embodiments.

FIG. 20D illustrates a bar chart showing HPV DNA fragment counts in plasma cfDNA samples by cancer type, according to various embodiments.

FIG. 20E illustrates a UMAP embedding of detectable cancers of the anus, cervix, lung, and head and neck cancers, according to various embodiments.

FIG. 21 is a flowchart of an example method for screening for detecting an HPV-associated cancer in a subject, according to various embodiments.

FIG. 22 is a flowchart of an example method for screening for presence of an HPV-associated cancer in a subject, according to various embodiments.

FIG. 23 is a flowchart of an example method for predicting a presence or absence of cancer in a test sample containing cell-free nucleic acids, according to various embodiments.

FIG. 24 is a flowchart of an example method for detecting and classifying cancer, according to various embodiments.

FIG. 25 is a flowchart of an example method for detecting a level of cancer in a test sample comprising cell-free nucleic acids from a test subject and potentially cell-free nucleic acids from a HPV strain, according to various embodiments.

### DETAILED DESCRIPTION OF THE INVENTION

Reference will now be made in detail to several embodiments, examples of which are illustrated in the accompanying figures. It is noted that wherever practicable similar or like reference numbers may be used in the figures and may indicate similar or like functionality. It is also noted that the contents of all published materials (patent applications, patents, papers, conference proceedings, and the like) referenced herein are incorporated herein by reference in their entirety

### Definitions

Unless defined otherwise, all technical and scientific terms used herein have the meaning commonly understood by a person skilled in the art to which this description belongs. As used herein, the following terms have the meanings ascribed to them below.

The term “individual” refers to a human individual. The term “healthy individual” refers to an individual presumed to not have a cancer or disease.

The term “subject” refers to an individual whose DNA is being analyzed. A subject can be a test subject whose DNA is to be evaluated using whole genome sequencing or a targeted panel as described herein to evaluate whether the person has a disease state (e.g., cancer, type of cancer, or cancer tissue of origin). A subject can also be part of a control group known not to have cancer or another disease. A subject can also be part of a cancer or other disease group known to have cancer or another disease. Control and cancer/disease groups can be used to assist in designing or validating the targeted panel.

The term “reference sample” refers to a sample obtained from a subject with a known disease state.

The term “training sample” refers to a sample obtained from a known disease state that can be used to generate sequence reads. Training samples can be applied to probability models to generate features that can be utilized for disease state classification.

The term “test sample” refers to a sample that may have an unknown disease state.

The term “sequence read” refers to a nucleotide sequence read from a sample obtained from an individual. Sequence reads can be generated from nucleic acid fragments in the sample. A sequence read can be a collapsed sequence read generated from a plurality of sequence reads derived from a plurality of amplicons from a single original nucleic acid molecule. In some embodiments, the sequence read can be a deduplicated sequence read. Sequence reads can be obtained through various methods known in the art.

The term “disease state” refers to presence or non-presence of a disease, a type of disease, and/or a disease tissue of origin. For example, in one embodiment, the present disclosure provides methods, systems, and non-transitory computer readable medium for detecting cancer (i.e., presence or absence of cancer), a type of cancer, or a cancer tissue of origin.

The term “tissue of origin” or “TOO” refers to the organ, organ group, body region or cell type from which a disease state can arise or originate. For example, the identification of a tissue of origin or cancer cell type typically allows the identification of appropriate next steps to further diagnose, stage, and decide on treatment. In some cases, tissue of origin or TOO is used interchangeably with “cancer signal origin” or “CSO”.

The term “methylation” as used herein refers to a chemical process by which a methyl group is added to a DNA molecule. Two of DNA's four bases, cytosine (“C”) and adenine (“A”) can be methylated. For example, a hydrogen atom on the pyrimidine ring of a cytosine base can be converted to a methyl group, forming 5-methylcytosine. Methylation tends to occur at dinucleotides of cytosine and guanine referred to herein as “CpG sites.” In other instances, methylation can occur at a cytosine not part of a CpG site or at another nucleotide that is not cytosine; however, these are rarer occurrences. In this present disclosure, methylation is discussed in reference to CpG sites for the sake of clarity. However, the principles described herein are equally applicable for the detection of methylation in a non-CpG context, including non-cytosine methylation. For example, Adenine methylation has been observed in bacteria, plant and mammalian DNA, although it has received considerably less attention.

In such embodiments, the wet laboratory assay used to detect methylation can vary from those described herein as well known in the art. Further, the methylation state vectors can contain elements that are generally vectors of sites where methylation has or has not occurred (even if those sites are not CpG sites specifically). With that substitution, the remainder of the processes described herein are the same, and consequently the inventive concepts described herein are applicable to those other forms of methylation.

The term “CpG site” refers to a region of a DNA molecule where a cytosine nucleotide is followed by a guanine nucleotide in the linear sequence of bases along its 5′ to 3′ direction. “CpG” is a shorthand for 5′-C-phosphate-G-3′ that is cytosine and guanine separated by only one phosphate group; phosphate links any two nucleotides together in DNA. Cytosines in CpG dinucleotides can be methylated to form 5-methylcytosine.

The term “methylation site” refers to a single site of a DNA molecule where a methyl group can be added. “CpG” sites are the most common methylation site, but methylation sites are not limited to CpG sites. For example, DNA methylation may occur in cytosines in CHG and CHH, where H is adenine, cytosine or thymine. Cytosine methylation in the form of 5-hydroxymethylcytosine can also assessed (see, e.g., WO 2010/037001 and WO 2011/127136, which are incorporated herein by reference in their entirety), and features thereof, using the methods and procedures disclosed herein. The term “hypomethylated” or “hypermethylated” refers to a methylation status of a DNA molecule containing multiple CpG sites (e.g., more than 3, 4, 5, 6, 7, 8, 9, 10, etc.) where a high percentage of the CpG sites (e.g., more than 80%, 85%, 90%, or 95%, or any other percentage within the range of 50%-100%) are unmethylated (hypomethylated) or methylated (hypermethylated), respectively.

The term “cell free deoxyribonucleic nucleic acid,” “cell free DNA,” or “cfDNA” refers to deoxyribonucleic acid fragments that circulate in bodily fluids such as blood, sweat, urine, or saliva and originate from one or more healthy cells and/or from one or more cancer cells.

The term “circulating tumor DNA” or “ctDNA” refers to deoxyribonucleic acid fragments that originate from tumor cells or other types of cancer cells, which can be released into an individual's bodily fluids such as blood, sweat, urine, or saliva as result of biological processes such as apoptosis or necrosis of dying cells or actively released by viable tumor cells

### Detection of Viral Cell-free Nucleic Acid Molecules

As described in more detail herein, in some embodiments, viral cell-free nucleic acid molecules are detected and evaluated in generating cancer classifications, such as for detecting a level of cancer or determining a cancer type from a biological sample from a subject. Examples of systems and methods for pathogen analysis during cancer classification are described herein and further in, for example, International Pat. App. No. PCT/US2019/028916, entitled “Systems and Methods for Using Pathogen Nucleic Acid Load to Determine Whether a Subject Has a Cancer Condition,” and filed on Apr. 24, 2019, and International Pat. App. No. PCT/CN2018/097072, entitled “Enhancement of Cancer Screening Using Cell-free Viral Nucleic Acids” and filed on Jul. 25, 2018, the contents of which are incorporated herein by reference to their entirety.

### Detection of Pathogen Load

Some aspects of the present disclosure provide methods of screening for a cancer condition in a test subject based on genetic material that is derived from one or more pathogens, such as human papillomavirus (HPV), and in some examples particularly the most cancer-causing HPV types. For instance, a method can include obtaining a first biological sample from the test subject. The first biological sample comprises cell-free nucleic acid from the test subject and potentially cell-free nucleic acid from at least one pathogen in a set of pathogens, such as at least one HPV strain in a set of HPV strains. Such HPV strains can include HPV 16 and/or HPV 18. In some examples, such HPV strains include strains that can be considered the most cancer-causing, such as any of the following HPV strains: 16, 18, 31, 33, 35, 39, 45, 51, 52, 56, 58, 59, 66 and 68.

In some embodiments, the cell-free nucleic acid in the first biological sample can be sequenced (e.g., by whole genome sequencing, targeted panel sequencing, or whole genome bisulfite sequencing, etc.) to generate a plurality of sequence reads from the test subject and HPV-derived fragments can be detected therefrom. In other embodiments, HPV-derived fragments (or HPV sequences) can be detected (e.g., as an HPV fragment or HPV sequence read) using amplification based detection means, such as, detection by polymerase chain reaction (PCR), digital PCR (dPCR), quantitative PCR (qPCR), real time PCR (RT-PCR), quantitative real time PCR (qRT-PCR), or other well-known means in the art. For each respective pathogen (e.g., HPV fragment) in the set of pathogens (e.g., HPV strains), a corresponding amount of the plurality of HPV fragments or sequence reads that map to a pathogen target reference, such as an HPV reference genome, for the respective pathogen can be determined, thereby obtaining an amount of HPV fragments, or in some cases, a total count of unique sequence reads across multiple HPV reference genomes (e.g., a total count of sequence reads mapping to HPV 16 and HPV 18 reference genomes). The amount of sequence reads can be used to determine whether the test subject has a cancer condition, such as a likelihood that the test subject has the cancer condition. Such cancer conditions can be, for example, a level of cancer and/or a cancer type, such as an HPV-driven cancer type which can include, by way of example, anorectal, cervical, vulva, penile, and certain subtypes of head and neck cancers.

It will be appreciated that a pathogen reference genome (e.g., an HPV reference genome) can include several different reference genomes (e.g., from several different HPV strains) or several different regions from one or more pathogen reference genomes. In some examples, a sequence read from the test subject need only map onto one of these reference genomes in order to counted as a pathogen sequence (e.g., HPV) mapping to the pathogen target reference. Thus, a first sequence read from the test subject that maps to a first reference genome or to a first region of the pathogen reference genome will contribute to the amount of sequence reads that map onto the pathogen reference genome, as will a second sequence read from the test subject that maps to a second reference genome or to a second region of the pathogen reference genome. Whereas if a third sequence read from the test subject does not map onto any of the several different reference genomes or to any of the several different regions from one or more of the pathogen reference genomes, then that third sequence read will not contribute to the amount of sequence reads that map onto the pathogen reference genome.

In some examples, the method relies upon a panel (i.e., a targeted viral panel) comprising several targeted regions from one or more pathogen genomes (e.g., one or more HPV genomes). For example, in such embodiments, the targeted panel can include enrichment probes, to enrich and pulldown DNA molecules derived from one or more HPV strains (e.g., HPV-16 and/or HPV-18). In some examples, the targeted panel (e.g., a targeted HPV panel) for a particular pathogen is limited to a minimum or maximum number of regions from the pathogen, such as 100 regions or less, 50 regions or less, or 25 regions or less. In some examples, as described herein, such thresholds can be determined based on a desired panel size and available space thereof.

In some examples, the pathogen reference genome includes a set of pathogen reference genomes, and the sequence reads from a sample are pooled together and mapped to each of the pathogen reference genomes. In some such examples, separate counts can be used to track sequence reads that map to each of the pathogen reference genomes.

In some examples, the mapping of sequence reads from the test subject to a sequence in a HPV reference genome for a respective HPV strain comprises a sequence alignment between (i) one or more sequence reads in the plurality of sequence reads (from the test subject) and (ii) a sequence in the HPV reference genome for the respective HPV pathogen.

In some examples, the mapping of sequence reads from the test subject to a sequence in a HPV reference genome for a respective pathogen comprises a comparison of a methylation-derived characteristic or feature between (i) a sequence read in one or more of the plurality of sequence reads and (ii) a sequence in the HPV reference genome for the respective HPV pathogen.

In some examples, the method relies upon whole genome sequencing. In some such examples, the pathogen reference genome comprises a HPV reference genome for each HPV strain in a set of HPV strains. Then, for each respective HPV strain in the set of HPV strains, a corresponding amount of the plurality of sequence reads that map to a sequence in each of the respective HPV genomes is determined. Such alignment can be performed by aligning each sequence read in the plurality of sequence reads using the entire reference genome of the respective pathogen or to a limited set of regions from each of the respective pathogens.

In some examples, the HPV reference genome for a respective HPV strain includes at least a portion of the reference genome of the respective HPV strain (e.g., less than 10 percent of the reference genome, less than 25 percent of the reference genome, less than 50 percent of the reference genome, less than 90 percent of the reference genome, or between 10 percent than 90 percent of the reference genome etc.). In some cases, alignment can be performed by aligning each sequence read in the plurality of sequence reads using the entire reference genome of the respective pathogen or to a portion of the reference genome.

In some examples, the method relies upon whole genome bisulfite sequencing. In such examples, methods can include, for each respective HPV strain in the set of HPV strains, a corresponding amount of the plurality of sequence reads that map to a sequence in each of the HPV reference genomes. In some embodiments, methods can include determining, for the respective HPV strain, a methylation-derived characteristic or feature related to one or more sequence reads in the plurality of sequence reads.

In some examples, the set of HPV strains is a single HPV strain. In alternative examples, the set of HPV strains is a plurality of HPV strains, and determining a corresponding amount of the plurality of sequence reads that map to a sequence in a HPV reference genome is performed for each respective HPV strain in the plurality of HPV strains. In some examples, the set of HPV strains comprises between 200 and 500 HPV strains, between 2 and 50 HPV strains, between 2 and 30 HPV strains, or 2 HPV strains.

### Comparing an Amount Reflecting Pathogen Load to a Reference/Cutoff Value.

In some examples, the use of the amount of sequence reads to determine whether the test subject has the cancer condition or the likelihood that the test subject has the cancer condition includes determining a cutoff or threshold amount of sequence reads for an HPV strain in the set of HPV strains, or determining a cutoff or threshold amount encompassing all of the HPV strains in the set of HPV strains. In such examples, a quantification of the amount of the plurality of sequence reads that map to the HPV reference genome(s) for the HPV strain(s) from the test subject can be compared to the cutoff to determine a level of cancer and/or cancer type. For instance, in some examples, if a total count of sequence reads mapping to the HPV reference genome(s) exceeds the cutoff or threshold, the test subject can be deemed to have or likely to have an HPV-derived cancer. Additionally and/or alternatively, in some examples, if the total count of sequence reads mapping to the HPV reference genome(s) exceeds the cutoff or threshold, the sequence reads and/or data thereof can be further analyzed by one or more specialist classifiers, such as an HPV-specific multiclass classifier. In some examples, such an HPV-specific multiclass classifier can be trained only on HPV-associated positive cancer samples, and can in some cases produce refined results in identifying and differentiating between HPV-driven cancer types.

### Overview of Methylation-Based Sequencing and Multiclass Classifiers

In some embodiments, the present systems and methods utilize methylation-based sequencing and data derived therefrom to produce cancer classifications using binary, or multiclass classifiers. Examples of systems and methods of methylation-based sequencing, featurization, classifiers, and performance are described herein and further in, for example, U.S. patent application Ser. No. 15/931,022, entitled “Model-based Featurization and Classification,” and filed on May 13, 2020, and International Pat. App. No. PCT/US2019/022122, entitled “Anomalous Fragment Detection and Classification,” and filed on Mar. 13, 2019, which are incorporated herein by reference to their entirety.

### Overview of Method

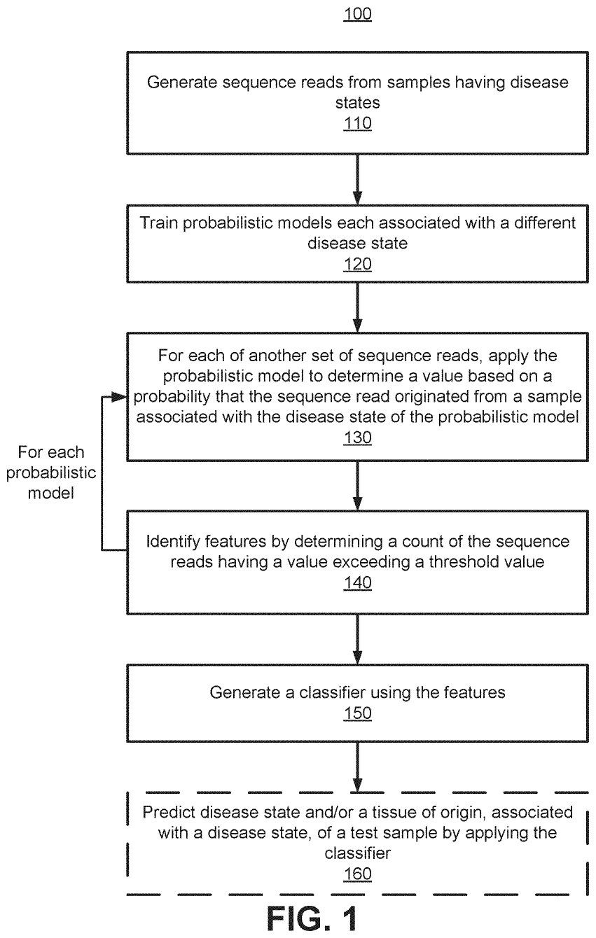

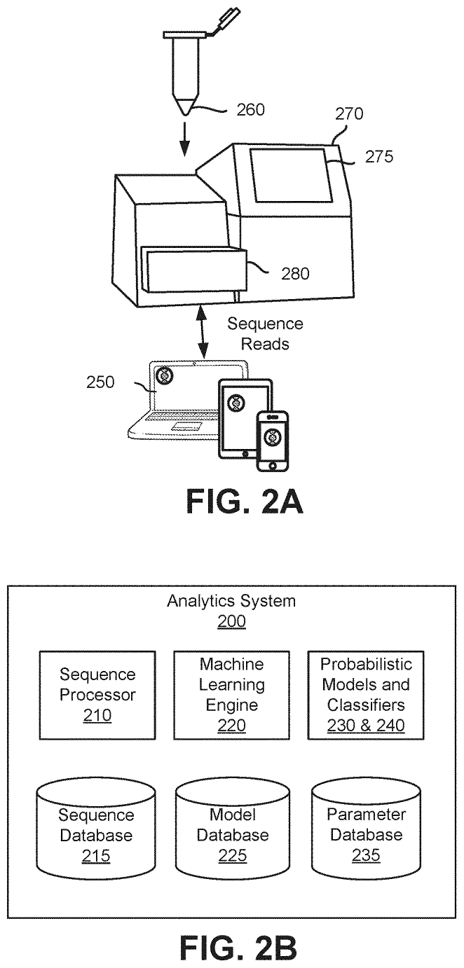

FIG. 1 is a flowchart of a method 100 for identifying a plurality of features for generating a classifier to predict a disease state (e.g., presence or absence of a disease, type of disease, and/or a disease tissue of origin), according to various embodiments. FIG. 2B is a block diagram of a processing or analytics system 200 for processing sequence reads, according to various embodiments. In some embodiments, the analytics system 200 performs the method 100 to process sequence reads of fragments from nucleic acid samples. The method 100 includes, but is not limited to, the following steps: generating sequence reads; training probabilistic models associated with each of a plurality of different disease states (e.g., different cancer types); applying the probabilistic models to determine a value based on a probability that a sequence read originated from a sample associated with each of the plurality of disease states associated with each probabilistic model; identifying features by determining a count of sequence reads having a value exceeding a threshold; generating a classifier using the features, and optionally applying the classifier to predicting disease state and/or a tissue of origin, associated with a disease state. Each of which are described with respect to the components of the analytics system 200 and with reference to FIGS. 2-6. In the embodiment shown in FIG. 2B, the analytics system 200 includes a sequence processor 210, a machine learning engine 220, probabilistic models 230, and a classifier 240.

In step 110, the sequence processor 210 generates a first set of sequence reads from a plurality of samples each having a known or suspected disease state, such as a presence or absence of a disease, a type of disease, and/or a disease tissue of origin. For example, in some embodiments, the plurality of samples can include any number of cancer samples from individuals known to have cancer and/or non-cancer samples from healthy individuals. Additionally, the samples can include any of cell free nucleic acid samples (e.g., cfDNA), solid tumor samples, and/or other types of samples. As one of skill in the art would appreciate, next generation sequencing procedures can generate a plurality of sequence reads from a single original nucleic acid molecule. Accordingly, in some embodiments, the sequence processor 210 can use known methods for deduplication and/or collapsing sequence reads to remove duplicate sequence reads and identify a single sequence read for a single original nucleic molecule from which one or more raw sequence reads were generated.

### Example Assay Protocol

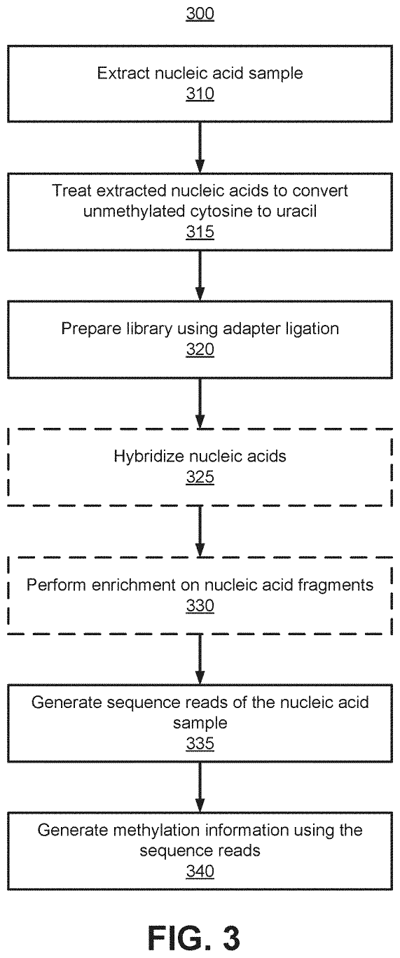

FIG. 3 is a flowchart describing a process 300 of sequencing nucleic acids, according to some embodiments. In some embodiments, the process 300 is performed to generate the sequence reads as part of step 110 of the method 100 of FIG. 1.

In step 310, a nucleic acid sample (e.g., DNA or RNA) is extracted from a subject. In the present disclosure, DNA and RNA can be used interchangeably unless otherwise indicated. That is, the embodiments described herein can be applicable to both DNA and RNA types of nucleic acid sequences. However, the examples described herein can focus on DNA for purposes of clarity and explanation. The sample can include nucleic acid molecules derived from any subset of the human genome, including the whole genome. The sample can include blood, plasma, serum, urine, fecal, saliva, other types of bodily fluids, or any combination thereof. In some embodiments, methods for drawing a blood sample (e.g., syringe or finger prick) can be less invasive than procedures for obtaining a tissue biopsy, which can require surgery. The extracted sample can comprise cfDNA and/or ctDNA. If a subject has a disease state, such as cancer, cell free nucleic acids (e.g., cfDNA) in an extracted sample from the subject generally includes a detectable level of the nucleic acids that can be used to assess a disease state.

In step 315, in some embodiments (e.g., where methylation-derived features are desired for subsequent analysis and classification), the extracted nucleic acids (e.g., including cfDNA fragments) are optionally treated to convert unmethylated cytosines to uracils. In other embodiments (e.g., where at least one HPV-derived feature is desired for subsequent analysis and classification), the extracted nucleic acids may, or may not, be treated to convert unmethylated cytosines to uracils. In some embodiments, the method 300 uses a bisulfite treatment of the samples which converts the unmethylated cytosines to uracils without converting the methylated cytosines. For example, a commercial kit such as the EZ DNA Methylation™—Gold, EZ DNA Methylation™—Direct or an EZ DNA Methylation™—Lightning kit (available from Zymo Research Corp (Irvine, Calif.)) is used for the bisulfite conversion. In another embodiment, the conversion of unmethylated cytosines to uracils is accomplished using an enzymatic reaction. For example, the conversion can use a commercially available kit for conversion of unmethylated cytosines to uracils, e.g., APOBEC-Seq (NEBiolabs, Ipswich, Mass.).

In step 320, a sequencing library is prepared. In some embodiments, the preparation includes at least two steps. In one exemplified sequencing library preparation method, in a first step, a ssDNA adapter can be added to the 3′-OH end of a bisulfite-converted ssDNA molecule using a ssDNA ligation reaction. In some embodiments, the ssDNA ligation reaction uses CircLigase II (Epicentre) to ligate the ssDNA adapter to the 3′-OH end of a bisulfite-converted ssDNA molecule, wherein the 5′-end of the adapter is phosphorylated and the bisulfite-converted ssDNA has been dephosphorylated (i.e., the 3′ end has a hydroxyl group). In another embodiment, the ssDNA ligation reaction uses Thermostable 5′ AppDNA/RNA ligase (available from New England BioLabs (Ipswich, Mass.)) to ligate the ssDNA adapter to the 3′-OH end of a bisulfite-converted ssDNA molecule. In this example, the first UMI adapter is adenylated at the 5′-end and blocked at the 3′-end. In another embodiment, the ssDNA ligation reaction uses a T4 RNA ligase (available from New England BioLabs) to ligate the ssDNA adapter to the 3′-OH end of a bisulfite-converted ssDNA molecule.

In a second step, a second strand DNA is synthesized in an extension reaction. For example, an extension primer, that hybridizes to a primer sequence included in the ssDNA adapter, is used in a primer extension reaction to form a double-stranded bisulfite-converted DNA molecule. Optionally, in some embodiments, the extension reaction uses an enzyme that is able to read through uracil residues in the bisulfite-converted template strand.

Optionally, in a third step, a dsDNA adapter is added to the double-stranded bisulfite-converted DNA molecule. Then, the double-stranded bisulfite-converted DNA can be amplified to add sequencing adapters. For example, PCR amplification using a forward primer that includes a P5 sequence and a reverse primer that includes a P7 sequence is used to add P5 and P7 sequences to the bisulfite-converted DNA. Optionally, during library preparation, unique molecular identifiers (UMI) can be added to the nucleic acid molecules (e.g., DNA molecules) through adapter ligation. The UMIs are short nucleic acid sequences (e.g., 4-10 base pairs) that are added to ends of DNA fragments during adapter ligation. In some embodiments, UMIs are degenerate base pairs that serve as a unique tag that can be used to identify sequence reads originating from a specific DNA fragment. During PCR amplification following adapter ligation, the UMIs are replicated along with the attached DNA fragment, which provides a way to identify sequence reads that came from the same original fragment in downstream analysis.

In an optional step 325, the nucleic acids (e.g., fragments) can be hybridized. Hybridization probes (also referred to herein as “probes”) can be used to target, and pull down, nucleic acid fragments informative for disease states. For a given workflow, the probes can be designed to anneal (or hybridize) to a target (or a complementary) strand of DNA or RNA. The target strand can be the “positive” strand (e.g., the strand transcribed into mRNA, and subsequently translated into a protein) or the complementary “negative” strand. The probes can range in length from 10s, 100s, or 1000s of base pairs. Moreover, the probes can be tiled to cover overlapping portions of a target region.

In an optional step 330, the hybridized nucleic acid fragments are captured and can be enriched, e.g., amplified using PCR. In some embodiments, targeted DNA nucleic acid fragments can be enriched from the library. This is used, for example, where a targeted panel assay is being performed on the samples. For example, the target nucleic acids can be enriched to obtain enriched nucleic acid sequences that can be subsequently sequenced. In general, any known method in the art can be used to isolate, and enrich for, probe-hybridized targeted nucleic acids. For example, as is well known in the art, a biotin moiety can be added to the 5′-end of the probes (i.e., biotinylated) to facilitate isolation of target nucleic acids hybridized to probes using a streptavidin-coated surface (e.g., streptavidin-coated beads).

In step 335, sequence reads are generated from the nucleic acid sample, e.g., enriched nucleic acid sequences. Sequencing data can be acquired from the enriched nucleic acid sequences by known means in the art. For example, the method can include next generation sequencing (NGS) techniques including synthesis technology (Illumina), pyrosequencing (454 Life Sciences), ion semiconductor technology (Ion Torrent sequencing), single-molecule real-time sequencing (Pacific Biosciences), sequencing by ligation (SOLiD sequencing), nanopore sequencing (Oxford Nanopore Technologies), or paired-end sequencing. In some embodiments, massively parallel sequencing is performed using sequencing-by-synthesis with reversible dye terminators. In other embodiments, sequence data can be acquired, or sequences detected, using amplification based detection means, such as, detection by polymerase chain reaction (PCR), digital PCR (dPCR), quantitative PCR (qPCR), real time PCR (RT-PCR), quantitative real time PCR (qRT-PCR), or other well-known means in the art.

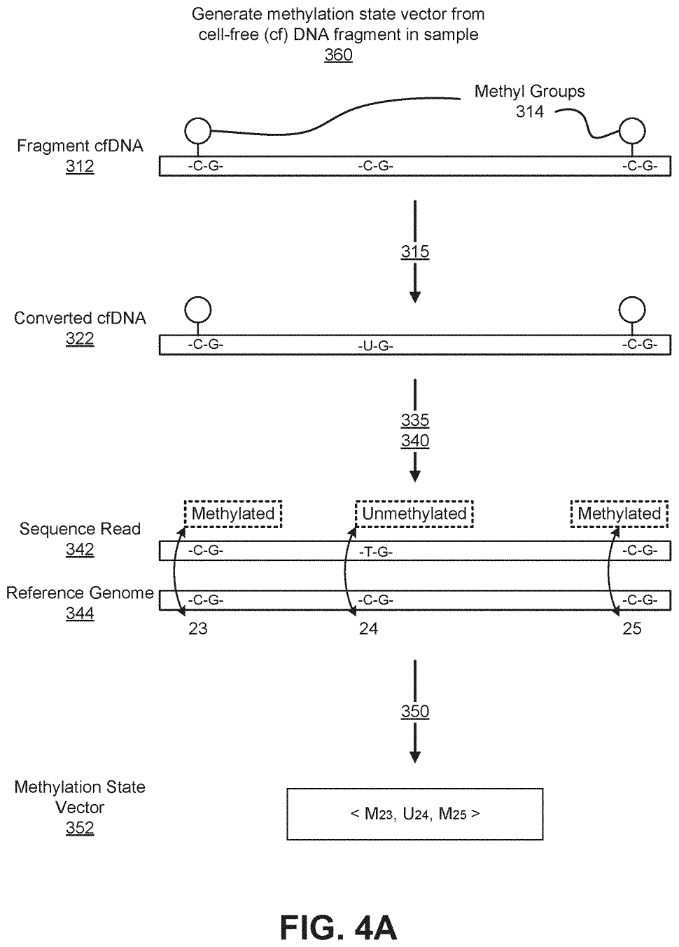

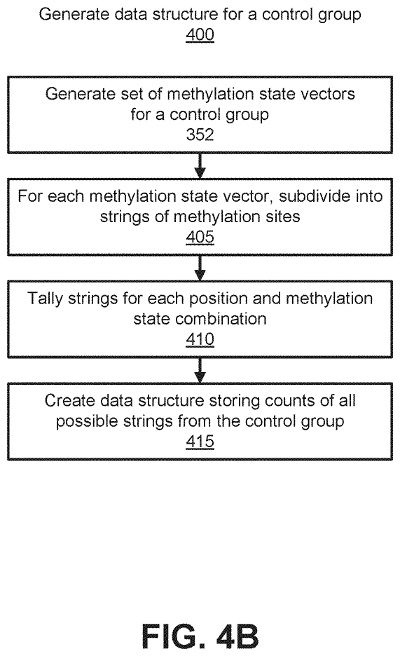

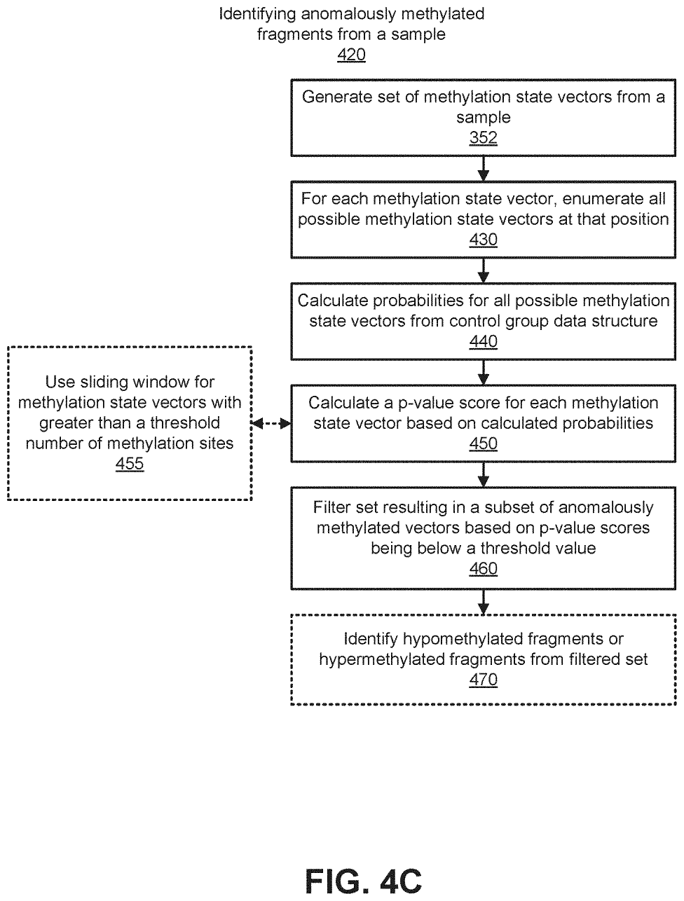

In step 340, the sequence processor 210 can generate methylation information using the sequence reads. A methylation state vector can then be generated using the methylation information determined from the sequence reads. FIG. 4B is an illustration of the process 360, starting from process 300 of FIG. 3 of sequencing a cfDNA molecule, to obtain a methylation state vector 352, according to an embodiment. As an example, the analytics system 200 receives a cfDNA molecule 312 that, in this example, contains three CpG sites. As shown, the first and third CpG sites of the cfDNA molecule 312 are methylated 314. During the treatment step 315, the cfDNA molecule 312 is converted to generate a converted cfDNA molecule 322. During the treatment 315, the second CpG site which was unmethylated has its cytosine converted to uracil. However, the first and third CpG sites were not converted.

After conversion, a sequencing library 330 is prepared and sequenced generating a sequence read 342. The analytics system 200 aligns (not shown) the sequence read 342 to a reference genome 344. The reference genome 344 provides the context as to what position in a human genome the fragment cfDNA originates from. In this simplified example, the analytics system 200 aligns the sequence read 342 such that the three CpG sites correlate to CpG sites 23, 24, and 25 (arbitrary reference identifiers used for convenience of description). The analytics system 200 thus generates information both on methylation status of all CpG sites on the cfDNA molecule 312 and the position in the human genome that the CpG sites map to. As shown, the CpG sites on sequence read 342 which were methylated are read as cytosines. In this example, the cytosines appear in the sequence read 342 only in the first and third CpG site which allows one to infer that the first and third CpG sites in the original cfDNA molecule were methylated. Whereas, the second CpG site is read as a thymine (U is converted to T during the sequencing process), and thus, one can infer that the second CpG site was unmethylated in the original cfDNA molecule. With these two pieces of information, the methylation status and location, the analytics system 200 generates a methylation state vector 352 for the fragment cfDNA 312. In this example, the resulting methylation state vector 352 is <M₂₃, U₂₄, M₂₅>, wherein M corresponds to a methylated CpG site, U corresponds to an unmethylated CpG site, and the subscript number corresponds to a position of each CpG site in the reference genome.

### Identifying Anomalous Fragments

In some embodiments, the analytics system 200 determines anomalous fragments for a sample using the sample's methylation state vectors. For example, for each nucleic acid molecule or fragment in a sample, the analytics system 200 determines whether the nucleic acid molecule or fragment is an anomalously or abnormally methylated molecule or fragment (via analysis of sequence reads derived therefrom), relative to an expected methylation state vector from a healthy sample using the methylation state vector corresponding to the nucleic acid molecule. In some embodiments, the analytics system 200 calculates a p-value score for each methylation state vector describing a probability of observing that methylation state vector or other methylation state vectors even less probable in the healthy control group (as described, for example, in U.S. Pat. Appl. Pub. No. 2019/0287652, which is incorporated herein by reference in its entirety). The process for calculating a p-value score will also be discussed below in Section P-Value Filtering. The analytics system 200 can determine, and optionally filter out, sequence reads of nucleic acid molecules or fragments with a methylation state vector having below a threshold p-value score as anomalous fragments. In another embodiment, the analytics system 200 further labels fragments with at least some number of CpG sites that have over some threshold percentage of methylation or unmethylation as hypermethylated and hypomethylated fragments, respectively. A hypermethylated fragment or a hypomethylated fragment can also be referred to as an unusual fragment with extreme methylation (UFXM). In other embodiments, the analytics system 200 can implement various other probabilistic models for determining anomalous molecules or fragments. Examples of other probabilistic models include a mixture model, a deep probabilistic model, etc. In some embodiments, the analytics system 200 can use any combination of the processes described below for identifying anomalous fragments. With the identified anomalous fragments, the analytics system 200 can filter the set of methylation state vectors for a sample for use in other processes, e.g., for use in training and deploying a cancer classifier.

### P-Value Filtering

In one embodiment, the analytics system 200 calculates a p-value score for each methylation state vector compared to methylation state vectors from fragments in a healthy control group. The p-value score describes a probability of observing a nucleic acid molecule having the methylation status matching that methylation state vector in the healthy control group. In order to determine a DNA fragment to be anomalously methylated, the analytics system 200 uses a healthy control group with a majority of fragments that are normally methylated. When conducting this probabilistic analysis for determining anomalous fragments, the determination holds weight in comparison with the group of control subjects that make up the healthy control group. To ensure robustness in the healthy control group, the analytics system 200 can select some threshold number of healthy individuals to source samples including DNA fragments. FIG. 4B below describes the method of generating a data structure for a healthy control group with which the analytics system 200 can calculate p-value scores. FIG. 4C describes the method of calculating a p-value score with the generated data structure.

FIG. 4B is a flowchart describing a process 400 of generating a data structure for a healthy control group, according to an embodiment. To create a healthy control group data structure, the analytics system 200 receives a plurality of DNA fragments (e.g., cfDNA) from a plurality of healthy individuals. A methylation state vector is identified for each fragment, for example via the process 360.

With each fragment's methylation state vector, the analytics system 200 subdivides 405 the methylation state vector into strings of CpG sites. In one embodiment, the analytics system 200 subdivides 405 the methylation state vector such that the resulting strings are all less than a given length. For example, a methylation state vector of length 11 can be subdivided into strings of length less than or equal to 3 would result in 9 strings of length 3, 10 strings of length 2, and 11 strings of length 1. In another example, a methylation state vector of length 7 being subdivided into strings of length less than or equal to 4 would result in 4 strings of length 4, 5 strings of length 3, 6 strings of length 2, and 7 strings of length 1. If a methylation state vector is shorter than or the same length as the specified string length, then the methylation state vector can be converted into a single string containing all of the CpG sites of the vector.

The analytics system 200 tallies 410 the strings by counting, for each possible CpG site and possibility of methylation states in the vector, the number of strings present in the control group having the specified CpG site as the first CpG site in the string and having that possibility of methylation states. For example, at a given CpG site and considering string lengths of 3, there are 2\hat{ }3 or 8 possible string configurations. At that given CpG site, for each of the 8 possible string configurations, the analytics system 200 tallies 410 how many occurrences of each methylation state vector possibility come up in the control group. Continuing this example, this may involve tallying the following quantities: <Mₓ, Mₓ₊₁, Mₓ₊₂>, <Mₓ, Mₓ₊₁, Uₓ₊₂>, . . . , <Uₓ, Uₓ₊₁, Uₓ₊₂> for each starting CpG site x in the reference genome. The analytics system 200 creates 415 the data structure storing the tallied counts for each starting CpG site and string possibility.

There are several benefits to setting an upper limit on string length. First, depending on the maximum length for a string, the size of the data structure created by the analytics system 200 can dramatically increase in size. For instance, a maximum string length of 4 means that every CpG site has at the very least 2\hat{ }4 numbers to tally for strings of length 4. Increasing the maximum string length to 5 means that every CpG site has an additional 2\hat{ }4 or 16 numbers to tally, doubling the numbers to tally (and computer memory required) compared to the prior string length. Reducing string size helps keep the data structure creation and performance (e.g., use for later accessing as described below), in terms of computational and storage, reasonable. Second, a statistical consideration to limiting the maximum string length is to avoid overfitting downstream models that use the string counts. If long strings of CpG sites do not, biologically, have a strong effect on the outcome (e.g., predictions of anomalousness that predictive of the presence of cancer), calculating probabilities based on large strings of CpG sites can be problematic as it requires a significant amount of data that may not be available, and thus would be too sparse for a model to perform appropriately. For example, calculating a probability of anomalousness/cancer conditioned on the prior 100 CpG sites would require counts of strings in the data structure of length 100, ideally some matching exactly the prior 100 methylation states. If only sparse counts of strings of length 100 are available, there will be insufficient data to determine whether a given string of length of 100 in a test sample is anomalous or not.

FIG. 4C is a flowchart describing a process 420 for identifying anomalously methylated fragments from an individual, according to an embodiment. In process 420, the analytics system 200 generates methylation state vectors 352 from cfDNA fragments of the subject. The analytics system 200 handles each methylation state vector as follows.

For a given methylation state vector, the analytics system 200 enumerates 430 all possibilities of methylation state vectors having the same starting CpG site and same length (i.e., set of CpG sites) in the methylation state vector. As each methylation state is generally either methylated or unmethylated there are effectively two possible states at each CpG site, and thus the count of distinct possibilities of methylation state vectors depends on a power of 2, such that a methylation state vector of length n would be associated with 2ⁿ possibilities of methylation state vectors. With methylation state vectors inclusive of indeterminate states for one or more CpG sites, the analytics system 200 can enumerate 430 possibilities of methylation state vectors considering only CpG sites that have observed states.

The analytics system 200 calculates 440 the probability of observing each possibility of methylation state vector for the identified starting CpG site and methylation state vector length by accessing the healthy control group data structure. In one embodiment, calculating the probability of observing a given possibility uses a Markov chain probability to model the joint probability calculation. In other embodiments, calculation methods other than Markov chain probabilities are used to determine the probability of observing each possibility of methylation state vector.

The analytics system 200 calculates 450 a p-value score for the methylation state vector using the calculated probabilities for each possibility. In one embodiment, this includes identifying the calculated probability corresponding to the possibility that matches the methylation state vector in question. Specifically, this is the possibility of having the same set of CpG sites, or similarly the same starting CpG site and length as the methylation state vector. The analytics system 200 sums the calculated probabilities of any possibilities having probabilities less than or equal to the identified probability to generate the p-value score.

This p-value represents the probability of observing the methylation state vector of the fragment or other methylation state vectors even less probable in the healthy control group. A low p-value score, thereby, generally corresponds to a methylation state vector which is rare in a healthy individual, and which causes the fragment to be labeled anomalously methylated, relative to the healthy control group. A high p-value score generally relates to a methylation state vector is expected to be present, in a relative sense, in a healthy individual. If the healthy control group is a non-cancerous group, for example, a low p-value indicates that the fragment is anomalously methylated relative to the non-cancer group, and therefore possibly indicative of the presence of cancer in the test subject.

As above, the analytics system 200 calculates p-value scores for each of a plurality of methylation state vectors, each representing a cfDNA fragment in the test sample. To identify which of the fragments are anomalously methylated, the analytics system 200 can filter 460 the set of methylation state vectors based on their p-value scores. In one embodiment, filtering is performed by comparing the p-values scores against a threshold and keeping only those fragments below the threshold. This threshold p-value score could be on the order of 0.1, 0.01, 0.001, 0.0001, or similar.

According to example results from the process, the analytics system 200 yields a median (range) of 2,800 (1,500-12,000) fragments with anomalous methylation patterns for participants without cancer in training, and a median (range) of 3,000 (1,200-220,000) fragments with anomalous methylation patterns for participants with cancer in training. These filtered sets of fragments with anomalous methylation patterns may be used for the downstream analyses as described below.

In some embodiments, the analytics system 200 uses 455 a sliding window to determine possibilities of methylation state vectors and calculate p-values. Rather than enumerating possibilities and calculating p-values for entire methylation state vectors, the analytics system 200 enumerates possibilities and calculates p-values for only a window of sequential CpG sites, where the window is shorter in length (of CpG sites) than at least some fragments (otherwise, the window would serve no purpose). The window length may be static, user determined, dynamic, or otherwise selected.

In calculating p-values for a methylation state vector larger than the window, the window identifies the sequential set of CpG sites from the vector within the window starting from the first CpG site in the vector. The analytic system 200 calculates a p-value score for the window including the first CpG site. The analytics system 200 then “slides” the window to the second CpG site in the vector, and calculates another p-value score for the second window. Thus, for a window size l and methylation vector length m, each methylation state vector will generate m−l+1 p-value scores. After completing the p-value calculations for each portion of the vector, the lowest p-value score from all sliding windows is taken as the overall p-value score for the methylation state vector. In another embodiment, the analytics system 200 aggregates the p-value scores for the methylation state vectors to generate an overall p-value score.

Using the sliding window helps to reduce the number of enumerated possibilities of methylation state vectors and their corresponding probability calculations that would otherwise need to be performed. To give a realistic example, it is possible for fragments to have upwards of 54 CpG sites. Instead of computing probabilities for 2\hat{ }54 (˜1.8×10\hat{ }16) possibilities to generate a single p-score, the analytics system 200 can instead use a window of size 5 (for example) which results in 50 p-value calculations for each of the 50 windows of the methylation state vector for that fragment. Each of the 50 calculations enumerates 2\hat{ }5 (32) possibilities of methylation state vectors, which total results in 50×2\hat{ }5 (1.6×10\hat{ }3) probability calculations. This results in a vast reduction of calculations to be performed, with no meaningful hit to the accurate identification of anomalous fragments.

In embodiments with indeterminate states, the analytics system 200 can calculate a p-value score summing out CpG sites with indeterminate states in a fragment's methylation state vector. The analytics system 200 identifies all possibilities that have consensus with the all methylation states of the methylation state vector excluding the indeterminate states. The analytics system 200 can assign the probability to the methylation state vector as a sum of the probabilities of the identified possibilities. As an example, the analytics system 200 calculates a probability of a methylation state vector of <M₁, I₂, U₃> as a sum of the probabilities for the possibilities of methylation state vectors of <M₁, M₂, U₃> and <M₁, U₂, U₃> since methylation states for CpG sites 1 and 3 are observed and in consensus with the fragment's methylation states at CpG sites 1 and 3. This method of summing out CpG sites with indeterminate states uses calculations of probabilities of possibilities up to 2\hat{ }i, wherein i denotes the number of indeterminate states in the methylation state vector. In additional embodiments, a dynamic programming algorithm can be implemented to calculate the probability of a methylation state vector with one or more indeterminate states. Advantageously, the dynamic programming algorithm operates in linear computational time.

In some embodiments, the computational burden of calculating probabilities and/or p-value scores can be further reduced by caching at least some calculations. For example, the analytic system 200 can cache in transitory or persistent memory calculations of probabilities for possibilities of methylation state vectors (or windows thereof). If other fragments have the same CpG sites, caching the possibility probabilities allows for efficient calculation of p-score values without needing to re-calculate the underlying possibility probabilities. Equivalently, the analytics system 200 can calculate p-value scores for each of the possibilities of methylation state vectors associated with a set of CpG sites from vector (or window thereof). The analytics system 200 can cache the p-value scores for use in determining the p-value scores of other fragments including the same CpG sites. Generally, the p-value scores of possibilities of methylation state vectors having the same CpG sites can be used to determine the p-value score of a different one of the possibilities from the same set of CpG sites.

### Hypermethylated Fragments and Hypomethylated Fragments

In some embodiments, the analytics system 200 determines anomalous fragments as fragments with over a threshold number of CpG sites and either with over a threshold percentage of the CpG sites methylated or with over a threshold percentage of CpG sites unmethylated; the analytics system 200 identifies such fragments as hypermethylated fragments or hypomethylated fragments. Example thresholds for length of fragments (or CpG sites) include more than 3, 4, 5, 6, 7, 8, 9, 10, etc. Example percentage thresholds of methylation or unmethylation include more than 80%, 85%, 90%, or 95%, or any other percentage within the range of 50%-100%.

### Exemplary Sequencer and Analytics System

FIGS. 2A&B are a flowchart of systems and devices for sequencing nucleic acid samples according to some embodiments. This illustrative flowchart includes devices such as a sequencer 270 and a processing system (e.g., analytics system 200). The sequencer 270 and the analytics system 200 can work in tandem to perform one or more steps in the processes described herein.

In various embodiments, the sequencer 270 receives an enriched nucleic acid sample 260. As shown in FIG. 2A, the sequencer 270 can include a graphical user interface 275 that enables user interactions with particular tasks (e.g., initiate sequencing or terminate sequencing) as well as one more loading stations 280 for loading a sequencing cartridge including the enriched fragment samples and/or for loading necessary buffers for performing the sequencing assays. Therefore, once a user of the sequencer 270 has provided the necessary reagents and sequencing cartridge to the loading station 280 of the sequencer 270, the user can initiate sequencing by interacting with the graphical user interface 275 of the sequencer 270. Once initiated, the sequencer 270 performs the sequencing and outputs the sequence reads of the enriched fragments from the nucleic acid sample 260.

In some embodiments, the sequencer 270 is communicatively coupled with the analytics system 200. The analytics system 200 includes some number of computing devices used for processing the sequence reads for various applications such as assessing methylation status at one or more CpG sites, variant calling or quality control. The sequencer 270 can provide the sequence reads in a BAM file format to the analytics system 200. The analytics system 200 can be communicatively coupled to the sequencer 270 through a wireless, wired, or a combination of wireless and wired communication technologies. Generally, the analytics system 200 is configured with a processor and non-transitory computer-readable storage medium storing computer instructions that, when executed by the processor, cause the processor to process the sequence reads or to perform one or more steps of any of the methods or processes disclosed herein.

In some embodiments, the sequence reads can be aligned to a reference genome using known methods in the art to determine alignment position information. Alignment position can generally describe a beginning position and an end position of a region in the reference genome that corresponds to a beginning nucleotide base and an end nucleotide base of a given sequence read. Corresponding to methylation sequencing, the alignment position information can be generalized to indicate a first CpG site and a last CpG site included in the sequence read according to the alignment to the reference genome. The alignment position information can further indicate methylation statuses and locations of all CpG sites in a given sequence read. A region in the reference genome can be associated with a gene or a segment of a gene; as such, the analytics system 200 can label a sequence read with one or more genes that align to the sequence read. In some embodiments, fragment length (or size) is determined from the beginning and end positions.

In various embodiments, for example when a paired-end sequencing process is used, a sequence read is comprised of a read pair denoted as R_1 and R_2. For example, the first read R_1 may be sequenced from a first end of a double-stranded DNA (dsDNA) molecule whereas the second read R_2 may be sequenced from the second end of the double-stranded DNA (dsDNA). Therefore, nucleotide base pairs of the first read R_1 and second read R_2 can be aligned consistently (e.g., in opposite orientations) with nucleotide bases of the reference genome. Alignment position information derived from the read pair R_1 and R_2 can include a beginning position in the reference genome that corresponds to an end of a first read (e.g., R_1) and an end position in the reference genome that corresponds to an end of a second read (e.g., R_2). In other words, the beginning position and end position in the reference genome represent the likely location within the reference genome to which the nucleic acid fragment corresponds. In some embodiments, the read pair R_1 and R_2 can be assembled into a fragment, and the fragment used for subsequent analysis and/or classification. An output file having SAM (sequence alignment map) format or BAM (binary) format can be generated and output for further analysis.

Referring back to FIG. 2B, FIG. 2B is a block diagram of the analytics system 200 for processing DNA samples according to some embodiments. The analytics system 200 implements one or more computing devices for use in analyzing DNA samples. The analytics system 200 includes a sequence processor 210, sequence database 215, model database 225, one or more probabilistic models 230 and/or one or more classifiers 240, and parameter database 235. In some embodiments, the analytics system 200 performs one or more steps in the methods or processes disclosed herein.

The sequence processor 210 generates methylation state vectors for fragments from a sample. At each CpG site on a fragment, the sequence processor 210 generates a methylation state vector for each fragment specifying a location of the fragment in the reference genome, a number of CpG sites in the fragment, and the methylation state of each CpG site in the fragment whether methylated, unmethylated, or indeterminate via the process 360 of FIG. 4B. The sequence processor 210 can store methylation state vectors for fragments in the sequence database 215. Data in the sequence database 215 can be organized such that the methylation state vectors from a sample are associated with one another.

Further, multiple different models 230 can be stored in the model database 225 or retrieved for use with test samples. In one example, a model is a trained cancer classifier 240 for determining a cancer prediction for a test sample using a feature vector derived from anomalous fragments. The training and use of the cancer classifier is discussed elsewhere herein. The analytics system 200 can train the one or more models 230 and/or one or more classifiers 240 and store various trained parameters in the parameter database 235. The analytics system 200 stores the models 230 and/or classifiers along with functions in the model database 225.

During inference, the machine learning engine 220 uses the one or more models 230 and/or classifiers 240 to return outputs. The machine learning engine accesses the models 230 and/or classifiers 240 in the model database 225 along with trained parameters from the parameter database 235. According to each model, the machine learning engine 220 receives an appropriate input for the model and calculates an output based on the received input, the parameters, and a function of each model relating the input and the output. In some use cases, the machine learning engine 220 further calculates metrics correlating to a confidence in the calculated outputs from the model. In other use cases, the machine learning engine 220 calculates other intermediary values for use in the model.

### Blocks of Reference Genome

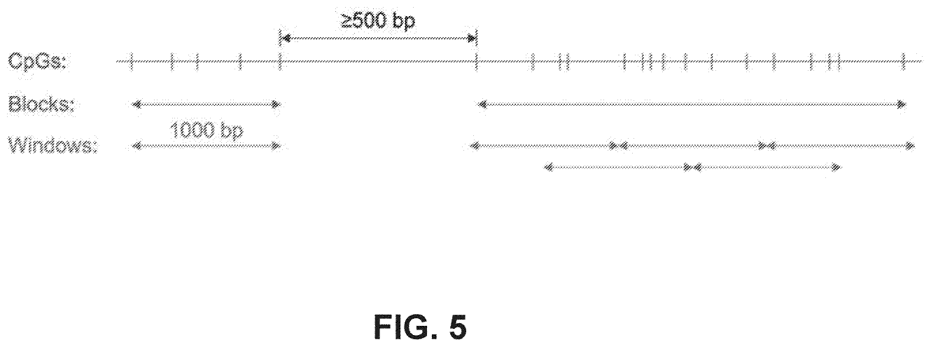

Turning now to FIG. 5, FIG. 5 is an illustration of blocks of a reference genome, according to some embodiments. The sequence processor 210 can partition a reference genome (or a subset of the reference genome) in one or more stages, e.g., for use cases involving a targeted methylation assay. For instance, the sequence processor 210 separates the reference genome into blocks of CpG sites. Each block is defined when there is a separation between two adjacent CpG sites that exceeds a threshold, e.g., greater than 200 base pairs (bp), 300 bp, 400 bp, 500 bp, 600 bp, 700 bp, 800 bp, 900 bp, or 1,000 bp, among other values. Thus, blocks can vary in size of base pairs. For each block, the sequence processor 210 can subdivide the block into windows of a certain length, e.g., 500 bp, 600 bp, 700 bp, 800 bp, 900 bp, 1,000 bp, 1,100 bp, 1,200 bp, 1,300 bp, 1,400 bp, or 1,500 bp, among other values. In other embodiments, the windows can be from 200 bp to 10 kilobase pairs (kbp), from 500 bp to 2 kbp, or about 1 kbp in length. Windows (e.g., that are adjacent) can overlap by a number of base pairs or a percentage of the length, e.g., 10%, 20%, 30%, 40%, 50%, or 60%, among other values. Windows can be separated between two adjacent CpG sites that exceeds a threshold, e.g., greater than 200 base pairs (bp), 300 bp, 400 bp, 500 bp, 600 bp, 700 bp, 800 bp, 900 bp, or 1,000 bp, among other values.

The sequence processor 210 can analyze sequence reads derived from DNA fragments using a windowing process. In particular, the sequence processor 210 scans through the blocks window-by-window and reads fragments within each window. The fragments can originate from tissue and/or high-signal cfDNA. High-signal cfDNA samples can be determined by a binary classification model, by cancer stage, or by another metric. By partitioning the reference genome (e.g., using blocks and windows), the sequence processor 210 can facilitate computational parallelization. Moreover, the sequence processor 210 can reduce computational resources to process a reference genome by targeting the sections of base pairs that include CpG sites, while skipping other sections that do not include CpG sites.

### Model Based Feature Engineering and Classification

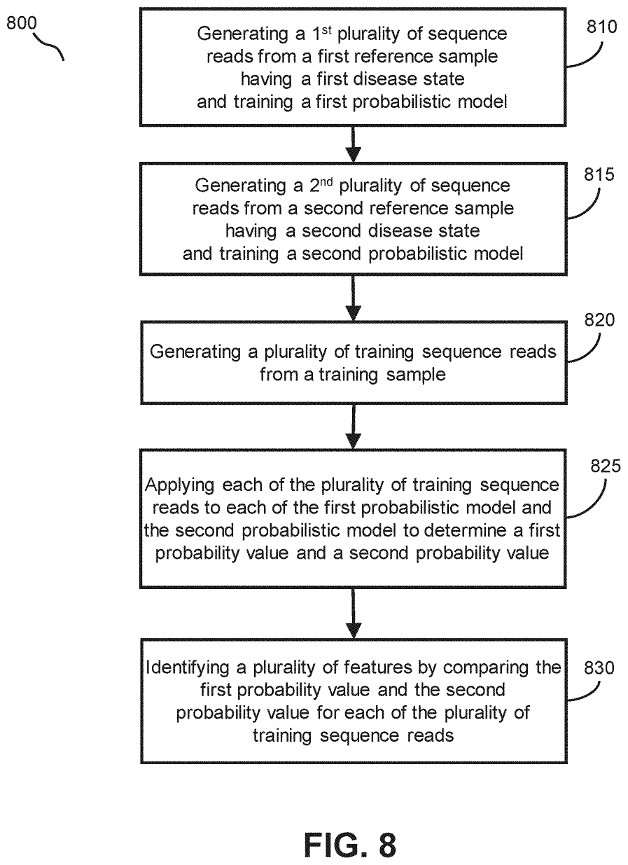

Turning now to FIG. 8, in accordance with some embodiments, the present disclosure is directed to model-based feature engineering for deriving features useful for classification of a disease state. As described elsewhere herein, the disease state can be the presence or absence of a disease, a type of disease, and/or a disease tissue or origin. For example, as described herein, the disease state can be the presence or absence of cancer, a type of cancer, and/or a cancer tissue of origin. The type of cancer and/or cancer tissue of origin can be selected from the group including breast cancer, uterine cancer, cervical cancer, ovarian cancer, bladder cancer, urothelial cancer of renal pelvis, renal cancer other than urothelial, prostate cancer, anorectal cancer, colorectal cancer, esophageal cancer, gastric cancer, hepatobiliary cancer arising from hepatocytes, hepatobiliary cancer arising from cells other than hepatocytes, pancreatic cancer, squamous cell cancer of the upper gastrointestinal tract, upper gastrointestinal cancer other than squamous, head and neck cancer, lung cancer, such as lung adenocarcinoma, small cell lung cancer, squamous cell lung cancer and cancer other than adenocarcinoma or small cell lung cancer, neuroendocrine cancer, melanoma, thyroid cancer, sarcoma, multiple myeloma, lymphoma, and leukemia, among other types of cancer.

In step 810, a first plurality of sequence reads are generated, as described elsewhere herein, from a first reference sample having a first disease state, and a second plurality of sequence reads are generated from a second reference sample having a second disease state. The first plurality of sequence reads and/or the second plurality of sequence reads can be more than 10,000, more than 50,000, more than 100,000, more than 200,000, more than 500,000, more than 1,000,000, more than 2,000,000, more than 5,000,000, or more than 10,000,000 sequence reads. As used herein a “reference sample” is a sample obtained from a subject with a known disease state. In some embodiments, one or more reference samples, having one or more known disease states, can be used to train one or more probabilistic models, that in turn can be used to derive features for classifying a disease state of an unknown test sample. The sample can be a genomic DNA (gDNA) sample or a cell free DNA (cfDNA) sample. The reference sample can be a blood, plasma, serum, urine, fecal, and saliva samples. Alternatively, the reference sample can be whole blood, a blood fraction, a tissue biopsy, pleural fluid, pericardial fluid, cerebral spinal fluid, and peritoneal fluid. In some embodiments, the first reference sample is obtained from a subject known to have cancer and the second reference sample is obtained from a healthy subject or a non-cancer subject. In some embodiments, the first reference sample is obtained from a subject known to have a first type of cancer (e.g., lung cancer) and the second reference sample is obtained from a subject known to have a second type of cancer (e.g., breast cancer). In still other embodiments, the first reference sample is obtained from a subject known to have a first disease tissue of origin (e.g., lung disease) and a second reference sample is obtained from a second disease state tissue of origin (e.g., a liver disease).

In step 815, the machine learning engine 220 trains a first probabilistic model 230 and a second probabilistic model 230, from the first plurality of sequence reads and the second plurality of sequence reads (generated in step 110), respectively, each probabilistic model associated with a different disease state of one or more possible disease states. As previously described, the disease state can be the presence or absence of cancer, a type of cancer, and/or a cancer tissue of origin. In various embodiments, training data is split into K subsets (folds) for K-fold cross-validation. Folds can be balanced for: cancer/non-cancer status, tissue of origin, cancer stage, age (e.g., grouped in 10-year buckets), gender, ethnicity, and smoking status, among other factors. Data from K−1 of the folds can be used as training data for the probabilistic models, and the held-out fold can be used as testing data.

The machine learning engine 220 trains the first and second probabilistic models 230, for the first and second disease states, respectively, by fitting each of the probabilistic models 230 to the first plurality and second plurality of sequence reads, respectively. For example, in one embodiment, the first probabilistic model is fitted using a first plurality of sequence reads derived from one or more samples from subjects known to have cancer and the second probabilistic model is fitted using the second plurality of sequence reads derived from one or more samples from healthy subjects or non-cancer subjects. In other embodiments, the first probabilistic model can be trained for a first type of cancer or a first tissue of origin and the second probabilistic model can be trained for a second type of cancer or a second tissue of origin. As one of skill in the art would appreciate, any number of disease state probabilistic models can be trained utilizing sequence reads derived from one or more samples taken from subjects with any one of a number of possible disease states. For example, in some embodiments, additional cancer-specific probabilistic models (i.e., for additional types of cancer and or tissues of origin models) can be trained for a third, fourth, fifth, sixth, seventh, eighth, ninth, tenth, etc. (e.g., up to twenty, thirty, or more) specific type of cancer and used to determine probabilities that sequence reads from a training set, or an unknown cancer type, are more likely derived from one cancer type (or cancer tissue of origin) than another cancer type (or cancer tissue of origin), as described elsewhere herein.

As used herein a “probabilistic model” is any mathematical model capable of assigning a probability to a sequence read based on methylation status at one or more sites on the read. During training, the machine learning engine 220 fits sequence reads derived from one or more samples from subjects having a known disease and can be used to determine sequence reads probabilities indicative of a disease state utilizing methylation information or methylation state vectors (e.g., previously described with respect to FIGS. 3-4). In particular, in one embodiment, the machine learning engine 220 determines observed rates of methylation for each CpG site within a sequence read. The rate of methylation represents a fraction or percentage of base pairs that are methylated within a CpG site. The trained probabilistic model 230 can be parameterized by products of the rates of methylation. In general, any known probabilistic model for assigning probabilities to sequence reads from a sample can be used. For example, the probabilistic model can be a binomial model, in which every site (e.g., CpG site) on a nucleic acid fragment is assigned a probability of methylation, or an independent sites model, in which each CpG's methylation is specified by a distinct methylation probability with methylation at one site assumed to be independent of methylation at one or more other sites on the nucleic acid fragment.

In some embodiments, the probabilistic model 230 is a Markov model, in which the probability of methylation at each CpG site is dependent on the methylation state at some number of preceding CpG sites in the sequence read, or nucleic acid molecule from which the sequence read is derived. See, e.g., U.S. Pat. Appl. Pub. No. 2019/0287652, filed Mar. 13, 2019 and entitled “Anomalous Fragment Detection and Classification,” which is incorporated herein by reference in its entirety.

In some embodiments, the probabilistic model 230 is a “mixture model” fitted using a mixture of components from underlying models. For example, in some embodiments, the mixture components can be determined using multiple independent sites models, where methylation (e.g., rates of methylation) at each CpG site is assumed to be independent of methylation at other CpG sites. Utilizing an independent sites model, the probability assigned to a sequence read, or the nucleic acid molecule from which it derives, is the product of the methylation probability at each CpG site where the sequence read is methylated and one minus the methylation probability at each CpG site where the sequence read is unmethylated. In accordance with this embodiment, the machine learning engine 220 determines rates of methylation of each of the mixture components. The mixture model is parameterized by a sum of the mixture components each associated with a product of the rates of methylation. A probabilistic model Pr of n mixture components can be represented as:

$$Pr ( fragment \vert \{ {β}_{ki} , {f}_{k} \} ) = {\sum }_{k = 1}^{n} {f}_{k} {\prod }_{i}^{ } {{β}_{ki}^{{m}_{i}} ( 1 - {β}_{ki} )}^{1 - {m}_{i}}$$

For an input fragment, mᵢ ∈{0, 1} represents the fragment's observed methylation status at position i of a reference genome, with 0 indicating unmethylation and 1 indicating methylation. A fractional assignment to each mixture component k is fₖ, where fₖ≥0 and Σₖ₌₁ⁿ fₖ=1. The probability of methylation at position i in a CpG site of mixture component k is βₖᵢ. Thus, the probability of unmethylation is 1−βₖᵢ. The number of mixture components n can be 1, 2, 3, 4, 5, 6, 7, 8, 9, 10, etc.

In some embodiments, the machine learning engine 220 fits the probabilistic model 230 using maximum-likelihood estimation to identify a set of parameters {βₖᵢ, fₖ} that maximizes the log-likelihood of all fragments deriving from a disease state, subject to a regularization penalty applied to each methylation probability with regularization strength r. The maximized quantity for N total fragments can be represented as:

$${\sum }_{j}^{N} ln ( Pr ( {fragment}_{j} \vert \{ {β}_{ki} , {f}_{k} \} ) ) + r \cdot ln ( {β}_{ki} ( 1 - {β}_{ki} ) )$$

As one of skill in the art would appreciate, other means can be used to fit the probabilistic models or to identify parameters that maximize the log-likelihood of all sequence reads derived from the reference samples. For example, in one embodiment, Bayesian fitting (using e.g., Markov chain Monte Carlo), in which each parameter is not assigned a single value but instead is associated to a distribution, is used. In other embodiments, gradient-based optimization, in which the gradient of the likelihood (or log-likelihood) with respect to the parameter values is used to step through parameter space towards an optimum, is used. In other embodiments, expectation-maximization, in which a set of latent parameters (such as identities of the mixture component from which each fragment is derived) are set to their expected values under the previous model parameters, and then the model's parameters are assigned to maximize the likelihood conditional on the assumed values of those latent variables. The two-step process is then repeated until convergence.

At step 820, a plurality of training sequence reads are generated from a training sample. The plurality of training sequence reads can be more than 10,000, more than 50,000, more than 100,000, more than 200,000, more than 500,000, more than 1,000,000, more than 2,000,000, more than 5,000,000, or more than 10,000,000 sequence reads. As used herein, a “training sample” is a sample obtained from a known disease state that can be used to generate sequence reads, which are then applied to the first and/or second probability models to generate features that can be utilized for disease state classification. In step 825, the analytics system 200 applies the first and second probabilistic models 230 to determine a first probability value and a second probability value for each sequence read of the plurality of training sequence reads. The first and second probability values are determined based on a probability that the sequence read originated from a sample associated with the first disease state, and the second disease state, respectively. The analytics system 200 can repeat step 130 for any additional probabilistic models 230 (e.g., trained from sequence reads from a third, fourth, fifth, etc. reference sample) (not shown).

At step 830 one or more features are identified by comparing the first probability value and the second probability value for each of the plurality of training sequence reads. In general, a wide array of methods can be utilized to compare the first and second probability values and identify features. For example, in one embodiment, the one or more features comprise a count of outlier sequence reads of the plurality of training sequence reads where the first probability value is greater than the second probability value. The count can be a binary count, a total count of outlier sequence reads, or a total count of anonymously methylated sequence reads. In another embodiment, the one or more features comprises a count of sequence reads or fragments including a particular methylation pattern. For example, the one or more features can be a count of sequence reads or fragments that are fully methylated at each CpG site, a count of sequence reads or fragments that are partially methylated (e.g., at least 20%, 30%, 40%, 50%, 60%, 70%, 80%, 90%, or 95% methylated). In another embodiment, the one or more features are identified using an output of a discriminative classifier trained within a single genomic region (e.g., the discriminative classifier can be a multilayer perceptron or a convolutional neural net model). In another embodiment, comparing the first probability value and the second probability value comprises determining a ratio of the first probability value and the second probability value, and the one or more features comprise sequence read counts of sequence reads that exceed a ratio threshold value.

In another embodiment, the first probability value or the second probability value is a log-likelihood value. For example, the analytics system 200 can calculate a log-likelihood ratio R with the fitted probabilistic models associated with the first and second disease states, respectively. Specifically, the log-likelihood ratio can be calculated using the probabilities Pr of observing a methylation pattern on the fragment for samples associated with the first disease state and second disease state:

$${R}_{disease state} ( fragment ) ≡ ln ( \frac{Pr ( fragment \vert first disease state )}{Pr ( fragment \vert second disease state )} )$$

The analytics system 200 can identify features using multiple tiers of threshold values. For example, the tiers include threshold values of 1, 2, 3, 4, 5, 6, 7, 8, and 9. In some embodiments, a smoothing function can be applied. For example, responsive to determining that R is (e.g., significantly) less than a tier value, the analytics system 200 assigns a feature value of ˜0; responsive to determining that R equals a tier value, the processing system 200 assigns a feature value of 0.5; responsive to determining that R is (e.g., significantly) greater than a tier value, the processing system 200 assigns a feature value of ˜1. Each tier indicates a varying threshold that a fragment (from which the sequence reads were generated) more likely originated from a sample associated with a disease state than from a healthy sample. The analytics system 200 can use the threshold value to determine counts of outlier fragments, which can be used as features.

By filtering with a threshold value, the analytics system 200 can consider certain fragments as outliers because the fragments are unlikely to be present in healthy samples. Accordingly, outlier fragments can be considered to be more likely associated with (e.g., originating from) a disease state or a cancer sample. The number of features can vary between different tiers, e.g., one tier can have a different number of features than another tier based on the corresponding threshold values. In other embodiments, the analytics system 200 uses a different number of tiers or other threshold values. Other means for identifying features, or ranking the identified features based on measures of the features in distinguishing between different disease states (e.g., using mutual information to determine the measure of information content of a feature in distinguishing between two disease states) are described elsewhere herein.

In other embodiments, the analytics system 200 can identify a plurality of features using a different type of ratio or equation. The machine learning engine 220 can determine a fragment to be indicative of a disease state (e.g., cancer) based on whether at least one of the log-likelihood ratios considered against the various disease states is above a threshold value.

Subsequently, as described in further detail elsewhere herein, the plurality of features can be used to train a disease state classifier. For example, in some embodiments, the plurality of features can be used to train a classifier for classification of the presence or absence of cancer, a type of cancer, and/or a cancer tissue of origin.

### Disease State Tissue of Origin Classification

Turning back to FIG. 1, in accordance with another embodiment, as illustrated in FIG. 1 step 120, the machine learning engine 220 trains probabilistic models 230 each associated with a different disease state of a set of multiple disease states. For clarity, FIG. 1 describes model-based featurization and training of a classifier for classification of a disease state tissue of origin. However, as previously described, in various embodiments, the disease state can be the presence or absence of cancer, a type of cancer, and/or a cancer tissue of origin. Additionally, the disease state can be associated with another type of disease (not necessarily associated with cancer) or a healthy state (no presence of cancer or disease).

The machine learning engine 220 trains probabilistic models 230 using one or more sets of sequence reads, wherein each of the one or more sets of sequence reads are generated (in accordance with step 110) from a different disease state of the set of multiple disease states. The disease states can include any number of types of cancer or cancer tissues of origin selected from the group including breast cancer, uterine cancer, cervical cancer, ovarian cancer, bladder cancer, urothelial cancer of renal pelvis, renal cancer other than urothelial, prostate cancer, anorectal cancer, colorectal cancer, esophageal cancer, gastric cancer, hepatobiliary cancer arising from hepatocytes, hepatobiliary cancer arising from cells other than hepatocytes, pancreatic cancer, squamous cell cancer of the upper gastrointestinal tract, upper gastrointestinal cancer other than squamous, head and neck cancer, lung cancer, such as lung adenocarcinoma, small cell lung cancer, squamous cell lung cancer and cancer other than adenocarcinoma or small cell lung cancer, neuroendocrine cancer, melanoma, thyroid cancer, sarcoma, multiple myeloma, lymphoma, and leukemia, among other types of cancer.

The machine learning engine 220 trains a probabilistic model 230, for each of the plurality of disease states, by fitting the probabilistic model 230 to the sequence reads deriving from each sample corresponding to each of the disease states. For example, in some embodiments, probabilistic models can be trained for specific types of cancer. In accordance with this embodiment, cancer-specific probabilistic models can be trained for a first, second, third, etc. specific type of cancer and used to assess a cancer type (e.g., of an unknown test sample). For example, a lung cancer-specific probabilistic model is fitted using a set of sequence reads deriving from one or more samples associated with lung cancer. As another example, a breast cancer-specific probabilistic model is fitted using a set of sequence reads deriving from one or more samples associated with breast cancer. In some embodiments, tissue specific probability models can be trained for a first, second, third, etc. tissue type and used to assess a disease state tissue of origin. For example, a first tissue of origin probabilistic model can be fitted using a set of sequence reads derived from a first tissue type (e.g., from a lung tissue sample, such as a lung biopsy) and a second tissue of origin probabilistic model can be fitted using a set of sequence reads derived from a second tissue type (e.g., from a liver tissue sample, such as a liver biopsy). Alternatively, in some embodiments, a cancer probabilistic model is fitted using a set of sequence reads derived from one or more samples from subjects known to have cancer and a non-cancer specific probabilistic model is fitted using a set of sequence reads derived from one or more samples from healthy subjects or non-cancer subjects. As one of skill in the art would appreciate, any number of disease state probabilistic models can be trained utilizing sequence reads derived from one or more samples taken from subjects with any one of a number of possible disease states. For example, in some embodiments, a plurality of sequence reads can be generated from a 3, 4, 5, 6, 7, 8, 9, 10, or more reference sample, each obtained from one or more subjects having a different disease state (e.g., different types of cancer), and used to train 3, 4, 5, 6, 7, 8, 9, 10, or more probabilistic models.

During training, the machine learning engine 220 can be trained on sequence reads indicative of a disease state utilizing methylation information or methylation state vectors (e.g., previously described with respect to FIGS. 3-4). In particular, the machine learning engine 220 determines observed rates of methylation for each CpG site within a sequence read. The rate of methylation represents a fraction or percentage of base pairs that are methylated within a CpG site. The trained probabilistic model 230 can be parameterized by products of the rates of methylation. As previously described, any known probabilistic model for assigning probabilities to sequence reads from a sample can be used. For example, the probabilistic model can be a binomial model, in which every site (e.g., CpG site) on a nucleic acid fragment is assigned a probability of methylation, or an independent sites model, in which each CpG's methylation is specified by a distinct methylation probability with methylation at one site assumed to be independent of methylation at one or more other sites on the nucleic acid fragment.

In some embodiments, as discussed previously, a Markov model, in which the probability of methylation at each CpG site is dependent on the methylation state at some number of preceding CpG sites in the sequence read, or nucleic acid molecule from which the sequence read is derived. See, e.g., U.S. Pat. Appl. Pub. No. 2019/0287652, entitled “Anomalous Fragment Detection and Classification,” and filed Mar. 13, 2019, incorporated herein by reference in its entirety.

In some embodiments, the probabilistic model 230 is a “mixture model” fitted using a mixture of components from underlying models, such as the probabilistic model Pr described above. Further, in some embodiments, the machine learning engine 220 fits the probabilistic model 230 using maximum-likelihood estimation, as described above.

In step 130, the analytics system 200 applies a probabilistic model 230 to calculate values for each sequence read of a second set of sequence reads, e.g., different than the first set of sequence reads generated in step 110. The values are calculated based at least on a probability that the sequence read (and corresponding fragment) originated from a sample associated with the disease state of the probabilistic model 230. The analytics system 200 can repeat step 130 for each of the different probabilistic models 230. In some embodiments, the analytics system 200 calculates the value using a log-likelihood ratio R with the fitted probabilistic models associated with certain disease states, such as the R_disease state as described above.

In other embodiments, the analytics system 200 can calculate the value using a different type of ratio or equation. The machine learning engine 220 can determine a fragment to be indicative of a disease state (e.g., cancer) based on whether at least one of the log-likelihood ratios considered against the various disease states is above a threshold value.

### Feature Selection

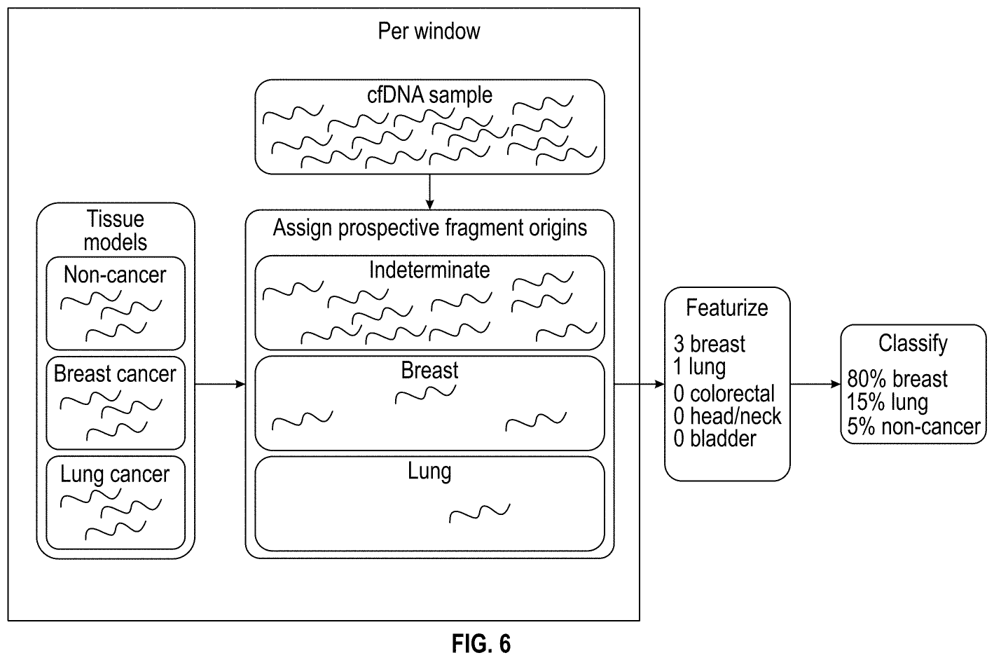

FIG. 6 is an illustration of a process of determining features to train a classifier, according to an embodiment. As previously described, the machine learning engine 220 trains probabilistic models 230 associated with disease states. In the example shown in FIG. 6, the probabilistic models 230 (“tissue models”) are associated with non-cancer (healthy), breast cancer, and lung cancer. The analytics system 200 processes one or more cfDNA and/or tumor samples to obtain fragments and uses the probabilistic models 230 to assign a value to the fragments associated with non-cancer (healthy), breast cancer, and lung cancer. The analytics system 200 can use information from sequence reads from the cfDNA and/or tumor samples to identify features for a classifier. In some embodiments, the analytics system 200 can obtain and assign fragments from each window of a partitioned referenced genome, as shown in FIG. 5. The analytics system 200 aggregates the fragments from the windows to sequence for determining features for the classifier.

In step 140, the analytics system 200 identifies features by determining a count of the sequence reads having a value exceeding a threshold value. In embodiments where the value is based on the log-likelihood ratio R, the threshold value is a threshold ratio. The analytics system 200 can identify features using multiple tiers of threshold values. For example, the tiers include threshold values of 1, 2, 3, 4, 5, 6, 7, 8, and 9. Each tier indicates a varying threshold that a fragment (from which the sequence reads were generated) more likely originated from a sample associated with a disease state than from a healthy sample. The analytics system 200 can use the threshold value to determine counts of outlier fragments, which can be used as features.

By filtering with a threshold value, the analytics system 200 can consider certain fragments as outliers because the fragments are unlikely to be present in healthy samples. Accordingly, outlier fragments can be considered to be more likely associated with (e.g., originating from) a disease state or a cancer sample. The number of features can vary between different tiers. In other embodiments, the analytics system 200 uses a different number of tiers or other threshold values. In other embodiments, the analytics system 200 can filter fragments using other methods or scoring such as p-values. In some embodiments, the analytics system 200 calculates a p-value for a methylation state vector describing a probability of observing that methylation state vector or other methylation state vectors even less probable in a healthy control group. To determine a fragment to be anomalously methylated, the processing system 200 uses a healthy control group with a majority of fragments that are normally methylated (see, e.g., U.S. patent application Ser. No. 16/352,602, entitled “Anomalous Fragment Detection and Classification,” and filed Mar. 13, 2019, incorporated herein in reference to its entirety).

The analytics system 200 can repeat steps 130 to 140 for each probabilistic model trained in step 120. As a result, the processing system 200 can identify features for one or more disease states associated with the probabilistic models. In the example shown in FIG. 6, the analytics system 200 identifies one or more features for breast cancer and lung cancer.

In some embodiments, the analytics system 200 ranks the identified features based on measures of the features in distinguishing between different disease states. For instance, a feature is informative if the feature can distinguish a certain type of cancer from other types of cancer or healthy samples. The analytics system 200 can use mutual information to determine the measure of information content of a feature in distinguishing between two disease states. For each pair of distinct disease states, the analytics system 200 can designate one disease state, e.g., cancer type A, as a positive type and the other disease state, e.g., cancer type B, as a negative type.

The mutual information can be calculated using the estimated fraction of samples of the positive type and negative type (e.g., cancer types A and B) for which the feature is expected to be nonzero in a resulting assay. For instance, if a feature occurs frequently in healthy cfDNA, the analytics system 200 determines the feature is unlikely to occur frequently in cfDNA associated with various types of cancer. Consequently, the feature can be a weak measure in distinguishing between disease states. In calculating mutual information I, the variable Xis a certain feature (e.g., binary) and variable Y represents a disease state, e.g., cancer type A or B:

$$I ( X ; Y ) = {\sum }_{y ∈ Y}^{ } {\sum }_{x ∈ X}^{ } p ( x , y ) log log ( \frac{p ( x , y )}{p ( x ) p ( y )} ) I \approx \frac{1}{2} ( p ( 1 \vert A ) \cdot log ( \frac{p ( 1 \vert A )}{\frac{1}{2} ( p ( 1 \vert A ) + p ( 1 \vert B ) )} ) + p ( 1 \vert B ) \cdot log ( \frac{p ( 1 \vert B )}{\frac{1}{2} ( p ( 1 \vert A ) p ( 1 \vert B ) )} ) ) p ( 1 \vert A ) = {f}_{A} + {f}_{H} - {f}_{H} {f}_{A}$$

The joint probability mass function of X and Y is p(x, y) and the marginal probability mass functions are p(x) and p(y). The analytics system 200 can assume that feature absence is uninformative and either disease state is equally likely apriori, for example, p(Y=A)=p(Y=B)=0.5. The probability of observing (e.g., in cfDNA) a given binary feature of cancer type A is represented by p(1|A), where f_{A} is the probability of observing the feature in ctDNA samples from tumor (or high-signal cfDNA samples) associated with cancer type A, and f_{H} is the probability of observing the feature in a healthy or non-cancer cfDNA sample.

In some embodiments, the value of f_{A} is estimated by the fraction of cancer patients whose cfDNA would be expected to include a non-zero feature value. When the training data for cancer type A consists of cfDNA samples, this fraction can be estimated as simply the fraction of the cfDNA samples in which the feature is observed. When the training data includes tumor samples, a correction can be applied to account for the lower fraction of tumor-derived fragments in cfDNA compared to a tumor. For N fragments in a tumor sample determined to have a value greater than a threshold value (e.g., from step 140), the processing system 200 calculates a chance r of detecting each of those fragments in cfDNA from that patient as:

$$r = \frac{cfDNA sequencing depth \times cfDNA tumor fraction}{tumor sequencing depth}$$

The probability of observing at least one fragment in cfDNA from that patient can then be calculated as, p(N_{cfDNA}>0)=1−(1−r)^{N}. To estimate f_{A}, p(N_{cfDNA}>0) can be averaged across all training samples of cancer type A, where that probability is assigned as 1 for cfDNA samples that have the feature, 0 for cfDNA samples that lack the feature, and 1−(1−r)^{N} for tumor samples. In some embodiments, the estimates are based on predetermined assumed values for tumor fraction in the cfDNA of an early-stage cancer patient (e.g., 0.1%), cfDNA sequencing depth in the final assay to be applied to patients (e.g., 1000×), and the tumor sequencing depth (e.g., 25×). To estimate f_{H}, the analytics system 200 uses a fraction of positive samples to determine how many additional samples would result in a positive detection classification at greater sequencing depth.

### Classification

In step 150, the analytics system 200 generates a classifier using the features. The classifier is trained to predict, for an input sequence read from a test sample of a test subject, a tissue of origin associated with a disease state. The analytics system 200 can select a predetermined number (e.g., 1024) of top ranking features for each pair of disease states for training the classifier, e.g., based on the mutual information calculations or another calculated measure. The predetermined number can be treated as a hyperparameter selected based on performance in cross-validation. The analytics system 200 can also select features from regions of a reference genome determined to be more informative in distinguishing between the pair of disease states. In various embodiments, the analytics system 200 keeps the best performing tier for each region and for each cancer type pair (including non-cancer as a negative type).

In some embodiments, the analytics system 200 trains the classifier by inputting sets of training samples with their feature vectors into the classifier and adjusting classification parameters so that a function of the classifier accurately relates the training feature vectors to their corresponding label. The analytics system 200 can group the training samples into sets of one or more training samples for iterative batch training of the classifier. After inputting all sets of training samples including their training feature vectors and adjusting the classification parameters, the classifier can be sufficiently trained to label test samples according to their feature vector within some margin of error. The analytics system 200 can train the classifier according to any one of a number of methods, for example, L1-regularized logistic regression or L2-regularized logistic regression (e.g., with a log-loss function), generalized linear model (GLM), random forest, multinomial logistic regression, multilayer perceptron, support vector machine, neural net, or any other suitable machine learning technique.

In various embodiments, the analytics system 200 transforms feature values by binarization. In particular, feature values greater than 0 are set to 1, such that feature values are either 0 or 1 (indicating presence or absence of a disease state). In other embodiments, a smoothing function can be implemented (e.g., to provide more granular values) instead of binarization to 0 or 1.

In various embodiments, the analytics system 200 trains a multinomial logistic regression classifier on the training data for a fold and generates predictions for the held-out data. For each of the K folds, the analytics system 200 trains one logistic regression for each combination of hyperparameters. An example hyperparameter is the L2 penalty, i.e., a form of regularization applied to the weights of the logistic regression. Another example hyperparameter is the topK, i.e., the number of high-ranking regions to keep for each tissue type pair (including non-cancer). For instance, where topK=16, the analytics system 200 keeps the top 16 regions per tissue type pair, as ranked by the mutual information procedure described herein. By following this procedure, the analytics system 200 can generate a prediction for each sample in the training set while ensuring that classifiers are not trained on the data for which predictions are generated.

In various embodiments, for each set of hyperparameters, the analytics system 200 evaluates performance on the cross-validated predictions of the full training set, and the analytics system 200 selects the set of hyperparameters with the best performance for retraining on the full training set. Performance can be determined based on a log-loss metric. The analytics system 200 can calculate log-loss by taking the negative logarithm of the prediction for the correct label for each sample, and then summing over samples. For instance, a perfect prediction of 1.0 for the correct label would result in a log-loss of 0 (lower is more accurate). To generate predictions for a new sample, the analytics system 200 can calculate feature values using the method described above, but restricted to features (region/positive class combinations) selected under the chosen topK value. The analytics system 200 can use the generated features to create a prediction using the trained logistic regression model.

In an optional step 160, the analytics system 200 applies the classifier to predict a tissue of origin of a test sample, where the tissue of origin is associated with one of the disease states. In some embodiments, the classifier can return a prediction or likelihood for more than one disease state or tissue of origin. For example, the classifier can return a prediction that a test sample has a 65% likelihood of having a breast cancer tissue of origin, a 25% likelihood of having a lung cancer tissue of origin, and a 10% likelihood of having a healthy tissue of origin. The analytics system 200 can further process the prediction values to generate a single disease state determination.

### Multilayer Perceptron Model

In some embodiments, a multilayer perceptron model (“MLP”) can be used as an alternative to logistic regression for classification. As with the logistic regression based classifier, the MLP classifier can be a single multi-class classifier for both detecting cancer and determining a cancer tissue of origin (TOO) or cancer type. For example, the multi-class classifier can be trained to distinguish two or more, three or more, five or more, ten or more, fifteen or more, or twenty or more different types of cancer. In one embodiment, the multi-class cancer MLP model can also include a class label for non-cancer, and cancer detection can be determined (e.g., as 1-non-cancer). In another embodiment, the multilayer perceptron model can be a two-stage classifier having a first stage for binary classification (e.g., cancer or non-cancer), and a second stage multilayer perceptron model for multi-class classification (e.g., TOO), e.g., with one or more hidden layer.

In one embodiment, the multilayer perceptron comprises a two-stage classifier: a first stage multilayer perceptron (MLP) binary classifier with no hidden layer; and a second stage multilayer perceptron (MLP) multi-class classifier with a single hidden layer. In one embodiment, samples determined to have cancer using the first stage classifier will subsequently be analyzed by the second stage classifier.

In the first stage of training, a binary (two-class) multilayer perceptron model with no hidden layers for detecting the presence of cancer can be trained to discriminate cancer samples (regardless of TOO) from non-cancer. For each sample, the binary classifier outputs a prediction score indicating the likelihood of a presence or absence of cancer.

In the second stage of training, a parallel multi-class multilayer perceptron model for determining cancer type or cancer tissue of origin can be trained. In one embodiment, only cancer samples that received a score above a cutoff threshold (e.g., the 95th percentile of the non-cancer samples in the first stage classifier) can be included in the training of this multi-class MLP classifier. For each cancer sample used in training and testing, the multi-class MLP classifier outputs prediction values for the cancer types being classified, where each prediction value is a likelihood that the given sample has a certain cancer type. For example, the cancer classifier can return a cancer prediction for a test sample including a prediction score for breast cancer, a prediction score for lung cancer, and/or a prediction score for no cancer.

### Circulating Cell-Free Genome Atlas Study

In various embodiments, each predictive cancer model is trained using a set of training data derived from a training subset of patients of a circulating cell-free genome atlas (CCGA) study (See Clinical Trial.gov Identifier: NCT02889978) and then subsequently tested using a set of testing or validation data derived from a testing or validation subset of patients from the CCGA study.

The predictive cancer models described herein were trained using a plurality of known cancer types from the circulating cell-free genome atlas (CCGA) study. The CCGA sample set included the following cancer types: breast, lung, prostate, colorectal, renal, uterine, pancreas, esophageal, lymphoma, head and neck, ovarian, hepatobiliary, melanoma, cervical, multiple myeloma, leukemia, thyroid, bladder, gastric, and anorectal. As such, a model can be a multi-cancer model (or a multi-cancer classifier) for detecting of one or more, two or more, three or more, four or more, five or more, ten or more, or 20 or more different types of cancer.

Predictive cancer models can be trained using a refined set of training data derived from a first subset of patients of the CCGA study and then subsequently tested using a refined set of testing data derived from a second subset of patients from the CCGA study.

Cancer Assay Panel

In various embodiments, the predictive cancer models described herein use samples enriched using a cancer assay panel comprising a plurality of probes or a plurality of probe pairs. A number of targeted cancer assay panels are known in the art, for example, as describe in WO 2019/195268 filed Apr. 2, 2019, PCT/US2019/053509 filed Sep. 27, 2019 and PCT/US2020/015082 filed Jan. 24, 2020 (which are incorporated herein by reference in their entirety). For example, in some embodiments, the cancer assay panel can be designed to include a plurality of probes (or probe pairs) that can capture fragments that can together provide information relevant to diagnosis of cancer. In some embodiments, a panel includes at least 50, 100, 500, 1,000, 2,000, 2,500, 5,000, 6,000, 7,500, 10,000, 15,000, 20,000, 25,000, or 50,000 pairs of probes. In other embodiments, a panel includes at least 500, 1,000, 2,000, 5,000, 10,000, 12,000, 15,000, 20,000, 30,000, 40,000, 50,000, or 100,000 probes. The plurality of probes together can comprise at least 0.1 million, 0.2 million, 0.4 million, 0.6 million, 0.8 million, 1 million, 2 million, 3 million, 4 million, 5 million, 6 million, 7 million, 8 million, 9 million, or 10 million nucleotides. The probes (or probe pairs) are specifically designed to target one or more genomic regions differentially methylated in cancer and non-cancer samples. The target genomic regions can be selected to maximize classification accuracy, subject to a size budget (which is determined by sequencing budget and desired depth of sequencing).

Samples enriched using a cancer assay panel can be subject to targeted sequencing. Samples enriched using the cancer assay panel can be used to detect the presence or absence of cancer generally and/or provide a cancer classification such as cancer type, stage of cancer such as I, II, III, or IV, or provide the tissue of origin where the cancer is believed to originate. Depending on the purpose, a panel can include probes (or probe pairs) targeting genomic regions differentially methylated between general cancerous (pan-cancer) samples and non-cancerous samples, or only in cancerous samples with a specific cancer type (e.g., lung cancer-specific targets). Specifically, a cancer assay panel is designed based on bisulfite sequencing data generated from the cell-free DNA (cfDNA) or genomic DNA (gDNA) from cancer and/or non-cancer individuals.

In some embodiments, the cancer assay panel designed by methods provided herein comprises at least 1,000 pairs of probes, each pair of which comprises two probes configured to overlap each other by an overlapping sequence comprising a 30-nucleotide fragment. The 30-nucleotide fragment comprises at least five CpG sites, wherein at least 80% of the at least five CpG sites are either CpG or UpG. The 30-nucleotide fragment is configured to bind to one or more genomic regions in cancerous samples, wherein the one or more genomic regions have at least five methylation sites with an abnormal methylation pattern. Another cancer assay panel comprises at least 2,000 probes, each of which is designed as a hybridization probe complimentary to one or more genomic regions. Each of the genomic regions is selected based on the criteria that it comprises (i) at least 30 nucleotides, and (ii) at least five methylation sites, wherein the at least five methylation sites have an abnormal methylation pattern and are either hypomethylated or hypermethylated.

Each of the probes (or probe pairs) is designed to target one or more target genomic regions. The target genomic regions are selected based on several criteria designed to increase selective enriching of relevant cfDNA fragments while decreasing noise and non-specific bindings. For example, a panel can include probes that can selectively bind and enrich cfDNA fragments that are differentially methylated in cancerous samples. In this case, sequencing of the enriched fragments can provide information relevant to diagnosis of cancer. Furthermore, the probes can be designed to target genomic regions that are determined to have an abnormal methylation pattern and/or hypermethylation or hypomethylation patterns to provide additional selectivity and specificity of the detection. For example, genomic regions can be selected when the genomic regions have a methylation pattern with a low p-value according to a Markov model trained on a set of non-cancerous samples, that additionally cover at least 5 CpG's, 90% of which are either methylated or unmethylated. In other embodiments, genomic regions can be selected utilizing mixture models, as described herein.

Each of the probes (or probe pairs) can target genomic regions comprising at least 25 bp, 30 bp, 35 bp, 40 bp, 45 bp, 50 bp, 60 bp, 70 bp, 80 bp, or 90 bp. The genomic regions can be selected by containing less than 20, 15, 10, 8, or 6 methylation sites. The genomic regions can be selected when at least 80, 85, 90, 92, 95, or 98% of the at least five methylation (e.g., CpG) sites are either methylated or unmethylated in non-cancerous or cancerous samples.

Genomic regions can be further filtered to select only those that are likely to be informative based on their methylation patterns, for example, CpG sites that are differentially methylated between cancerous and non-cancerous samples (e.g., abnormally methylated or unmethylated in cancer versus non-cancer). For the selection, calculation can be performed with respect to each CpG site. In some embodiments, a first count is determined that is the number of cancer-containing samples (cancer count) that include a fragment overlapping that CpG, and a second count is determined that is the number of total samples containing fragments overlapping that CpG (total). Genomic regions can be selected based on criteria positively correlated to the number of cancer-containing samples (cancer count) that include a fragment overlapping that CpG, and inversely correlated with the number of total samples containing fragments overlapping that CpG (total).

In one embodiment, the number of non-cancerous samples (n_{non-cancer}) and the number of cancerous samples (n_{cancer}) having a fragment overlapping a CpG site are counted. Then the probability that a sample is cancer is estimated, for example as (n_{cancer}+1)/(n_{cancer}+n_{non-cancer}+2). CpG sites by this metric are ranked and greedily added to a panel until the panel size budget is exhausted.

Depending on whether the assay is intended to be a pan-cancer assay or a single-cancer assay, or depending on what kind of flexibility is desired when picking which CpG sites are contributing to the panel, which samples are used for cancer-count can vary. A panel for diagnosing a specific cancer type (e.g., TOO) can be designed using a similar process. In this embodiment, for each cancer type, and for each CpG site, the information gain is computed to determine whether to include a probe targeting that CpG site. The information gain is computed for samples with a given cancer type compared to all other samples. For example, two random variables, “AF” and “CT”. “AF” is a binary variable that indicates whether there is an abnormal fragment overlapping a particular CpG site in a particular sample (yes or no). “CT” is a binary random variable indicating whether the cancer is of a particular type (e.g., lung cancer or cancer other than lung). One can compute the mutual information with respect to “CT” given “AF.” That is, how many bits of information about the cancer type (lung vs. non-lung in the example) are gained if one knows whether there is an anomalous fragment overlapping a particular CpG site. This can be used to rank CpG's based on how specific they are for a particular cancer type (e.g., TOO). This procedure is repeated for a plurality of cancer types. For example, if a particular region is commonly differentially methylated only in lung cancer (and not other cancer types or non-cancer), CpG's in that region would tend to have high information gains for lung cancer. For each cancer type, CpG sites ranked by this information gain metric, and then greedily added to a panel until the size budget for that cancer type was exhausted.

Further filtration can be performed to select target genomic regions that have off-target genomic regions less than a threshold value. For example, a genomic region is selected only when there are less than 15, 10 or 8 off-target genomic regions. In other cases, filtration is performed to remove genomic regions when the sequence of the target genomic regions appears more than 5, 10, 15, 20, 25, or 30 times in a genome. Further filtration can be performed to select target genomic regions when a sequence, 90%, 95%, 98% or 99% homologous to the target genomic regions, appear less than 15, 10 or 8 times in a genome, or to remove target genomic regions when the sequence, 90%, 95%, 98% or 99% homologous to the target genomic regions, appear more than 5, 10, 15, 20, 25, or 30 times in a genome. This is for excluding repetitive probes that can pull down off-target fragments, which are not desired and can impact assay efficiency.

In some embodiments, fragment-probe overlap of at least 45 bp was demonstrated to be required to achieve a non-negligible amount of pulldown (though this number can be different depending on assay details). Furthermore, it has been suggested that more than a 10% mismatch rate between the probe and fragment sequences in the region of overlap is sufficient to greatly disrupt binding, and thus pulldown efficiency. Therefore, sequences that can align to the probe along at least 45 bp with at least a 90% match rate are candidates for off-target pulldown. Thus, in one embodiment, the number of such regions are scored. The best probes have a score of 1, meaning they match in only one place (the intended target region). Probes with a low score (say, less than 5 or 10) are accepted, but any probes above the score are discarded. Other cutoff values can be used for specific samples.

In various embodiments, the selected target genomic regions can be located in various positions in a genome, including but not limited to exons, introns, intergenic regions, and other parts. In some embodiments, probes targeting non-human genomic regions, such as those targeting viral genomic regions, can be added.

### Cancer Applications

In some embodiments, the methods, analytic systems and/or classifier of the present disclosure can be used to detect the presence (or absence) of cancer, monitor cancer progression or recurrence, monitor therapeutic response or effectiveness, determine a presence or monitor minimum residual disease (MRD), or any combination thereof. In some embodiments, the analytic systems and/or classifier can be used to identify the tissue or origin for a cancer. For instance, the systems and/or classifiers can be used to identify a cancer as of any of the following cancer types: head and neck cancer, liver/bileduct cancer, upper GI cancer, pancreatic/gallbladder cancer; colorectal cancer, ovarian cancer, lung cancer, multiple myeloma, lymphoid neoplasms, melanoma, sarcoma, breast cancer, and uterine cancer. For example, as described herein, a classifier can be used to generate a likelihood or probability score (e.g., from 0 to 100) that a sample feature vector is from a subject with cancer. In some embodiments, the probability score is compared to a threshold probability to determine whether or not the subject has cancer. In other embodiments, the likelihood or probability score can be assessed at different time points (e.g., before or after treatment) to monitor disease progression or to monitor treatment effectiveness (e.g., therapeutic efficacy). In still other embodiments, the likelihood or probability score can be used to make or influence a clinical decision (e.g., diagnosis of cancer, treatment selection, assessment of treatment effectiveness, etc.). For example, in one embodiment, if the likelihood or probability score exceeds a threshold, a physician can prescribe an appropriate treatment. In some embodiments, a test report can be generated to provide a patient with their test results, including, for example, a probability score that the patient has a disease state (e.g., cancer), a type of disease (e.g., a type of cancer), and/or a disease tissue of origin (e.g., a cancer tissue of origin).

### Early Detection of Cancer

In some embodiments, the methods and/or classifier of the present disclosure are used to detect the presence or absence of cancer in a subject suspected of having cancer. For example, a classifier (as described herein) can be used to determine a likelihood or probability score that a sample feature vector is from a subject that has cancer.

In one embodiment, a probability score of greater than or equal to 60 can indicated that the subject has cancer. In still other embodiments, a probability score greater than or equal to 65, greater than or equal to 70, greater than or equal to 75, greater than or equal to 80, greater than or equal to 85, greater than or equal to 90, or greater than or equal to 95, indicated that the subject has cancer. In other embodiments, a probability score can indicate the severity of disease. For example, a probability score of 80 may indicate a more severe form, or later stage, of cancer compared to a score below 80 (e.g., a score of 70). Similarly, an increase in the probability score over time (e.g., at a second, later time point) can indicate disease progression or a decrease in the probability score over time (e.g., at a second, later time point) can indicate successful treatment.

In another embodiment, a cancer log-odds ratio can be calculated for a test subject by taking the log of a ratio of a probability of being cancerous over a probability of being non-cancerous (i.e., one minus the probability of being cancerous), as described herein. In accordance with this embodiment, a cancer log-odds ratio greater than 1 can indicate that the subject has cancer. In still other embodiments, a cancer log-odds ratio greater than 1.2, greater than 1.3, greater than 1.4, greater than 1.5, greater than 1.7, greater than 2, greater than 2.5, greater than 3, greater than 3.5, or greater than 4, indicated that the subject has cancer. In other embodiments, a cancer log-odds ratio can indicate the severity of disease. For example, a cancer log-odds ratio greater than 2 may indicate a more severe form, or later stage, of cancer compared to a score below 2 (e.g., a score of 1). Similarly, an increase in the cancer log-odds ratio over time (e.g., at a second, later time point) can indicate disease progression or a decrease in the cancer log-odds ratio over time (e.g., at a second, later time point) can indicate successful treatment.

According to aspects of the disclosure, the methods and systems of the present disclosure can be trained to detect or classify multiple cancer indications. For example, the methods, systems and classifiers of the present disclosure can be used to detect the presence of one or more, two or more, three or more, five or more, or ten or more different types of cancer.

In some embodiments, the cancer is one or more of head and neck cancer, liver/bileduct cancer, upper GI cancer, pancreatic/gallbladder cancer; colorectal cancer, ovarian cancer, lung cancer, multiple myeloma, lymphoid neoplasms, melanoma, sarcoma, breast cancer, and uterine cancer.

### Cancer and Treatment Monitoring

In certain embodiments, the first time point is before a cancer treatment (e.g., before a resection surgery or a therapeutic intervention), and the second time point is after a cancer treatment (e.g., after a resection surgery or therapeutic intervention), and the method utilized to monitor the effectiveness of the treatment. For example, if the second likelihood or probability score decreases compared to the first likelihood or probability score, then the treatment is considered to have been successful. However, if the second likelihood or probability score increases compared to the first likelihood or probability score, then the treatment is considered to have not been successful. In other embodiments, both the first and second time points are before a cancer treatment (e.g., before a resection surgery or a therapeutic intervention). In still other embodiments, both the first and the second time points are after a cancer treatment (e.g., before a resection surgery or a therapeutic intervention) and the method is used to monitor the effectiveness of the treatment or loss of effectiveness of the treatment. In still other embodiments, cfDNA samples can be obtained from a cancer patient at a first and second time point and analyzed. e.g., to monitor cancer progression, to determine if a cancer is in remission (e.g., after treatment), to monitor or detect residual disease or recurrence of disease, or to monitor treatment (e.g., therapeutic) efficacy.

Those of skill in the art will readily appreciate that test samples can be obtained from a cancer patient over any desired set of time points and analyzed in accordance with the methods of the disclosure to monitor a cancer state in the patient. In some embodiments, the first and second time points are separated by an amount of time that ranges from about 15 minutes up to about 30 years, such as about 30 minutes, such as about 1, 2, 3, 4, 5, 6, 7, 8, 9, 10, 11, 12, 13, 14, 15, 16, 17, 18, 19, 20, 21, 22, 23, or about 24 hours, such as about 1, 2, 3, 4, 5, 10, 15, 20, 25 or about 30 days, or such as about 1, 2, 3, 4, 5, 6, 7, 8, 9, 10, 11, or 12 months, or such as about 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5 or about 30 years. In other embodiments, test samples can be obtained from the patient at least once every 3 months, at least once every 6 months, at least once a year, at least once every 2 years, at least once every 3 years, at least once every 4 years, or at least once every 5 years

### Treatment

In still another embodiment, information obtained from any method described herein (e.g., the likelihood or probability score) can be used to make or influence a clinical decision (e.g., diagnosis of cancer, treatment selection, assessment of treatment effectiveness, etc.). For example, in one embodiment, if the likelihood or probability score exceeds a threshold, a physician can prescribe an appropriate treatment (e.g., a resection surgery, radiation therapy, chemotherapy, and/or immunotherapy). In some embodiments, information such as a likelihood or probability score can be provided as a readout to a physician or subject.

A classifier (as described herein) can be used to determine a likelihood or probability score that a sample feature vector is from a subject that has cancer. In one embodiment, an appropriate treatment (e.g., resection surgery or therapeutic) is prescribed when the likelihood or probability exceeds a threshold. For example, in one embodiment, if the likelihood or probability score is greater than or equal to 60, one or more appropriate treatments are prescribed. In another embodiments, if the likelihood or probability score is greater than or equal to 65, greater than or equal to 70, greater than or equal to 75, greater than or equal to 80, greater than or equal to 85, greater than or equal to 90, or greater than or equal to 95, one or more appropriate treatments are prescribed. In other embodiments, a cancer log-odds ratio can indicate the effectiveness of a cancer treatment. For example, an increase in the cancer log-odds ratio over time (e.g., at a second, after treatment) can indicate that the treatment was not effective. Similarly, a decrease in the cancer log-odds ratio over time (e.g., at a second, after treatment) can indicate successful treatment. In another embodiment, if the cancer log-odds ratio is greater than 1, greater than 1.5, greater than 2, greater than 2.5, greater than 3, greater than 3.5, or greater than 4, one or more appropriate treatments are prescribed.

In some embodiments, the treatment is one or more cancer therapeutic agents selected from the group including a chemotherapy agent, a targeted cancer therapy agent, a differentiating therapy agent, a hormone therapy agent, and an immunotherapy agent. For example, the treatment can be one or more chemotherapy agents selected from the group including alkylating agents, antimetabolites, anthracyclines, anti-tumor antibiotics, cytoskeletal disruptors (taxans), topoisomerase inhibitors, mitotic inhibitors, corticosteroids, kinase inhibitors, nucleotide analogs, platinum-based agents and any combination thereof. In some embodiments, the treatment is one or more targeted cancer therapy agents selected from the group including signal transduction inhibitors (e.g. tyrosine kinase and growth factor receptor inhibitors), histone deacetylase (HDAC) inhibitors, retinoic receptor agonists, proteosome inhibitors, angiogenesis inhibitors, and monoclonal antibody conjugates. In some embodiments, the treatment is one or more differentiating therapy agents including retinoids, such as tretinoin, alitretinoin and bexarotene. In some embodiments, the treatment is one or more hormone therapy agents selected from the group including anti-estrogens, aromatase inhibitors, progestins, estrogens, anti-androgens, and GnRH agonists or analogs. In one embodiment, the treatment is one or more immunotherapy agents selected from the group comprising monoclonal antibody therapies such as rituximab (RITUXAN) and alemtuzumab (CAMPATH), non-specific immunotherapies and adjuvants, such as BCG, interleukin-2 (IL-2), and interferon-alfa, immunomodulating drugs, for instance, thalidomide and lenalidomide (REVLIMID). It is within the capabilities of a skilled physician or oncologist to select an appropriate cancer therapeutic agent based on characteristics such as the type of tumor, cancer stage, previous exposure to cancer treatment or therapeutic agent, and other characteristics of the cancer.

### EXAMPLES

### Example 1—Whole-Genome Bisulfite Sequencing (WGBS)

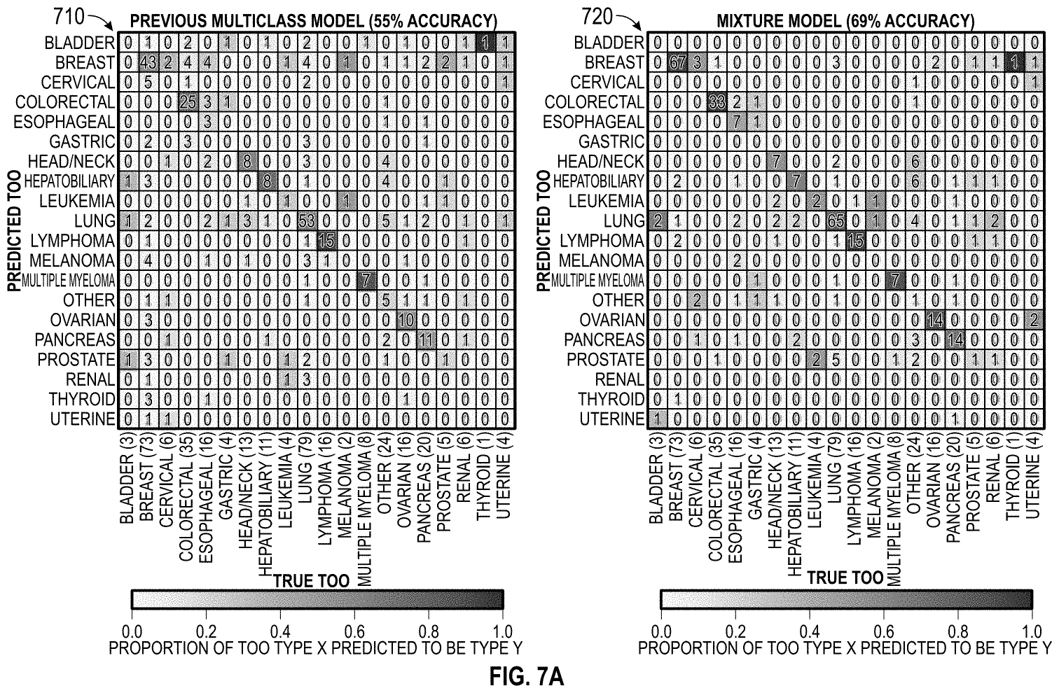

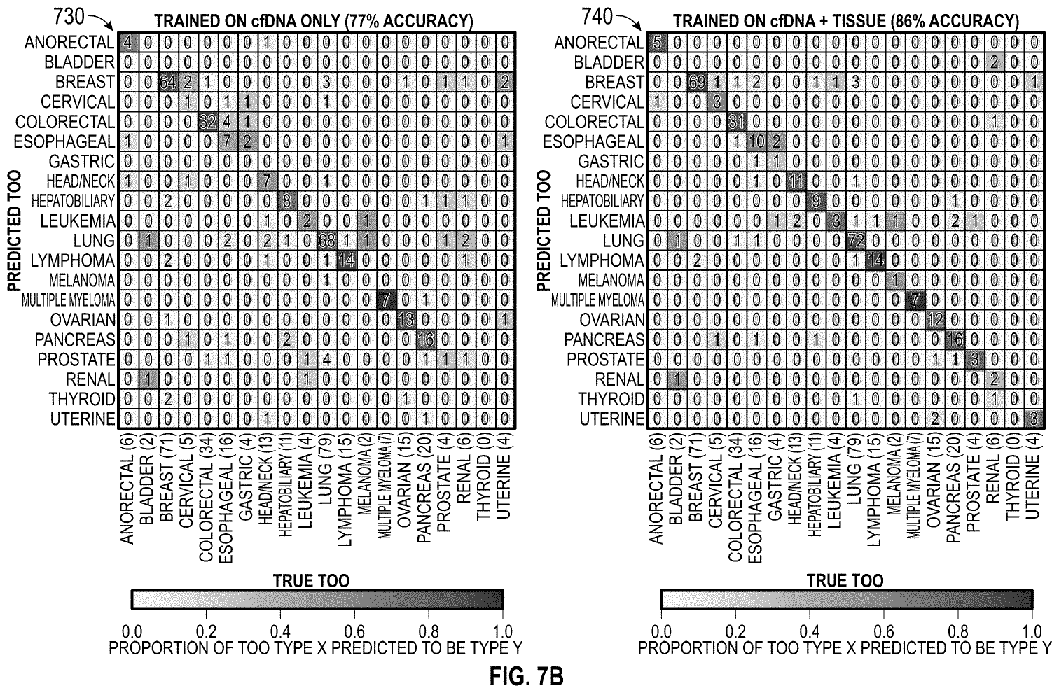

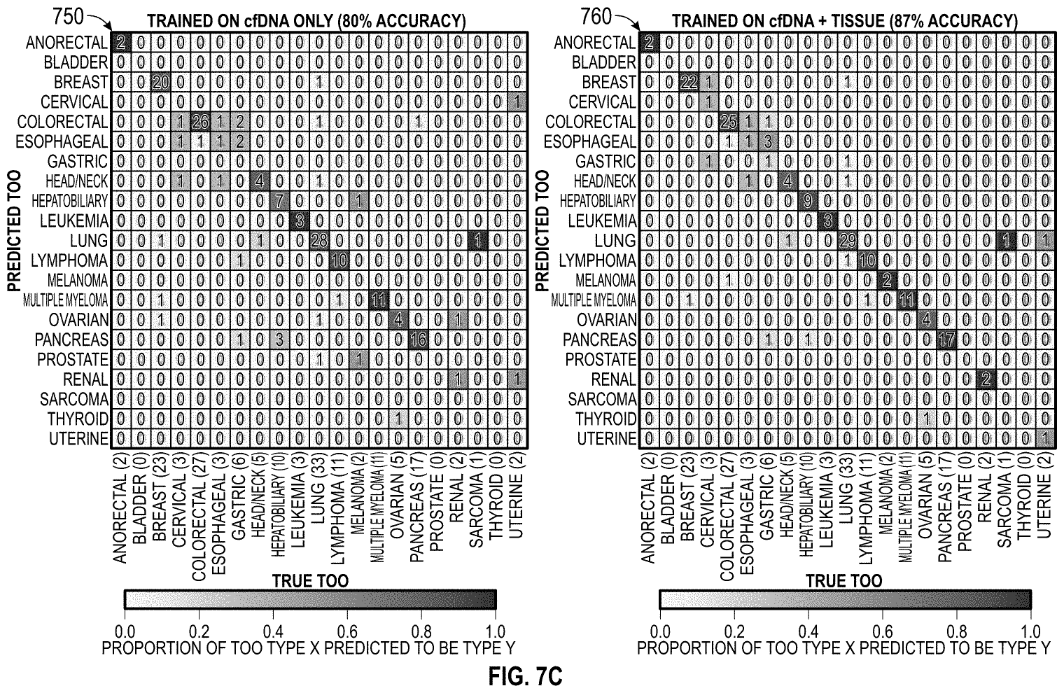

First CCGA substudy: The data shown in FIGS. 7A-C were obtained from a first CCGA substudy where training data blood samples (N=1785) were collected from individuals diagnosed with untreated cancer (including 20 tumor types and all stages of cancer) and healthy individuals with no cancer diagnosis (controls) for plasma cfDNA extraction. Another set of blood samples (N=1,010) were collected to be used for validation. Unless otherwise indicated, extracted cell-free DNA (cfDNA) and genomic DNA (gDNA) from the first CCGA substudy samples were subjected to a whole-genome bisulfite sequencing assay.

In the classification process, the analytics system 200 treats fragment methylation states as being drawn from a mixture of latent methylation patterns. The analytics system 200 assigns observed fragments a relative probability of originating from a particular cancer tissue of origin.

More specifically, as described herein, a probabilistic model was fit to the sequence reads derived from a plurality of regions (or windows) from each cancer type (and for non-cancer or healthy samples). In this case, a mixture model was used where each mixture component was an independent-sites model (in which methylation at each CpG is independent of methylation at other CpGs). Models were fit using maximum likelihood estimation to identify the set of parameters that maximize the total log-likelihood of all fragments derived from one cancer type (or non-cancer).

For each region, for each cancer type pair (including non-cancer as a negative type), the best performing tiers were used to train a multinomial logistic regression classifier. For each sample (regardless of label), in each region, for each cancer type, for each fragment, the log-likelihood ratio was calculated, as previously described, and for each of a set of “tier” values the number of fragments with R_{cancer type}>tier were quantified. Quantified reads for each of the tiers were binarized and used as features to train the classifier.

Finally, where indicated, to generate predictions for an unknown sample feature values were determined (as described above) and the generated features were used to create a cancer and/or tissue of origin prediction utilizing the trained multinomial logistic regression classifier.

Example confusion matrices: FIGS. 7A, 7B, and 7C include confusion matrices indicating accuracy of classifiers, according to various embodiments. In some embodiments, the analytics system 200 determines an accuracy of the classifier using a confusion matrix. The confusion matrix includes information describing a success rate for the classifier at identifying each of the disease states.

As shown in FIG. 7A, matrix 710 includes example performance of a classifier based on a multinomial model trained using a set of cfDNA samples (no tissue samples). Matrix 720 includes an example performance of a classifier based on a mixture model trained by the analytics system 200 using the same set of cfDNA samples. Scores along the diagonal of the matrices indicate correct predictions, that is, where the predicted tissue of origin for a fragment matches the true tissue of origin. In comparison to the classifier based on the multinomial model as a baseline, the classifier based on the mixture model has greater overall accuracy in predicting presence of the types of cancers shown in the matrices.

Samples of the training sets can be filtered based on one or more criteria (e.g., a particular specificity level). For example, the training sets include samples determined to have cancer based on a 98% specificity according to an m-score. The remaining (e.g., 2%) non-cancer samples that were (erroneously) identified as having cancer were excluded from being displayed in the confusion matrices for clarity.

As shown in FIG. 7B, matrix 730 includes an example performance of a classifier based on a mixture model trained using a cross-validation training set of cfDNA samples (no tissue samples). Matrix 740 includes an example performance of a classifier based on a mixture model trained using a cross-validation training set of cfDNA and tissue samples.

As shown in FIG. 7C, matrix 750 includes an example performance of a classifier based on a mixture model trained using a set of cfDNA samples (no tissue samples) from a clinical study titled Circulating Cell-free Genome Atlas Study (“CCGA”). Matrix 740 includes an example performance of a classifier based on a mixture model trained using a set of cfDNA and tissue samples from CCGA. The CCGA study was described with Clinical Trial.gov Identifier: NCT02889978 (https://www.clinicaltrials.gov/ct2/show/NCT02889978).

### Example 2—Classification of Cancer Using Targeted Bisulfite Sequencing from Early Breakout of the Second CCGA Substudy

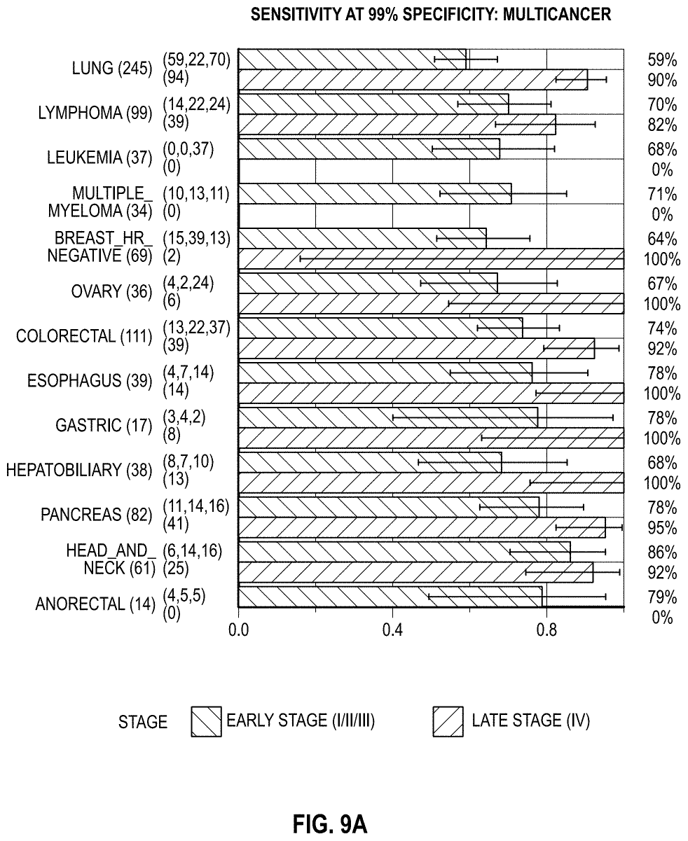

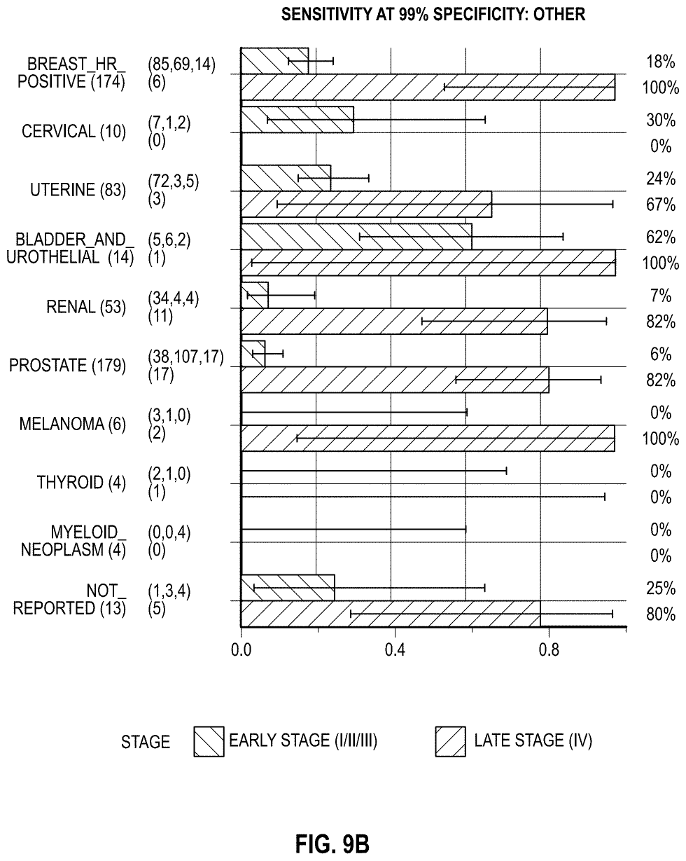

Second CCGA substudy: The data shown in FIGS. 9A-B, 10A-B, and 11 were obtained from an early breakout from the second CCGA sub-study where training data blood samples (N=3,132) were collected from individuals diagnosed with untreated cancer (including 20 tumor types and all stages of cancer) and healthy individuals with no cancer diagnosis (controls) for plasma cfDNA extraction. Another set of blood samples (N=1,354) were collected to be used for validation. In some embodiments, where indicated, the training set also included training data from tissue samples (i.e., gDNA). To determine the analysis population, the training data blood samples were filtered based on several factors. For example, 105 samples were excluded as clinically unlocked; 11 samples were excluded based on eligibility criteria; 58 samples were excluded for unconfirmed cancer or treatment status (not evaluable); 4 non-processed samples and 72 non-evaluable assays were excluded (not analyzable); and 581 samples were reserved for future analysis. As a result, the analysis population of 2,301 samples included 1,422 cancer samples and 879 non-cancer samples.

Participant demographics of individuals in the sub-study are shown below in Table 1.

TABLE 1

| | Cancer* | Non-Cancer |
|---|---|---|
| Total | 1,422 | 879 |
| Age, Mean ± SD | 62.0 ± 11.8 | 54.2 ± 13.6 |
| Age Group, n (%) | | |
| ≥50 years | 1220 (85.8) | 576 (65.5) |
| Sex, n (%) | | |
| Female | 712 (50.1) | 583 (66.3) |
| Race/Ethnicity (%) | | |
| White, Non-Hispanic | 1174 (82.6) | 713 (81.1) |
| African American | 97 (6.8) | 67 (7.6) |
| Hispanic, Asian, Other | 151 (10.6) | 99 (11.3) |
| Smoking Status, n (%) [symbol] | | |
| Never-smoker | 633 (45.3) | 495 (57.1) |
| Body Mass Index, n (%)^{‡} | | |
| Normal/Underweight | 381 (26.8) | 216 (24.6) |
| Overweight | 490 (34.5) | 309 (35.2) |
| Obese | 551 (38.7) | 352 (40.1) |
| Method of Dx, n (%) | | |
| Dx by Screening | 350 (24.6) | — |
| Clinical Stage, n (%)^{§} | | |
| I | 398 (28.0) | — |
| II | 366 (25.7) | — |
| III | 290 (20.4) | — |
| IV | 327 (23.0) | — |
| Non-informative/Missing^{¶} | 41 (2.9) | — |

Table 1: Participant demographics and stage distribution. Cancer and non-cancer groups were comparable with respect to age, race, sex, and body mass index (not shown).
*Includes anorectal, bladder, brain, breast, cervical, colorectal, esophageal, gastric, head and neck, hepatobiliary, lung, lymphoid neoplasm (chronic lymphocytic leukemia, lymphoma), multiple myeloma, myeloid neoplasm (acute myeloid leukemia, chronic myeloid leukemia), ovarian, pancreatic, prostate, renal, sarcoma, and uterine cancers. [symbol]
Excludes 38 participants missing smoking status information.
^{‡}Excludes two participants missing BMI values.
^{§}Invasive cancer only.
^{¶}Staging information not available.

To identify cancer-defining and tissue-defining methylation signals, the extracted cfDNA was subjected to a bisulfite sequencing assay targeting the most informative regions of the methylome, as identified from GRAIL's proprietary whole-genome bisulfite sequencing assay and methylation database.

We used a methylation database that interrogated genome-wide fragment-level methylation patterns across 811 cancer cell methylomes representing 21 tumor types (97% of SEER cancer incidence). To generate the methylation database of cancer-defining methylation signals, genomic DNA from formalin-fixed, paraffin-embedded (FFPE) tumor tissues and isolated cells from tumors were subjected to a whole-genome bisulfite sequencing assay. The methylation database was used for panel design and training to optimize performance of classifiers, as described herein. A large methylation sequence database of cancer and non-cancer was generated to enable target selection for a single test able to classify multiple cancers at high specificity and identify tissue of origin.

Target selection and panel design: Target genomic regions were selected using the methylation sequence database from the CCGA study, as described herein. Specifically, cfDNA sequences in the database were filtered based on p-value using a non-cancer distribution, and only fragments with p<0.001 were retained. The selected cfDNAs were further filtered to retain only those that were at least 90% methylated or 90% unmethylated. Next, for each CpG site in the selected fragments, the numbers of cancer samples or non-cancer samples were counted that include fragments overlapping that CpG site. Specifically, P (cancer|overlapping fragment) for each CpG was calculated and genomic sites with high P values were selected as general cancer targets. By design, the selected fragments had very low noise (i.e., few non-cancer fragments overlapping).

To find cancer type specific targets, similar selection processes were performed. CpG sites were ranked based on their information gain, comparing one cancer type to all other samples (i.e., non-cancer plus other cancer types).

Cancer assay panels comprising probes targeting the selected genomic regions were generated, as described herein. Specifically, the panels were designed to detect the presence of cancer generally (i.e., vs non-cancer) or a specific cancer type (e.g., TOO). The panels include probe set targeting each of the genomic regions selected.

Probes were designed to overlap any of the CpG sites included within the start/stop ranges of any of the targeted regions (e.g., anomalous fragments).

Classification: In the classification process, the analytics system 200 treats fragment methylation states as being drawn from a mixture of latent methylation patterns. The analytics system 200 assigns observed fragments a relative probability of originating from cancer. For tissue of origin classification, the analytics system 200 assigns observed fragments a relative probability of originating from a particular tissue. The analytics system 200 combines fragments characteristic of cancer and tissue of origin across targeted regions to classify cancer versus non-cancer and/or identify tissue of origin. For binary cancer classification, the analytics system 200 estimates sensitivity at 99% specificity.

More specifically, as described in an example above, a probabilistic model was fit to the sequence reads derived from a plurality of regions (or windows) from each cancer type (and for non-cancer or healthy samples), features identified, and a multinomial logistic regression classifier trained. To generate predictions for an unknown sample, feature values were determined (as described above) and the generated features were used to create a cancer and/or tissue of origin prediction utilizing the trained multinomial logistic regression classifier.

FIGS. 9A and 9B illustrate sensitivity of tissue of origin classifiers generated by methods described in the present disclosure. The sensitivity is reported at 99% specificity, and 95% confidence intervals are indicated. FIG. 9A illustrates model predictions for a pre-specified list of cancers. FIG. 9B illustrates model predictions for other cancers included in the CCGA study. Demographic information alone (baseline modeling) classified <5% of participants correctly. Overall sensitivity was 76.1% (95% CI: 73.1-78.9%) in a pre-specified list of cancers (anorectal, breast [HR-negative], colorectal, esophageal, gastric, head and neck, hepatobiliary, lung, lymphoid neoplasm [chronic lymphocytic leukemia, lymphoma], multiple myeloma, ovarian, pancreatic). Sensitivity was 68.8% (95% CI: 64.8-72.6%) in early stage (I-III) cancers in this cohort. Overall sensitivity was 55.1% (95% CI: 52.5-57.7%) across all cancer types and stages. In early stage (I-III) cancers, sensitivity was 43.8% (95% CI: 40.7-46.8%).

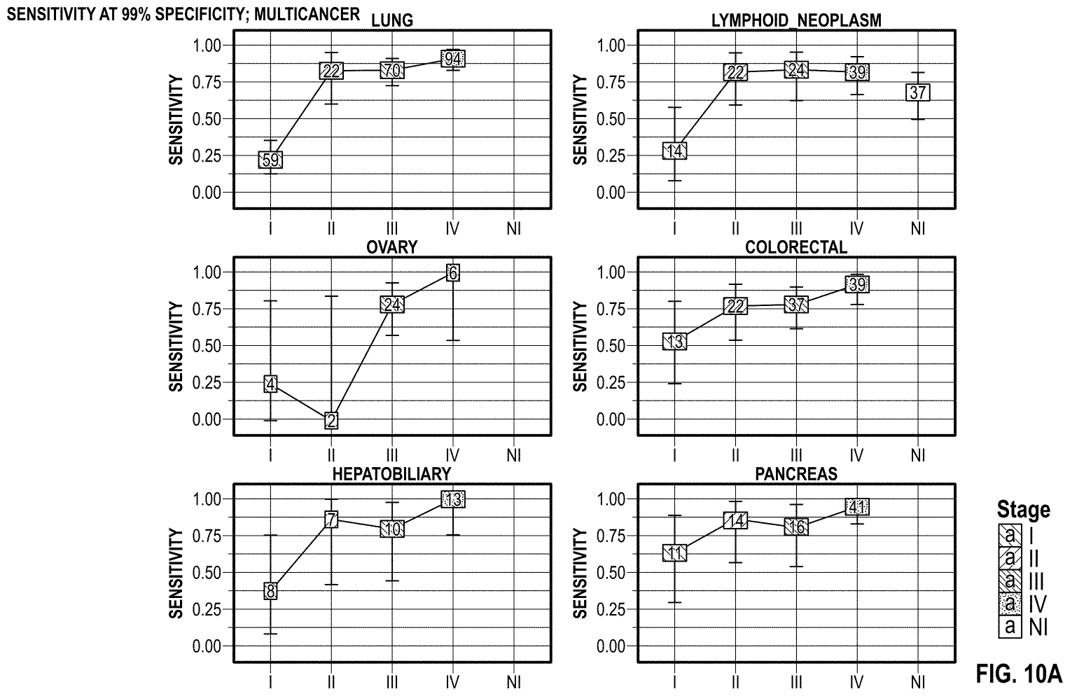

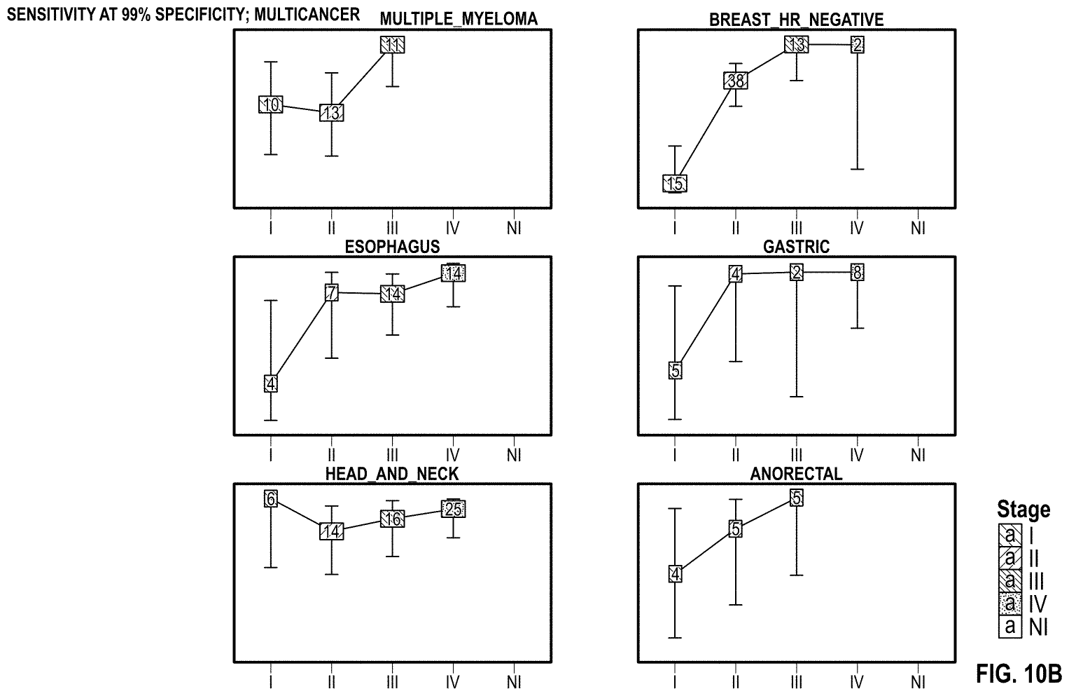

FIGS. 10A and 10B illustrate sensitivity of the tissue of origin classifiers at different cancer stages. Sensitivity by individual stage, as indicated in the legend, for the pre-specified cancers-of-interest in aggregate is reported at 99% specificity. Numbers within boxes represent the total number of samples included at each stage. 95% confidence intervals are indicated. “Lymphoid neoplasm” includes lymphoma (stages I-IV) and chronic lymphocytic leukemia (un-staged, included as “NI”).

FIG. 11 illustrates a performance grid representing the accuracy of tissue of origin localization. There is agreement between the true (x-axis) and predicted (y-axis) tissue of origin per sample using the tissue of origin classifier with the methylation database in stage I-IV samples. The gradient legend corresponds to the proportion of predicted tissue of origin (y-axis) which were correct (x-axis). The analysis showed that accuracy of tissue of origin localization (the fraction of all TOO predictions that were correct) was higher with the methylation database (p=0.0066). This was consistent in stage I-III predictions: 89.9% (384/427) as further demonstrated in Table 2.

TABLE 2

| | Without | With | | |
|---|---|---|---|---|
| | Methylation | Methylation | | |
| TOO Calls | Database | Database | Delta | P-value* |
| Correct | 642 | 663 | +21 | — |
| Indeterminate† | 74 | 72 | −2 | — |
| Incorrect | 47 | 49 | +2 | — |
| Cancer not | 659 | 638 | −21 | — |
| Detected‡ | | | | |
| Total | | | | p = 0.0066 |

Table 2: Tissue of origin performance improves when including the methylation database.
*P-value calculated using the Stuart-Maxwell test.
† Indeterminate calls were defined as samples detected as cancer but without a confident tissue of origin assignment.
‡ Samples not called by the tissue of origin analysis were classified as non-cancer.

### Example 3—Detection of Viral Cell-free Nucleic Acid Molecules and Classification of HPV-associated Cancers

Introduction: Human papillomaviruses (HPV) are a diverse group of viruses with a viral genome approximately 8.2 kb in length. HPV infections are extremely prevalent, with 80M Americans currently infected with some type of HPV and where 9/10 infections are transient (i.e., cleared within 2 years). Certain types of HPV can greatly increase risk of developing cancer, such as 70% of oropharyngeal cancers in the USA, >90% of cervical and anorectal cancers in the USA, and also cancers of the vagina, vulva, and penis. HPV 16 and HPV 18 account for the vast majority of HPV-driven cancer cases. In the present example, HPV fragments were pulled down using a targeted panel design (e.g., targeted panel design for binary cancer classification and multiclass cancer classification) with probes covering both HPV 16 and HPV 18 genomes. The targeted panel achieved a useful signal for classification and resolved TOO confusion in the HPV axis by greatly improving anorectal TOO accuracy, at little to no cost.

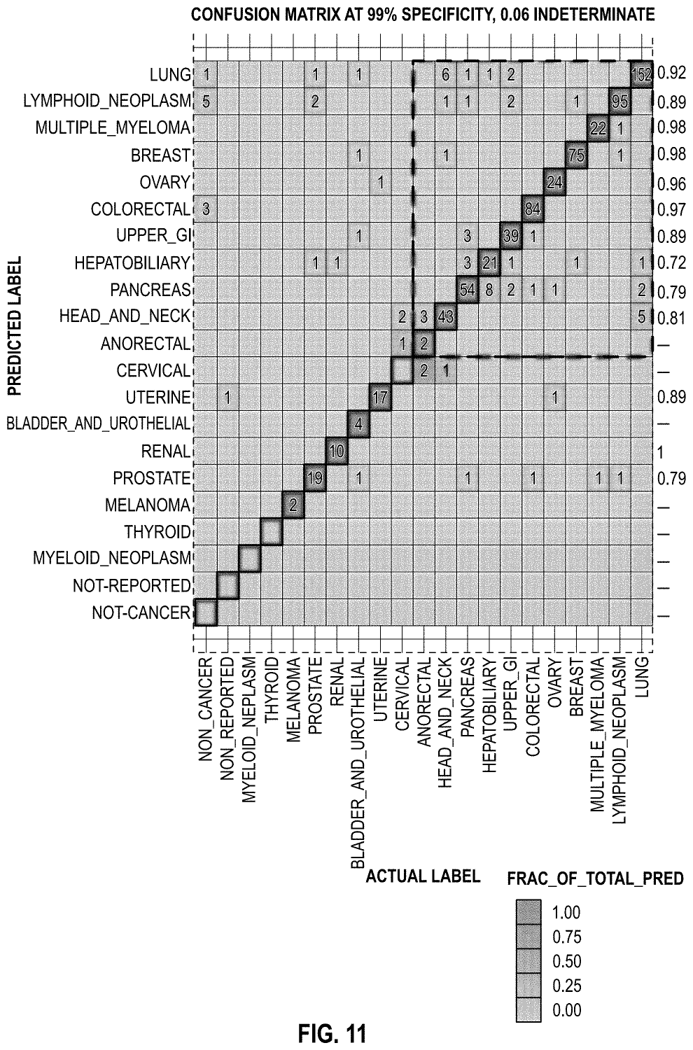

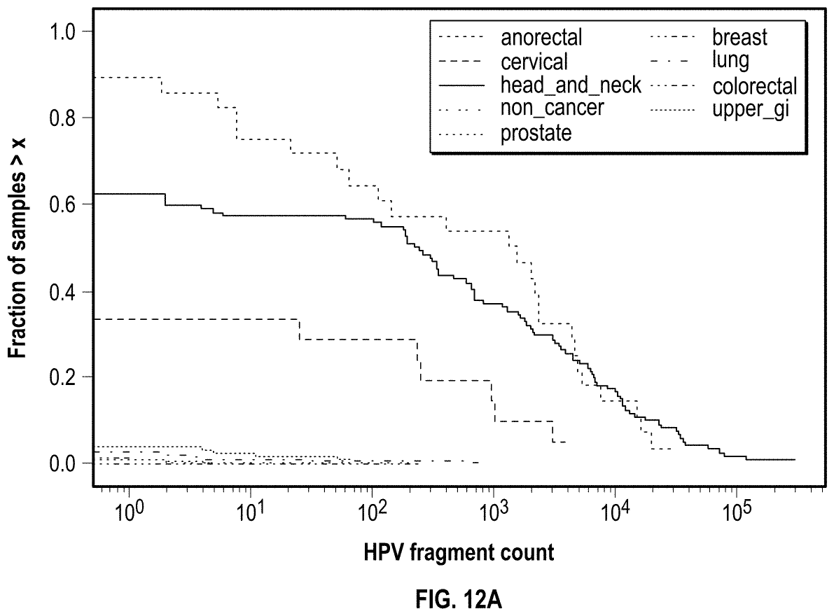

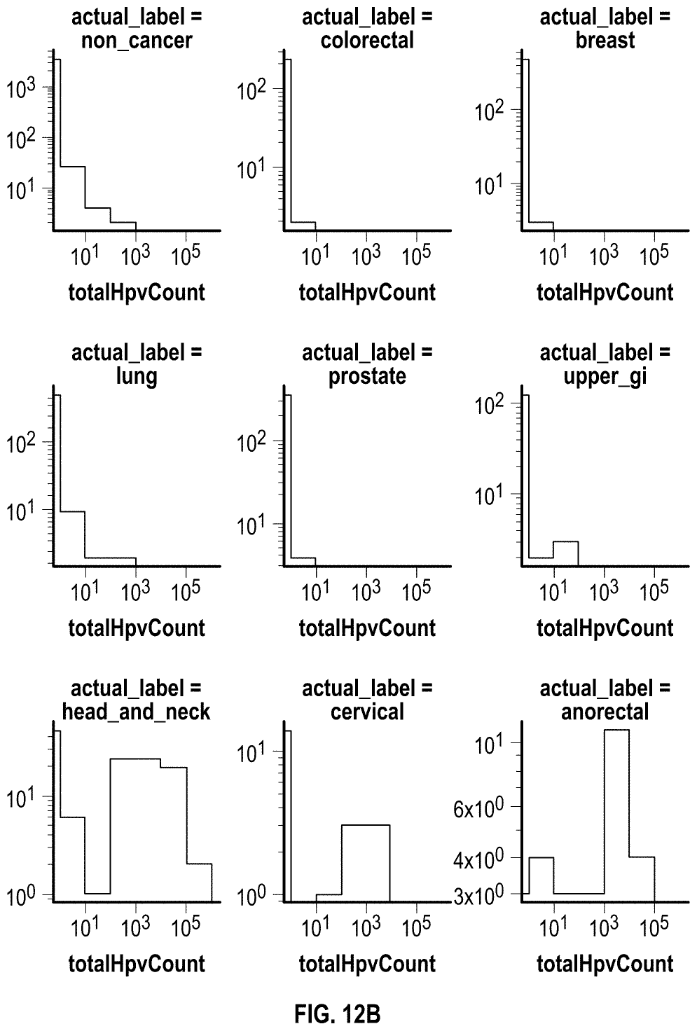

An initial observation from the dataset of this investigation showed that HPV fragments are very rare in non-HPV cancers. For instance, FIG. 12A illustrates a graph of HPV fragment count versus fraction of samples>X across various cancer types. As shown at FIG. 12, HPV fragments are noticeably more prevalent in HPV-associated cancers (e.g., anorectal, head and neck, and cervical cancers) and much less prevalent in non-HPV-associated cancers (e.g., prostate, breast, lung, colorectal, upper GI, and non-cancers). Further, approximately 99.2% of non-cancers have 0 HPV fragments. In the bar charts at FIG. 12B, which compares HPV fragment counts across various cancer types, this rarity of HPV fragments in non-HPV cancers is also shown. For instance, the top two rows of bar charts at FIG. 12B shows how few HPV fragments are present in non-HPV-associated cancer cfDNA samples, such as colorectal, breast, lung, prostate, and upper GI cancer samples, as well as non-cancer samples. On the other hand, the bottom third row of bar charts at FIG. 12B shows a much higher presence of HPV fragments in HPV-associated cancer cfDNA samples, such as head and neck, cervical, and anorectal cancer samples. It is noted that FIGS. 12A-B show evaluable cfDNA samples only and include HPV fragment counts that are summed across HPV 16 and HPV 18.

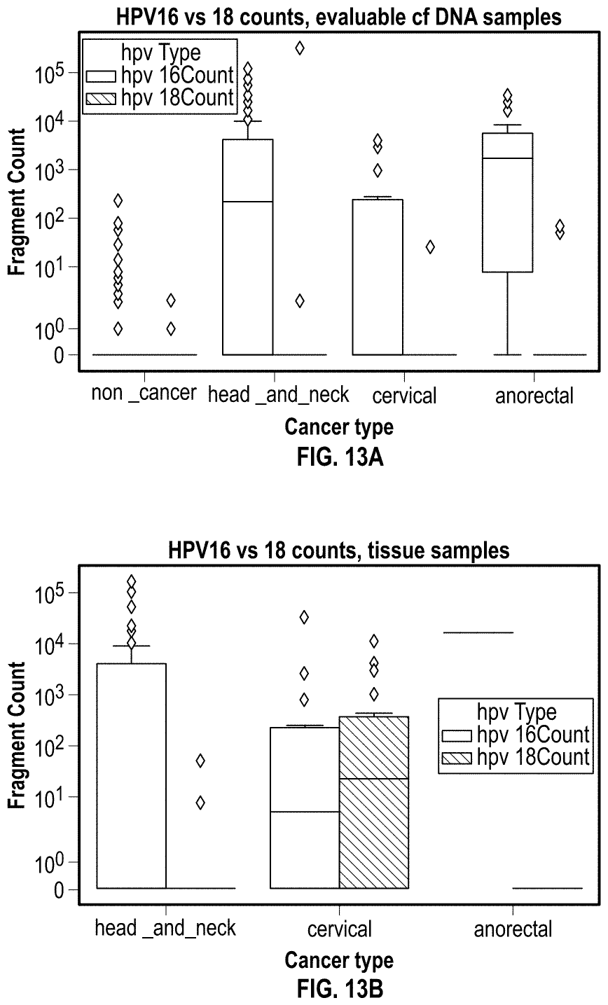

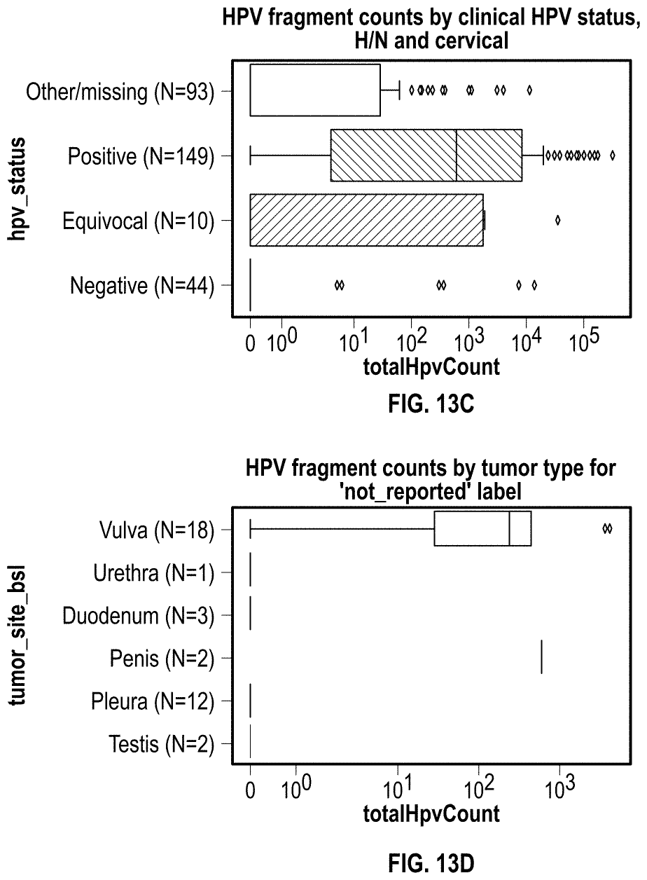

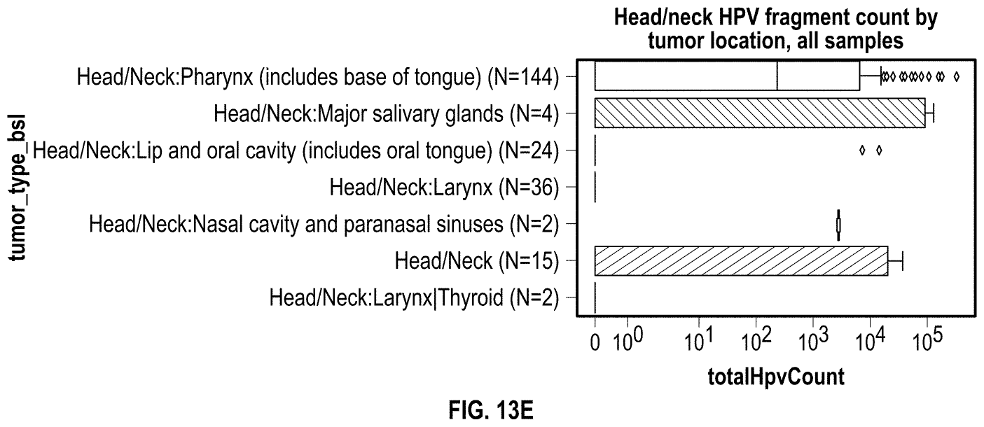

FIGS. 13A-13D demonstrate that HPV fragment pulldown in CCGA2, in accordance with various embodiments described herein, is consistent with expected biology. For instance, FIG. 13A illustrates a bar chart showing HPV 16 and HPV 18 fragment counts in evaluable cfDNA samples for various cancer type classes, including non-cancer, head and neck, cervical, and anorectal. FIG. 13B illustrates a bar chart showing HPV 16 and HPV 18 fragment counts in tissue samples for various cancer types, including head and neck, cervical, and anorectal. Both of FIGS. 13A-B illustrate that HPV 18 is much rarer than HPV 16, and that HPV 18 is largely restricted to cervical cancers.

FIG. 13C illustrates a bar chart showing HPV fragment counts by clinical HPV status for head and neck and cervical cancer samples across different HPV statuses, such as positive, equivocal, negative, and other/missing status. FIG. 13D illustrates a bar chart showing HPV fragment counts by tumor type for not reported cancer samples, such as vulva, urethra, duodenum, penis, pleura, and testis. As shown at FIGS. 13C-D, HPV fragment counts are largely concordant with clinical status.

FIG. 13E illustrates a bar chart showing head/neck HPV fragment count by tumor location across all samples, the tumor locations including pharynx (includes base of tongue), major salivary glands, lip and oral cavity (includes tongue), larynx, nasal cavity and paranasal sinuses, head/neck, and larynx/thyroid. As shown at FIG. 13E, head/neck samples with HPV fragments are largely restricted to the pharynx. All of the larynx samples have 0 HPV fragments.

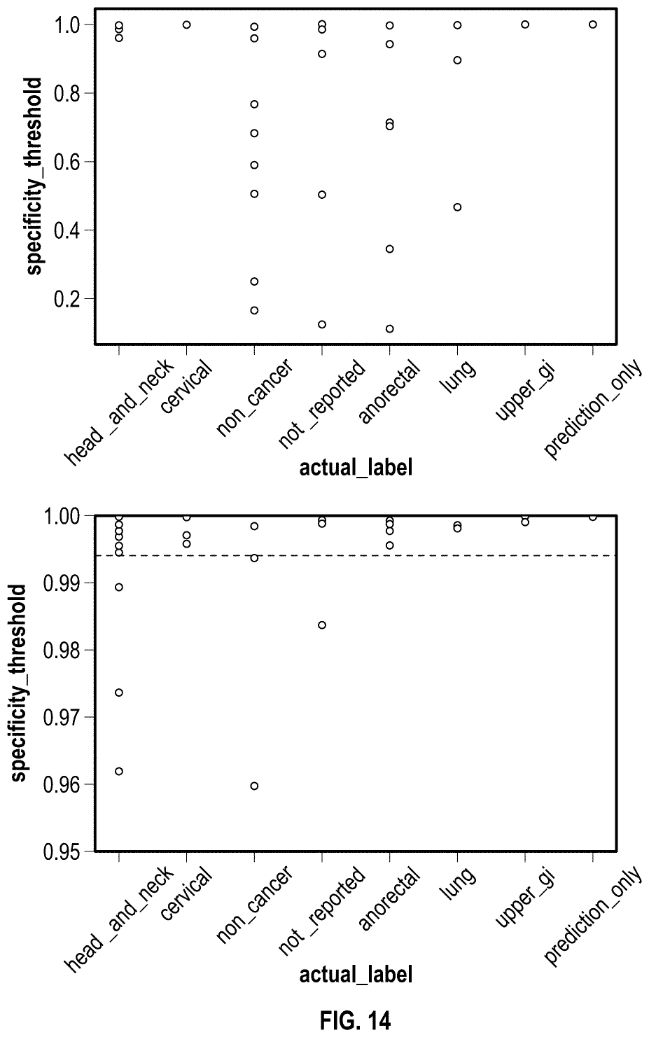

Turning now to FIG. 14, FIG. 14 provides graphs demonstrating that some currently undetected cancers are above certain specificity threshold cutoffs. Specifically, regarding the top graph of FIG. 14, a threshold of 5.8 is the 99.8th percentile of 4022 non-cancer training samples. In the bottom graph of FIG. 14, 134 samples (9 non-cancers, 125 cancers) are above the specificity threshold cutoff (dotted line), while 22 samples (8 non-cancers, 16 cancers) remain below the 0.994 specificity cutoff.

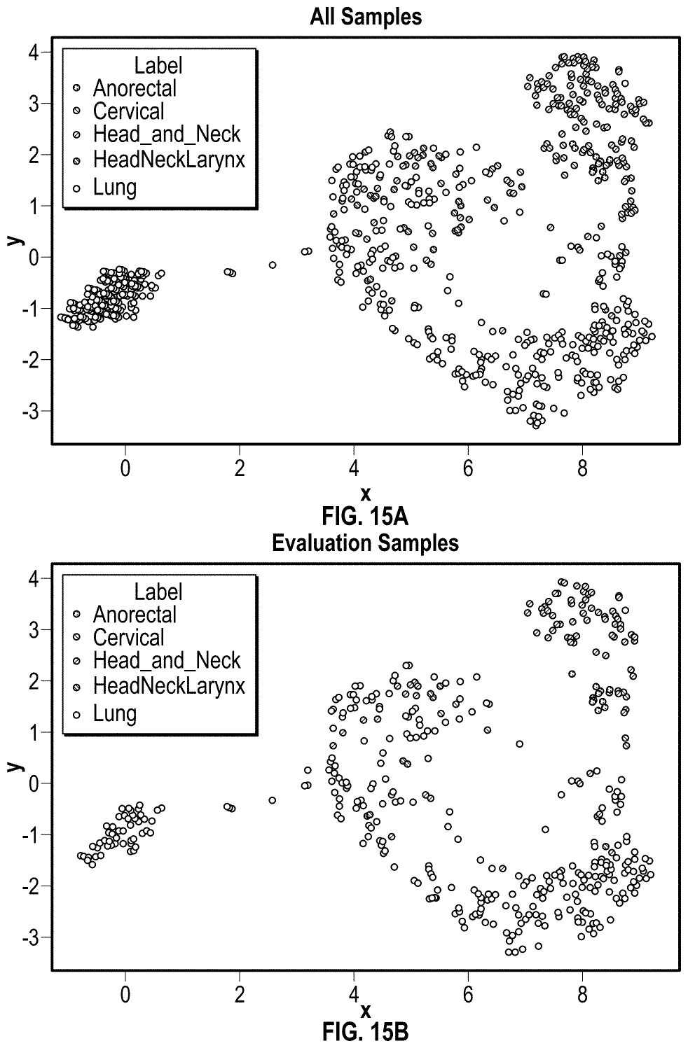

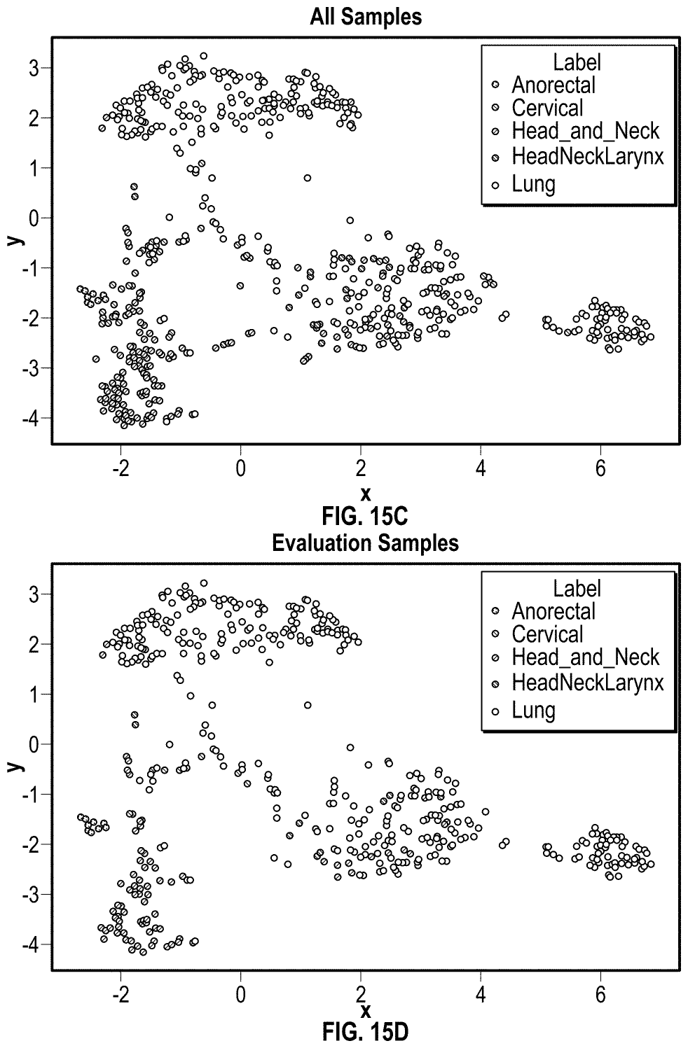

TOO classification confusion among head and neck cancers: Further observations from the dataset of this investigation show some TOO confusion with head and neck cancers. Specifically, a majority of detected anorectal samples were predicted as head and neck cancers (7/9). For example, a high proportion of head and neck samples were predicted as lung (7/54), which may be partially driven by larynx cancers. For instance, while larynx makes up about 12/111 of the head/neck cancers above a binary cutoff, 50% of lung misclassifications are larynx. FIGS. 15A-D show UMAP embeddings to illustrate that the observed TOO confusion can be seen at the feature level. For instance, FIG. 15A illustrates a UMAP embedding of features from a training set for all samples labeled anorectal, cervical, head and neck, head neck and larynx, and lung. FIG. 15B illustrates a UMAP embedding of features from a training set for evaluation samples also labeled anorectal, cervical, head and neck, head neck and larynx, and lung. As shown in both FIGS. 15A-B, clusters composed of a mixture of malignancies are present with little separation.

FIGS. 15C and 15D illustrate UMAP embeddings of certain selected features from a training set for all samples (FIG. 15C) and a training set for evaluation samples (FIG. 15D). Specifically, both figures use only the features where HPV positive type and HPV negative type is anorectal, cervical, head and neck, or lung.

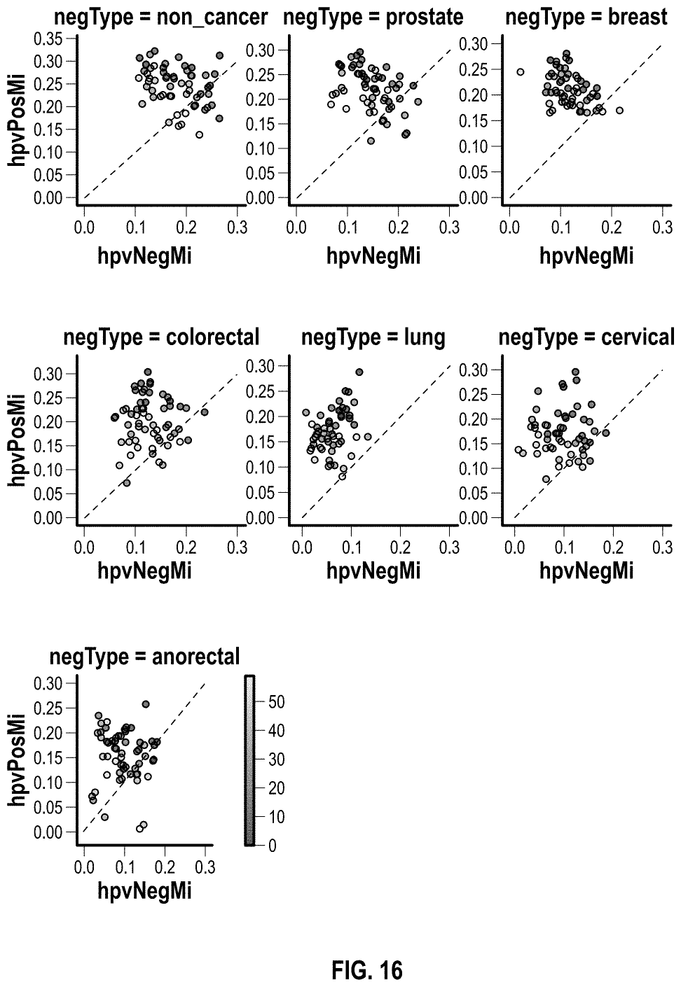

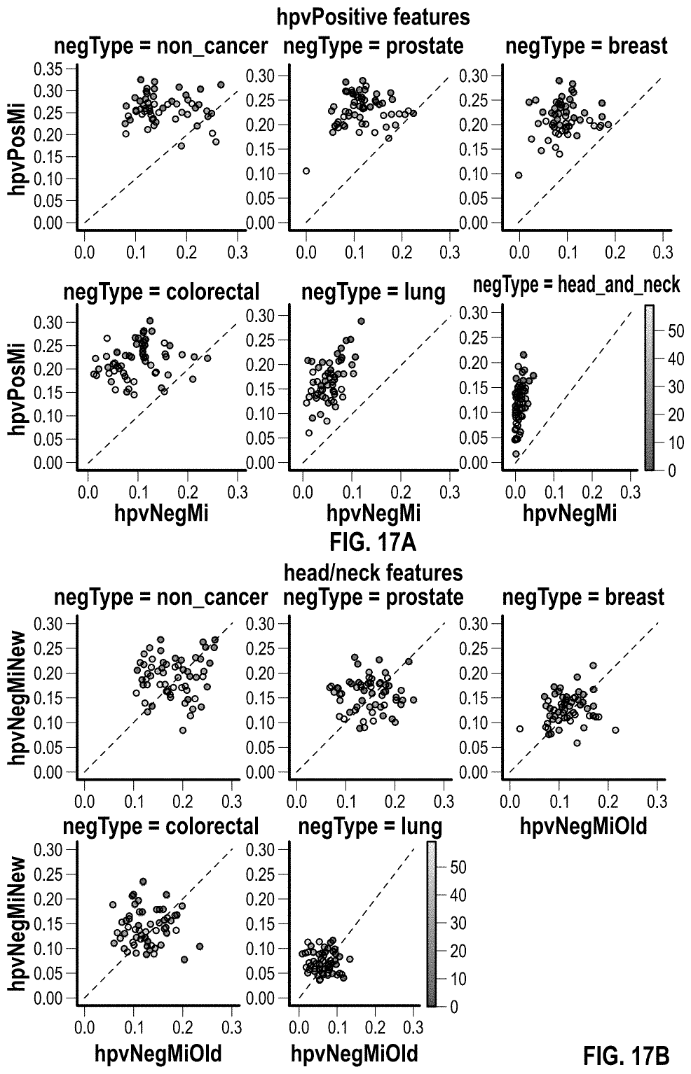

FIG. 16 illustrates various plots showing head and neck feature bias towards HPV positive patients. Various plots at FIGS. 17A-B show that separating HPV positive samples reduces the feature bias. For instance, the HPV positive samples can be separated by relabeling samples above a HPV cutoff as HPV positive (or otherwise having HPV presence). In some examples, such relabeling can be performed prior to feature selection. As shown at FIGS. 17A-B, head/neck features retain discrimination for head/neck samples, but now also have HPV status features (e.g., HPV positive features) that distinguish HPV-positive head/neck cancers.

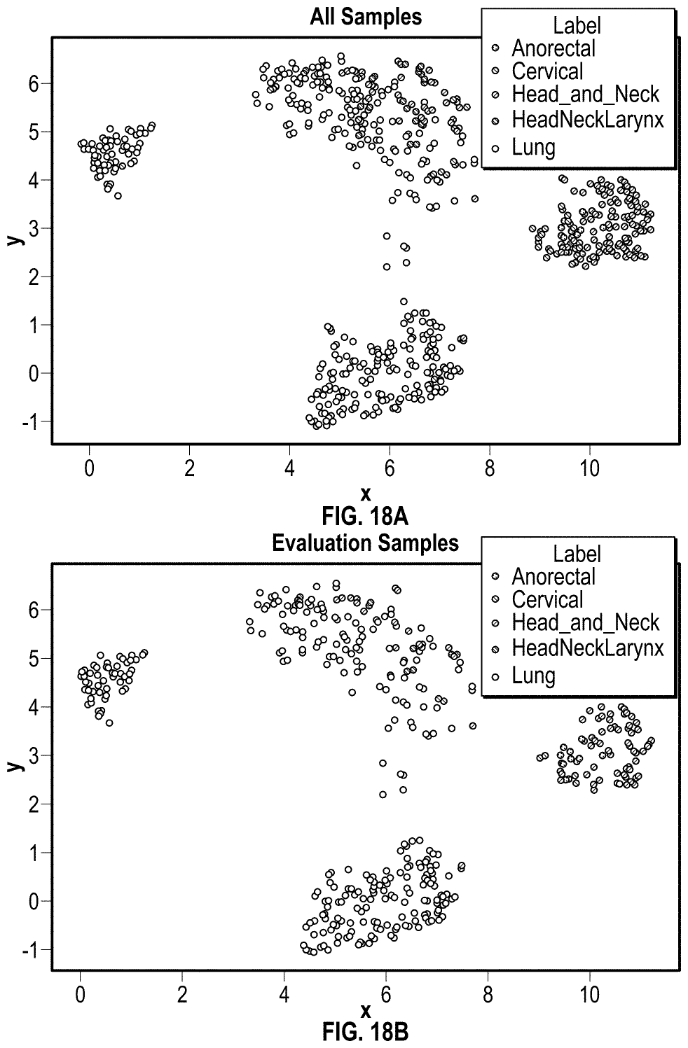

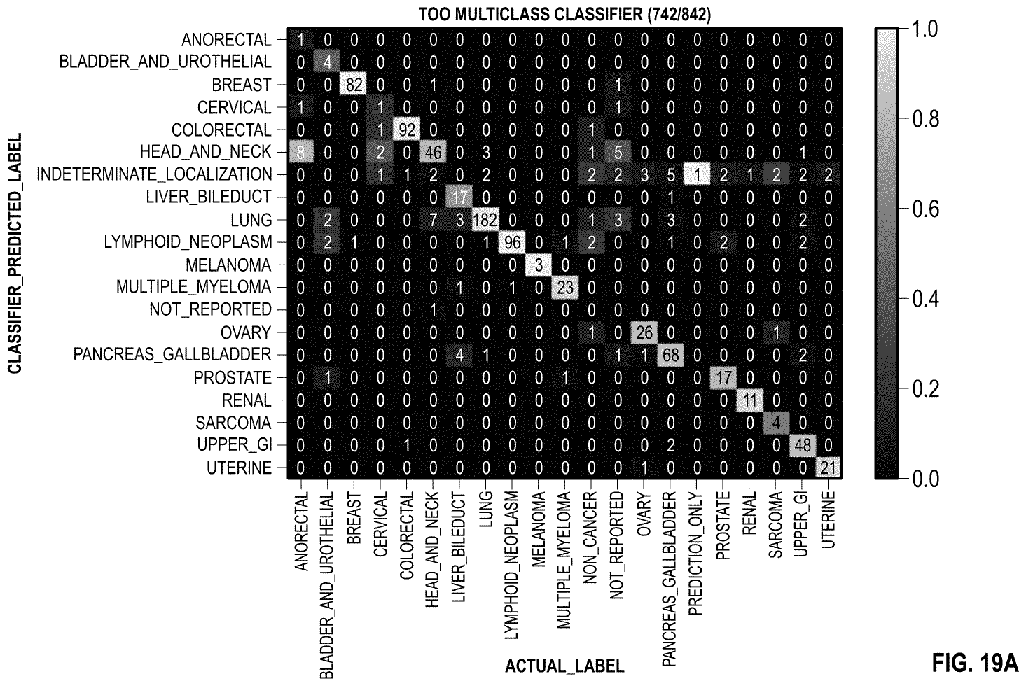

As shown at FIGS. 18A-B, HPV status features (i.e., HPV positive features) increase separation of HPV-associated cancers overall, compared to previous FIGS. 15A-D. FIGS. 18A-B illustrate UMAP embeddings of features from a train set for all samples and a train set for evaluation samples, respectively, after the reduction of head and neck feature bias, in accordance with various embodiments disclosed herein. Specifically, the UMAP embeddings at FIGS. 18A-B use only the features where HPV positive type and HPV negative type is anorectal, cervical, head and neck, or lung.

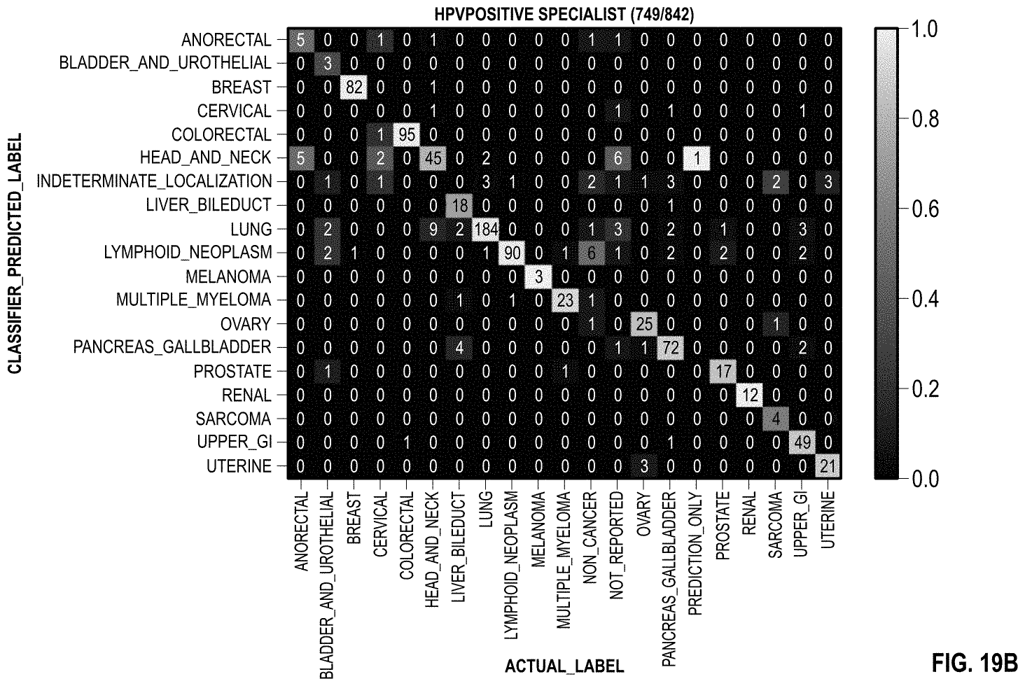

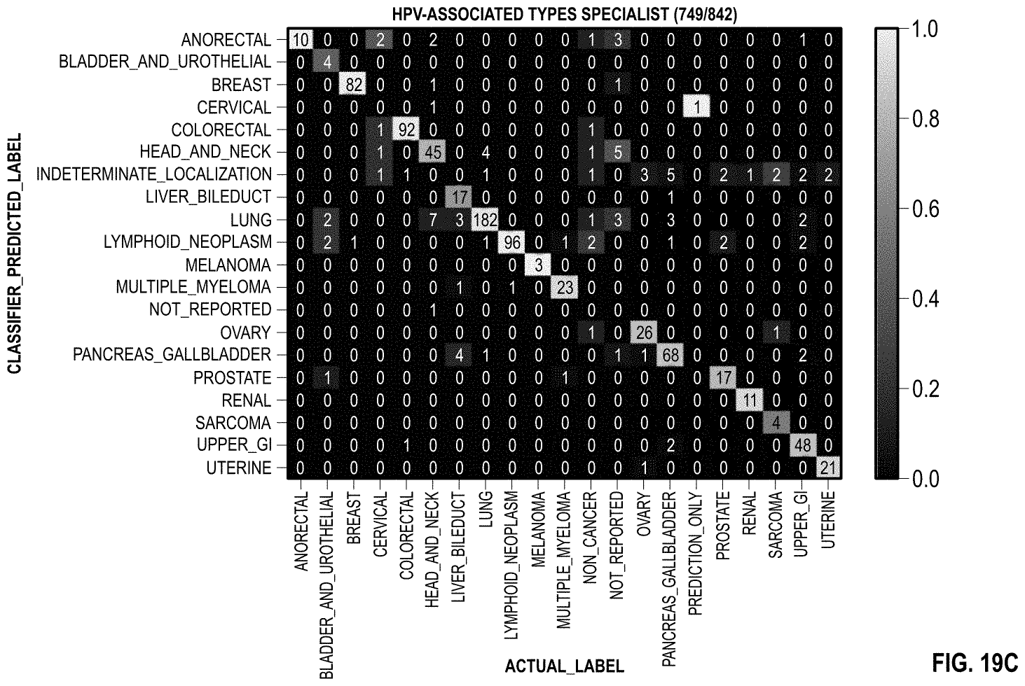

Classification performance: FIG. 19A illustrates a confusion matrix showing classification results of a TOO multiclass classifier that correctly predicted 742 of 842 samples. FIG. 19B demonstrates that classification using HPV status features can improve accuracy of classification, most notably within HPV-positive cancers. Specifically, FIG. 19B illustrates a confusion matrix showing classification results of an HPV-based multiclass classifier that correctly predicted 749 of the 842 samples. The HPV-based multiclass classifier was trained with anorectal, cervical, and head and neck cancer samples with HPV status (e.g., HPV positive) as an inner cross-validation prediction. At test time, any sample predicted as HPV-positive can be predicted with the HPV-based multiclass classifier.

FIG. 19C further demonstrates that applying a HPV-based multiclass classifier to the same featurization as that of the TOO multiclass classifier of FIG. 19A achieves better results than FIG. 19A alone (e.g., 742/842 for FIG. 19A vs. 749/842 for FIG. 19C). For instance, FIG. 19C illustrates a confusion matrix showing classification results of an HPV-based multiclass classifier trained with anorectal, cervical, and head and neck cancer samples passing a 95% specificity cutoff. At test time, any sample predicted as one of the three classes can be predicted with the HPV-based multiclass classifier. In some embodiments, the HPV-based multiclass classifier can be a classifier that is trained in accordance with any of the methods described herein. Such classifiers can be based on a logistic regression algorithm, a neural network algorithm, a support vector machine algorithm, or a decision tree algorithm that has been trained on a training cohort of subjects that includes subjects that have the cancer condition and/or subjects that do not have the cancer condition

### Example 4—HPV-Based Classification

Introduction: A noninvasive cell-free DNA (cfDNA)-based blood test designed to detect any cancer at pre-metastatic stages (stages I-III) could decrease cancer mortality. For such a multi-cancer test to be effective at population scale, it should: (i) Detect clinically significant cancers in an elevated risk population (e.g., older than 50 years) with a fixed and low false-positive rate (i.e., very high specificity, e.g., [>99%]) to limit overdiagnosis and unnecessary diagnostic workups; (ii) identify a specific tissue of origin (TOO) to direct appropriate diagnostic work-up for detected cancers; (iii) Be validated by prospective, multicenter, longitudinal, population-scale studies, with a large number of control individuals.

As previously described, the Circulating Cell-free Genome Atlas study (CCGA; NCT02889978) is a prospective, multi-center, case-control, observational study with longitudinal follow-up to support development of a plasma cfDNA-based multi-cancer early detection test. In the CCGA substudy 2, classifiers trained on methylation states in targeted genomic regions were used to detect cancer and predict TOO using cfDNA, achieving 99.3% specificity and 55% sensitivity. TOO was predicted in 96% of cases with a cancer-like signal; of these, the prediction was accurate in 93% of cases.

Some systematic misclassifications of head and neck (H&N) cancer with other cancers suggest biological complexity. High-risk human papillomavirus (HPV) infections have been implicated in the etiology of cervical cancer and other anogenital cancers, as well as cancers of the upper aerodigestive tract. Similarly, TOO misclassifications in the CCGA substudy 2 occurred between tissues commonly affected by HPV-associated cancers—anus, cervix, and clinically confirmed HPV-positive H&N (head and neck). Additionally, the TOO for cancers of the vulva and penis was predicted as H&N. TOO misclassification was also observed between H&N and lung cancers; this could be driven by commonalities in cancer type and site (squamous cell carcinomas of the upper airways and larynx), and risk factor (exposure to carcinogens from smoking).

This post-hoc analysis of a subgroup of participants from CCGA was aimed to (a) explore the hypothesis that TOO misclassifications among HPV-associated cancers are driven by epigenetic similarity due to underlying HPV infections, and (b) improve the accuracy of TOO predictions for HPV-associated cancer types.

Methods

Detection of HPV DNA Fragments in Plasma cfDNA Samples: Sample collection, accessioning, storage, and processing were conducted as previously described. Additionally, the hybridization capture panel contained probes targeting the HPV 16 and HPV 18 genomes. Probes were designed to tile the entire genomes and target both methylated and unmethylated copies of each sequence (assuming uniform methylation status). HPV 16 and HPV 18 are high-risk HPV types most commonly associated with cancer types such as cervical, anogenital, and H&N cancers. Plasma cfDNA samples of all participants were assessed for presence of HPV DNA fragments by counting the number of unique fragments mapping to the HPV 16 and HPV 18 genomes. For a subset of participants (n=57), HPV status was established based on pathology reports.

Cancer Status Classification Using HPV DNA Fragments in Plasma cfDNA Samples Versus Methylation Features: Classification of cancer status using HPV DNA fragments in plasma cfDNA samples was performed using a cross-validated cutoff on the number of unique cfDNA fragments aligned to HPV 16 and HPV 18 targets in a sample. Classification of cancer status and TOO was conducted as previously described using a methylation-based classifier.

Visualization of Methylation Features Among Misclassified Tissues: To create an informative embedding, this investigation first subsets to methylation features that were selected by the classifier as discriminatory in pairwise comparisons among HPV-associated cancer types and pairwise comparisons to lung cancers. Selected features were used to create a UMAP embedding of participants with the cancer types of interest, subset to cancers used to train the TOO classifier.

Development of a Specialist Classifier for TOO Prediction of HPV-Associated Cancers: As an addition to the original methylation-based TOO classifier, a three class logistic regression classifier was trained using the same methylation features but restricted to cervical, anal, and H&N cancers. This specialized classifier was applied to produce new predictions for samples predicted as any of the three cancers by the methylation-based TOO classifier.

Results

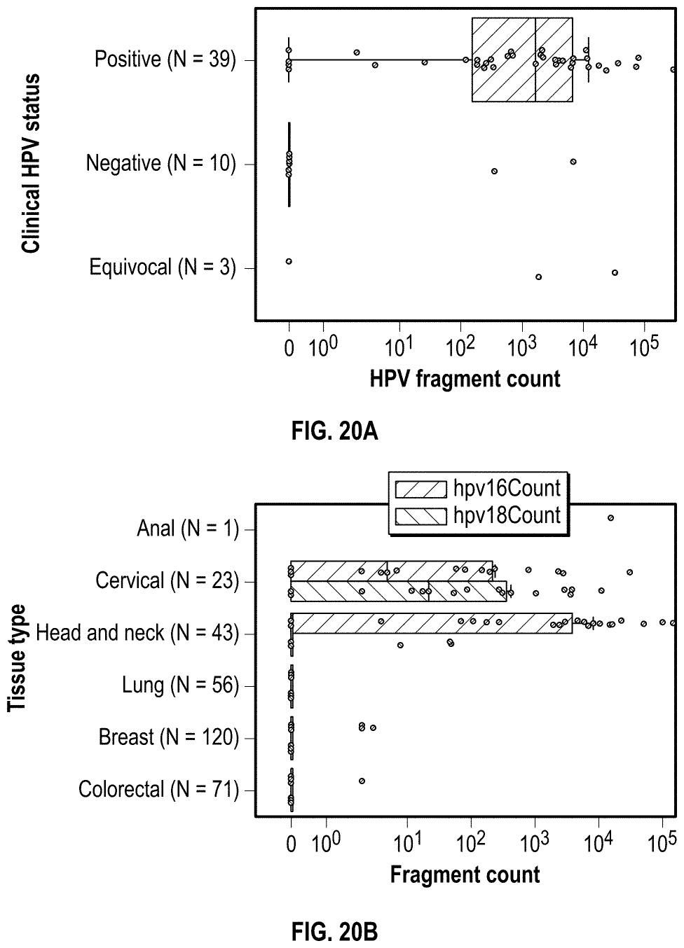

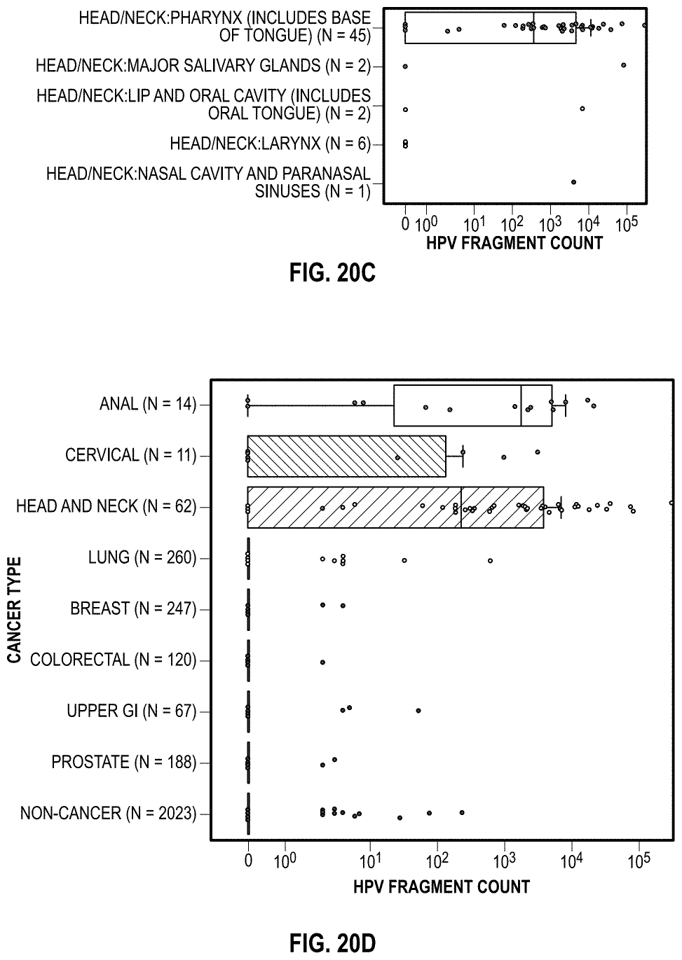

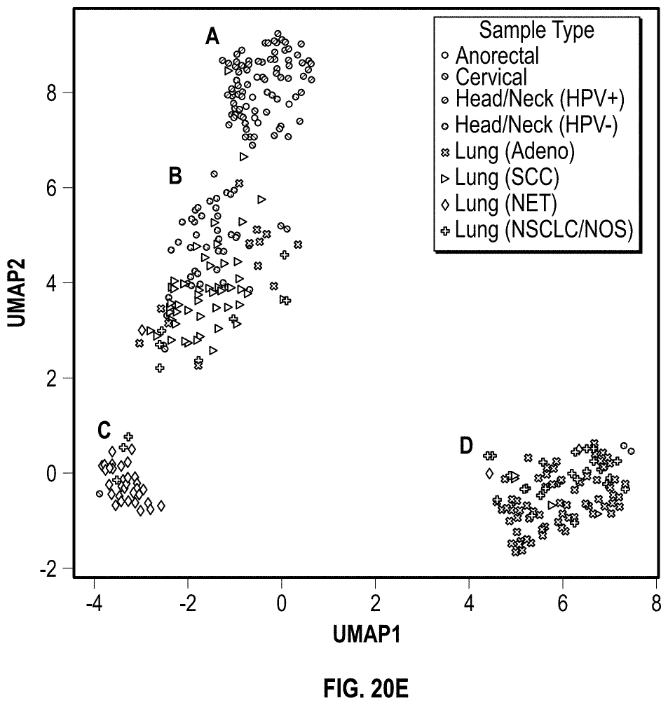

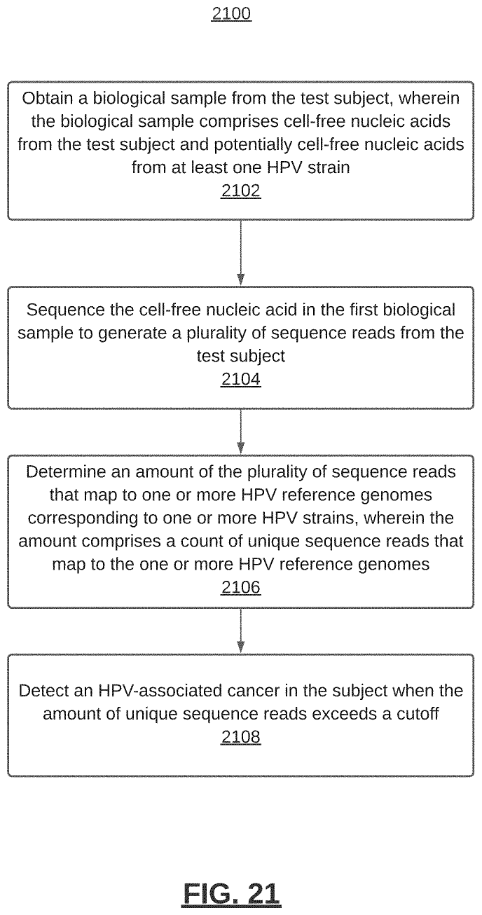

Detection of HPV DNA Fragments in Plasma cfDNA Samples: Of the overall population (N=3553; cancer, n=1530; non-cancer, n=2023), 72 had an HPV-associated cancer and 3481 did not have an HPV-associated cancer. HPV DNA fragment counts (HPV 16+HPV 18 DNA fragment counts) in plasma cfDNA samples were mostly concordant with clinical diagnosis of HPV status, when available. For instance, FIG. 20A illustrates a bar chart showing HPV DNA fragment counts by clinically diagnosed HPV status, subset to high-signal plasma cfDNA samples and detected as having cancer. As shown at FIG. 20A, presence of HPV DNA fragments in plasma cfDNA samples was more likely in participants with clinically confirmed HPV-positive status versus those with HPV-negative status.

Of the tumor biopsies with HPV DNA fragments, HPV 18 DNA fragments were most frequently observed in tumor biopsies of cervical cancer (84%); this aligned with reports of higher rates of HPV 18 infection in cervical cancer versus anal and H&N cancer in literature. For instance, FIG. 20B illustrates a bar chart showing HPV 16 versus HPV 18 DNA fragment counts in tumor biopsies by tissue type, and subset to tumor biopsy samples due to low number of plasma cfDNA samples from participants with cervical cancer. HPV 18 DNA fragments were most frequently observed in participants with cervical cancer. 84% (16/19) of tumor biopsies with non-zero HPV 18 DNA fragment counts are cervical cancer.

Among participants with H&N cancer, HPV DNA fragments were mainly detected in participants with tumors in the oropharyngeal region as opposed to tumors in the larynx and oral cavity; this aligned with reports of HPV-associated H&N cancers being more frequently observed in the oropharynx. For instance, FIG. 20C illustrates a bar chart showing HPV DNA fragment counts in head and neck cancer participants by tumor location, subset to high-signal plasma cfDNA samples and detected as having cancer. HPV DNA fragment counts were higher in participants with tumors in the oropharyngeal region versus those with tumors in the larynx and oral cavity.

Presence of HPV DNA fragments in plasma cfDNA samples were observed to be a highly specific indicator of HPV-associated cancer. In particular, HPV DNA fragments were detected in the plasma cfDNA samples of only 1.1% (40/3481) of participants with no reported HPV-associated cancer. For instance, FIG. 20D illustrates a bar chart showing HPV DNA fragment counts in plasma cfDNA samples by cancer type, and showing all cfDNA samples. HPV DNA fragment counts in cfDNA samples were highest in participants with HPV-associated cancers such as H&N, cervical, and anorectal cancer.

Cancer Status Classification Using HPV DNA Fragments in Plasma cfDNA Samples Versus Methylation Features: A cross-validated cutoff on the number of HPV DNA fragments in a plasma cfDNA sample (5.4±1.2, across 6 folds) demonstrated high sensitivity for HPV-associated cancers at 99.8% specificity, achieving performance similar to the original methylation-based classifier for those cancer types (Table 3). The high specificity of HPV DNA fragments in plasma cfDNA samples for HPV-associated cancers, despite the prevalence of transient HPV infections in the US, was consistent with the lack of HPV viremia reported in literature.

Table 3. Comparison of Specificity and Sensitivity for Cross-Validated HPV DNA Fragment Cutoff and the Methylation-Based Classifier.

TABLE 3

| | HPV DNA | Methylation- |
|---|---|---|
| | Fragment Cutoff | Based Classifier |
| Specificity | 99.8% (2018/2023) | 99.6% (2015/2023) |

Sensitivity

| | | |
|---|---|---|
| Non-HPV-associated | 0.8% (11/1458) | 53.8% (785/1458) |
| cancers | | |
| HPV-associated | 72.2% (52/72) | 79.1% (57/72) |
| cancers | | |
| Anus | 78.6% (11/14) | 71.4% (10/14) |
| Cervix | 36.4% (4/11) | 45.4% (5/11) |
| HPV-positive H&N | 81.1% (30/37) | 97.3% (36/37) |
| Vulva | 66.7% (6/9) | 55.6% (5/9) |
| Penis | 100% (1/1) | 100% (1/1) |

Visualization of Methylation Features Among Misclassified Tissues: In the UMAP embedding at FIG. 20E, four distinct groups of participants were observed generally separated by lung cancer subtype and HPV signal (defined as presence or absence of HPV DNA fragments in plasma). Some notable exceptions to the participant clustering included: (i) HPV signal negative neuroendocrine cervical cancer (n=1) clustered with lung neuroendocrine tumor (NET; n=39) (cluster C); (ii) HPV signal negative cervical adenocarcinoma (n=1) and HPV signal negative salivary gland cancers of the H&N (n=2) clustered with lung adenocarcinoma (n=79) and non-small cell lung cancer (NSCLC; n=26) (cluster D).

There were 6 HPV signal negative H&N cancer participants clustered with the HPV-associated cancers group (cluster A). Of these, 3 participants had sequenced tumor biopsies, all of which had non-zero HPV DNA fragments, indicating that the selected methylation features are informative for HPV signal in absence of observed HPV DNA fragments in plasma cfDNA samples. Table 4 illustrates that visualization of methylation features among misclassified tissues showed four distinct groups of participants generally separated by lung cancer subtype and HPV signal. It is noted that Table 4 is subset to cancers used to train the TOO classifier, and representations H&N refers to head and neck, HPV to human papillomavirus, NET to neuroendocrine tumor, NOS to not otherwise specified, NSCLC to non-small cell lung cancer, and SCC to squamous cell carcinoma.

TABLE 4

| | Cluster | | | | |
|---|---|---|---|---|---|
| | | B: | | | |
| | | Predominan | | | |
| | | tly Non- | | D: | |
| | | HPV- | C: | Predominan- | |
| | | Associated | Predominan- | tly Non-HPV- | |
| | A: HPV- | Squamous | tly | Associated | |
| | Associated | Cell | Neuroendoc | Adenocarcin | |
| Cancer Type | Cancers | Carcinomas | rine Tumors | omas | Total |
| Anal | 7 | 1 | 0 | 0 | 8 |
| Cervical | 20 | 6 | 1 | 1 | 28 |
| H&N (HPV signal +) | 57 | 1 | 0 | 0 | 58 |
| H&N (HPV signal −) | 6 | 39 | 0 | 2 | 47 |
| Lung (Adenocarcinoma) | 0 | 12 | 0 | 79 | 91 |
| Lung (NET) | 0 | 1 | 39 | 2 | 42 |
| Lung (NSCLC/NOS) | 0 | 7 | 3 | 26 | 36 |
| Lung (SCC) | 2 | 57 | 0 | 5 | 64 |

Development of a Specialist Classifier for TOO Prediction of HPV-Associated Cancers: The development of a specialist classifier was motivated by the observation that despite the HPV-associated cancers forming a single cluster separate from HPV-signal-negative samples, the HPV-associated cluster appeared to show some substructure and separation of H&N cancers from anal and cervical cancers. Applying the specialist classifier resulted in an increase in TOO prediction accuracy of anal cancers (Table 5).

Table 5. Comparison of TOO Prediction Accuracy for the HPV Specialist Classifier and the Methylation-Based Classifier

TABLE 5

| | Prediction Accuracy | |
|---|---|---|
| | | Methylation-Based Classifier + |
| | | Methylation-Based |
| | Methylation-Based | HPV Specialist |
| Cancer Type | Classifier | Classifier |
| Non-HPV associated | 89.8% (705/785) | 89.8% (705/785) |
| cancers | | |
| Anal | 10% (1/10) | 100% (10/10) |
| Cervical | 20% (1/5) | 0% (0/5) |
| HPV + head/neck | 97.2% (35/36) | 94.4% (34/36) |

### Example Methods

FIGS. 21-25 illustrate various methods for detecting HPV-based cancers, in accordance with various embodiments described herein. It is noted that in any of the methods of FIGS. 21-25, some operations can be combined with any of the operations or embodiments disclosed elsewhere herein, the order of some operations can be changed, and some operations can be omitted.

FIG. 21 is a flow diagram illustrating method 2100 of screening for detecting an HPV-associated cancer in a subject, in accordance with various embodiments. The method 2100 can include, at block 2102, obtaining a biological sample from the test subject. The biological sample can include cell-free nucleic acids from the test subject and potentially cell-free nucleic acids from at least one HPV strain. In some examples, the one or more HPV strains includes HPV 16 and/or HPV 18. In some examples, the one or more HPV strains include one or more of HPV 16, 18, 31, 33, 35, 39, 45, 51, 52, 56, 58, 59, 66 and 68.

The method 2100 can include, at block 2104, sequencing the cell-free nucleic acid in the first biological sample to generate a plurality of sequence reads from the test subject. In some examples, the sequencing includes whole genome sequencing, targeted sequencing, or whole genome bisulfite sequencing, as described elsewhere herein.

The method 2100 can include, at block 2106, determining an amount of the plurality of sequence reads that map to one or more HPV reference genomes corresponding to one or more HPV strains. The amount can include a count of unique sequence reads that map to the one or more HPV reference genomes. For instance, the amount of unique sequence reads can include a total count of unique sequence reads that map to one or more HPV reference genomes corresponding to the one or more HPV strains.

The method 2100 can include, at block 2108, detecting an HPV-associated cancer in the subject when the amount of unique sequence reads exceeds a cutoff. In some cases, the HPV-associated cancer is cervical, anogenital, and/or head and neck cancer. In some examples, the cutoff is 5 unique sequence reads, more than 10 unique sequence reads, or more than 20 unique sequence reads. Further, in some examples, the cutoff is a cross-validated HPV DNA fragment count cutoff associated with a target specificity for detecting HPV-associated cancers. Merely by way of example, the target specificity can be within the range of 99.0-99.9%.

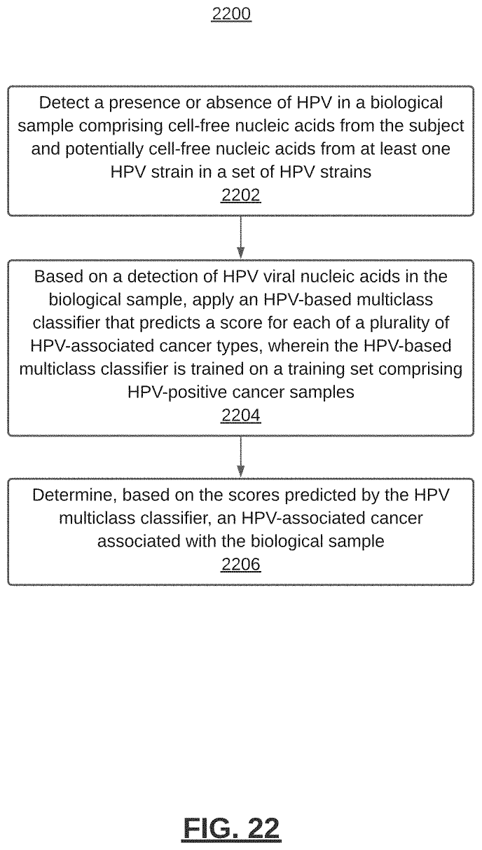

Turning now to FIG. 22, FIG. 22 is a flow diagram illustrating method 2200 of screening for presence of an HPV-associated cancer in a subject, in accordance with various embodiments. The method 2200 can include, at block 2202, detecting a presence or absence of HPV in a biological sample comprising cell-free nucleic acids from the subject and potentially cell-free nucleic acids from at least one HPV strain in a set of HPV strains. In some examples, detecting the presence or absence of HPV viral nucleic acids in the biological sample includes determining an amount of HPV fragments in the biological sample that are derived from the potentially cell-free nucleic acid from the at least one HPV strain in the set of HPV strains, comparing the amount of HPV fragments to a cutoff, and detecting HPV presence in the biological sample when the amount exceeds the cutoff. In some cases, determining the amount of HPV fragments involves sequencing the cell-free nucleic acids and potentially cell-free nucleic acids from one or more HPV strains to obtain a plurality of sequence reads, and determining the amount of HPV fragments based on a total count of the plurality of sequence reads that map to one or more HPV reference genomes corresponding to the one or more HPV strains. The sequencing can be performed by whole genome sequencing, targeted sequencing, or whole genome bisulfite sequencing. In some examples, sequencing is performed by targeted sequencing with a hybridization capture panel containing probes targeting HPV reference genomes corresponding to the set of HPV strains. Such probes can tile the targeted HPV reference genomes.

In some cases, the cutoff is a count of at least 6 unique HPV fragments, where each unique HPV fragment maps to an HPV reference genome corresponding to at least one HPV strain in the set of HPV strains. The set of HPV strains can include at least one of HPV 16 or HPV 18. In some cases, the set of HPV strains includes one or more of HPV 16, 18, 31, 33, 35, 39, 45, 51, 52, 56, 58, 59, 66 and 68.

The method 2200 can include, at block 2204, based on a detection of HPV viral nucleic acids in the biological sample, applying an HPV-based multiclass classifier that predicts a score for each of a plurality of HPV-associated cancer types, wherein the HPV-based multiclass classifier is trained on a training set comprising HPV-positive cancer samples. The HPV-based multiclass classifier can predict the scores based on features derived from sequencing the potentially cell-free nucleic acids from the at least one HPV strain in a set of HPV strains in the biological sample. The features can include one or more of methylation-derived features, a total count of HPV fragments, and a binarized count of HPV fragments. In some examples, the methylation-derived features are features that discriminate pairwise comparisons among HPV-associated cancer types and other cancer types, such as lung cancers.

In some examples, the plurality of HPV-associated cancer types include cervical, anogenital, and head and neck cancers. The HPV-based multiclass classifier can include a multinomial logistic regression classifier. In some cases, training of the HPV-based multiclass classifier is restricted to the HPV-positive cancer samples, whereby the HPV-positive cancer samples are associated with at least one of cervical, anorectal, and head and neck cancers.

The method 2200 can include, at block 2206, based on the scores predicted by the HPV multiclass classifier, an HPV-associated cancer associated with the biological sample. Further, in some examples, the method 2200 can include, based on a detection of HPV absence from the biological sample: forgoing applying the HPV-based multiclass classifier, or determining an absence of HPV-associated cancer from the biological sample.

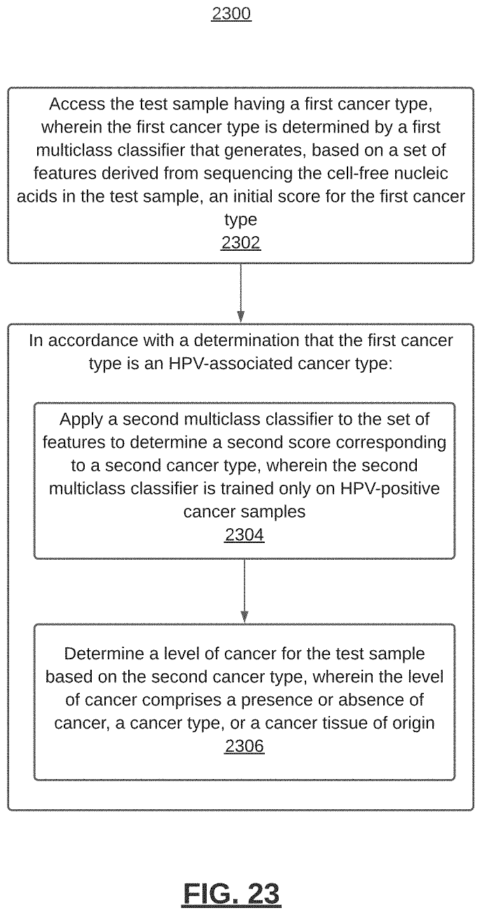

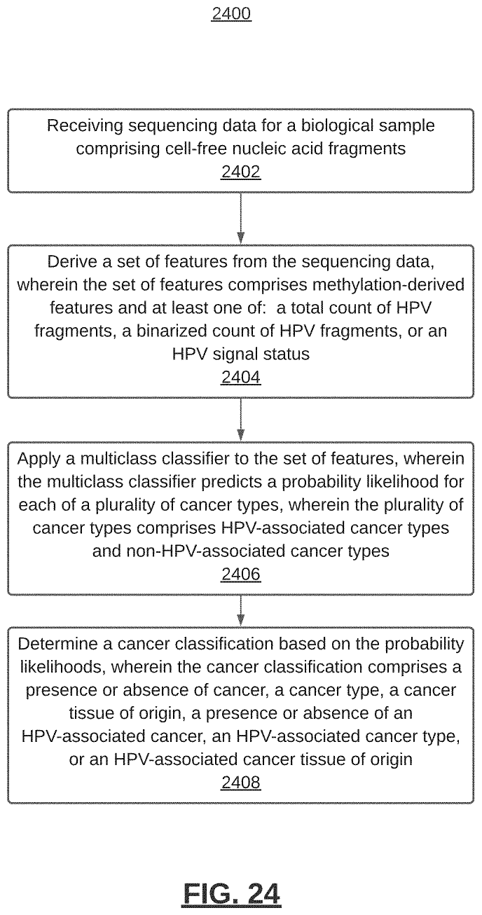

Turning now to FIG. 23, FIG. 23 is a flow diagram illustrating method 2300 of predicting a presence or absence of cancer in a test sample containing cell-free nucleic acids, such as cell-free nucleic acids from a test subject and potentially cell-free nucleic acids from at least one HPV strain, in accordance with various embodiments. The method 2300 can include, at block 2302, accessing the test sample having a first cancer type. The first cancer type can be determined by a first multiclass classifier that generates, based on a set of features derived from sequencing the cell-free nucleic acids in the test sample, an initial score for the first cancer type. The sequencing can be performed by whole genome sequencing, targeted sequencing, or whole genome bisulfite sequencing. In some examples, the sequencing includes a targeted pulldown of HPV 16 and HPV 18 nucleic acid sequences in the cell-free nucleic acid in the test sample.

The method 2300 can include, at block 2304, in accordance with a determination that the first cancer type is an HPV-associated cancer type: applying a second multiclass classifier to the set of features to determine a second score corresponding to a second cancer type, whereby the second multiclass classifier is trained only on HPV-positive cancer samples. Merely by way of example, the HPV-associated cancer type can be cervical, anogenital, or head and neck cancer.

In some examples, the first multiclass classifier can include a plurality of classes corresponding to a plurality of HPV-associated cancer types and non-HPV-associated cancer types. In some examples, the second multiclass classifier can include at least three classes corresponding to three HPV-associated cancer types, such as cervical, anogenital, and head and neck cancers. The first multiclass classifier can be trained using a set of training features derived from a plurality of HPV-associated cancer type samples and non-HPV-associated cancer type samples, the set of training features including methylation-derived features, and the second multiclass classifier can be trained using a restricted set of training features from the set of training features, the restricted set of training features being restricted to features derived from the plurality of HPV-associated cancer type samples.

In some examples, features in the set of features include one or more methylation-derived features, a total count of HPV fragments, a binarized count of HPV fragments, and/or an HPV signal status. For instance, the total count of HPV fragments or the binarized count of HPV fragments can include a quantified count of unique sequence reads mapping to HPV 16 and/or HPV 18 reference genomes. The HPV signal status can include an HPV-positive signal status defined by a presence of HPV cell-free nucleic acid fragments or an HPV-negative signal status defined by an absence of HPV cell-free nucleic acid fragments (e.g., with respect to a cutoff or threshold count of fragments detected). For instance, in some examples, the HPV cell-free nucleic acid fragments are confirmed when a quantification of unique sequence reads mapping to HPV 16 and HPV 18 reference genomes is greater than a threshold. Merely by way of example, the threshold can be approximately 6 unique sequence reads mapping to HPV 16 and HPV 18 reference genomes, or any threshold range of fragments such as a threshold between 5-7 unique sequence reads, 4-8 unique sequence reads, and/or 3-9 unique sequence reads.

The total count of HPV fragments or the binarized count of HPV fragments can include a quantified count of unique sequence reads mapping to one or more HPV reference genomes. The HPV signal status can include an HPV-positive signal status defined by a presence of HPV cell-free nucleic acid fragments or an HPV-negative signal status defined by an absence of HPV cell-free nucleic acid fragments, whereby presence of the HPV cell-free nucleic acid fragments is confirmed when a quantification of unique sequence reads mapping to one or more HPV reference genomes is greater than a threshold (e.g., a threshold of 6 unique sequence reads mapping to one or more HPV reference genomes). Such HPV reference genomes can be associated with one or more strains of HPV 16, 18, 31, 33, 35, 39, 45, 51, 52, 56, 58, 59, 66 and 68.

The method 2300 can include, at block 2306, determining a level of cancer for the test sample based on the second cancer type. The level of cancer can include a presence or absence of cancer, a cancer type, or a cancer tissue of origin. In some examples, the method 2300 can include, in accordance with a determination that the first cancer type is not an HPV-associated cancer type, forgoing applying the second multiclass classifier to the set of features, and determining a level of cancer for the test sample based on the first cancer type, wherein the level of cancer is a presence or absence of cancer, a cancer type, or a cancer tissue of origin.

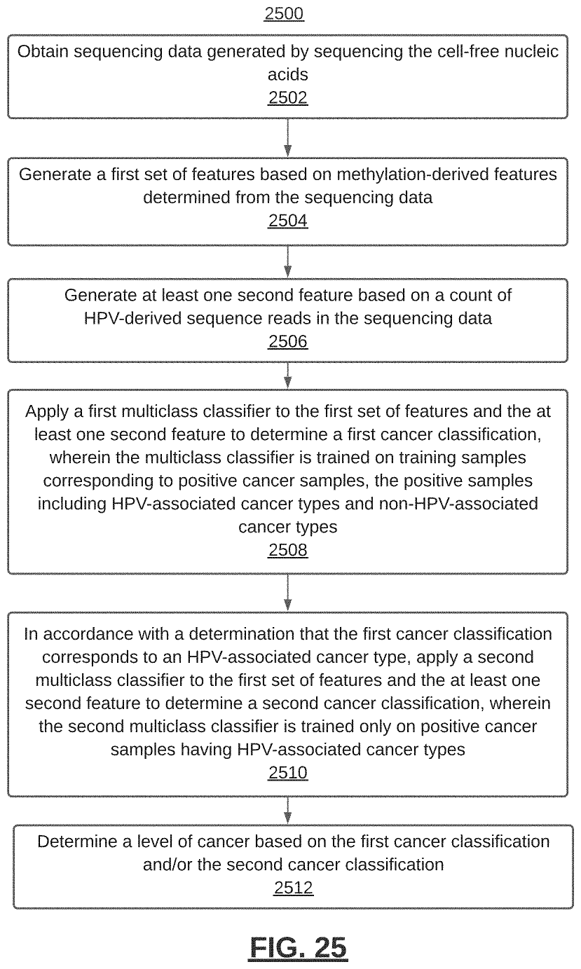

Turning now to FIG. 24, FIG. 24 is a flow diagram illustrating method 2400 of detecting and classifying cancer, in accordance with various embodiments. The method 2400 can include, at block 2402, receiving sequencing data for a biological sample comprising cell-free nucleic acid fragments. The method 2400 can include, at block 2404, deriving a set of features from the sequencing data, whereby the set of features includes methylation-derived features and at least one of a total count of HPV fragments, a binarized count of HPV fragments, or an HPV signal status. Further, the method 2400 can include, at block 2406, applying a multiclass classifier to the set of features, wherein the multiclass classifier predicts a probability likelihood for each of a plurality of cancer types, wherein the plurality of cancer types includes HPV-associated cancer types and non-HPV-associated cancer types. The method 2400 can include, at block 2408, determining a cancer classification based on the probability likelihoods, wherein the cancer classification comprises a presence or absence of cancer, a cancer type, a cancer tissue of origin, a presence or absence of an HPV-associated cancer, an HPV-associated cancer type, or an HPV-associated cancer tissue of origin.

Various operations and features of the method 2400 can be combined with any of the embodiments, examples, and aspects described elsewhere herein. Additionally, in some cases, a threshold for calling a tissue of origin or cancer signal origin (e.g., a cancer-positive determination) for a sample can be dynamically and/or automatically set based on an HPV-related feature derived from sequencing that sample. For instance, if a cutoff number of HPV fragments is detected in the sample (e.g., at least 6 fragments), then in some embodiments, the threshold/score for determining whether a sample is cancer-positive can be lower than for samples where no HPV fragment is detected or for samples where the cutoff number of HPV fragments has not been met. The dynamic thresholding can apply to binary cancer classifiers, where the threshold is dynamic for calling cancer vs. non-cancer for a sample. The dynamic thresholding can apply to multiclass cancer classifiers, where the threshold is dynamic for calling certain types of cancers, such as for calling HPV-associated cancers.

FIG. 25 is a flow diagram illustrating method 2500 of detecting a level of cancer in a test sample comprising cell-free nucleic acids from a test subject and potentially cell-free nucleic acids from a HPV strain, in accordance with various embodiments. The method 2500 can include, at block 2502, obtaining sequencing data generated by sequencing the cell-free nucleic acids. The method 2500 can include, at block 2504, generating a first set of features based on methylation-derived features determined from the sequencing data. The method 2500 can include, at block 2506, generating at least one second feature based on a count of HPV-derived sequence reads in the sequencing data.

The method 2500 can include, at block 2508, applying a first multiclass classifier to the first set of features and the at least one second feature to determine a first cancer classification, wherein the multiclass classifier is trained on training samples corresponding to positive cancer samples, the positive samples including HPV-associated cancer types and non-HPV-associated cancer types. The method 2500 can include, at block 2510, in accordance with a determination that the first cancer classification corresponds to an HPV-associated cancer type: applying a second multiclass classifier to the first set of features and the at least one second feature to determine a second cancer classification, wherein the second multiclass classifier is trained only on positive cancer samples having HPV-associated cancer types. Further, the method 2500 can include, at block 2512, determining a level of cancer based on the first cancer classification and/or the second cancer classification.

Various operations and features of the method 2500 can be combined with any of the embodiments, examples, and aspects described elsewhere herein.

### CONCLUSIONS

HPV infection can induce similar epigenetic changes across multiple tissue types; although this could cause TOO misclassification, it indicates that the methylation-based classifier has learned to classify plasma cfDNA samples using epigenetic markers that reflect underlying biological signals and pathological processes. The presence of HPV DNA fragments in plasma cfDNA samples is a highly specific indicator of HPV-associated cancer. Understanding the underlying cause of TOO misclassification can inform changes to classification architecture that could improve overall TOO prediction accuracy, furthering the goal of guiding effective clinical follow-up after signal detection from a multi-cancer early detection test

### Additional Considerations

It is to be understood that the figures and descriptions of the present disclosure have been simplified to illustrate elements that are relevant for a clear understanding of the present disclosure, while eliminating, for the purpose of clarity, many other elements found in a typical system. Those of ordinary skill in the art may recognize that other elements and/or steps are desirable and/or required in implementing the present disclosure. However, because such elements and steps are well known in the art, and because they do not facilitate a better understanding of the present disclosure, a discussion of such elements and steps is not provided herein. The disclosure herein is directed to all such variations and modifications to such elements and methods known to those skilled in the art.

Some portions of the above description describe the embodiments in terms of algorithms and symbolic representations of operations on information. These algorithmic descriptions and representations are commonly used by those skilled in the data processing arts to convey the substance of their work effectively to others skilled in the art. These operations, while described functionally, computationally, or logically, are understood to be implemented by computer programs or equivalent electrical circuits, microcode, or the like. The described operations and their associated modules may be embodied in software, firmware, hardware, or any combinations thereof.

The methods of the invention may be accomplished using robotics controlled by computers. The methods may be embodied in computer-readable instructions for controlling robotic operations to cause them to execute the disclosed methods.

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment, thereby providing a framework for various possibilities of described embodiments to function together.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

In addition, use of the “a” or “an” are employed to describe elements and components of the embodiments herein. This is done merely for convenience and to give a general sense of the invention. This description should be read to include one or at least one and the singular also includes the plural unless it is obvious that it is meant otherwise.

While particular embodiments and applications have been illustrated and described, it is to be understood that the disclosed embodiments are not limited to the precise construction and components disclosed herein. Various modifications, changes and variations, which will be apparent to those skilled in the art, may be made in the arrangement, operation and details of the method and apparatus disclosed herein without departing from the spirit and scope defined in the appended claims.

What is claimed is:

1-11. (canceled)

12. A method of screening for presence of an HPV-associated cancer in a subject, comprising:
detecting a presence or absence of HPV in a biological sample comprising cell-free nucleic acids from the subject and potentially cell-free nucleic acids from at least one HPV strain in a set of HPV strains;
based on a detection of HPV viral nucleic acids in the biological sample, applying an HPV-based multiclass classifier that predicts a score for each of a plurality of HPV-associated cancer types, wherein the HPV-based multiclass classifier is trained on a training set comprising HPV-positive cancer samples; and
determining, based on the scores predicted by the HPV multiclass classifier, an HPV-associated cancer associated with the biological sample.

13. The method according to claim 12, wherein detecting the presence or absence of HPV viral nucleic acids in the biological sample comprises:
determining an amount of HPV fragments in the biological sample that are derived from the potentially cell-free nucleic acid from the at least one HPV strain in the set of HPV strains;
comparing the amount of HPV fragments to a cutoff; and
detecting HPV presence in the biological sample when the amount exceeds the cutoff.

14. The method according to claim 12, wherein determining the amount of HPV fragments comprises:
sequencing the cell-free nucleic acids and potentially cell-free nucleic acids from one or more HPV strains to obtain a plurality of sequence reads; and
determining the amount of HPV fragments based on a total count of the plurality of sequence reads that map to one or more HPV reference genomes corresponding to the one or more HPV strains.

15. The method according to claim 14, wherein the sequencing is performed by whole genome sequencing, targeted sequencing, or whole genome bisulfite sequencing.

16. The method according to claim 13, wherein the cutoff is a count of at least 6 unique HPV fragments, each unique HPV fragment mapping to an HPV reference genome corresponding to at least one HPV strain in the set of HPV strains.

17. The method according to claim 12, wherein the set of HPV strains comprises at least one of HPV 16 or HPV 18.

18. The method according to claim 12, wherein the set of HPV strains includes one or more of HPV 16, 18, 31, 33, 35, 39, 45, 51, 52, 56, 58, 59, 66 and 68.

19. The method according to claim 12, wherein the HPV-based multiclass classifier predicts the scores based on features derived from sequencing the potentially cell-free nucleic acids from the at least one HPV strain in a set of HPV strains in the biological sample, wherein the features comprise one or more of methylation-derived features, a total count of HPV fragments, and a binarized count of HPV fragments.

20. The method according to claim 19, wherein the methylation-derived features comprise features that discriminate pairwise comparisons among HPV-associated cancer types and other cancer types, wherein the other cancer types comprise lung cancers.

21. The method according to claim 19, wherein the sequencing is performed by whole genome sequencing, targeted sequencing, or whole genome bisulfite sequencing.

22. The method according to claim 19, wherein the sequencing is performed by targeted sequencing with a hybridization capture panel containing probes targeting HPV reference genomes corresponding to the set of HPV strains.

23. The method according to claim 22, wherein the probes tile the targeted HPV reference genomes.

24. The method according to claim 12, wherein the plurality of HPV-associated cancer types comprise cervical, anogenital, and head and neck cancers.

25. The method according to claim 12, wherein the HPV-based multiclass classifier comprises a multinomial logistic regression classifier.

26. The method according to claim 12, wherein training of the HPV-based multiclass classifier is restricted to the HPV-positive cancer samples, wherein the HPV-positive cancer samples comprise at least one of cervical, anorectal, and head and neck cancers.

27. The method according to claim 12, comprising:
based on a detection of HPV absence from the biological sample:
forgoing applying the HPV-based multiclass classifier; or
determining an absence of HPV-associated cancer from the biological sample.

28-47. (canceled)

48. A non-transitory computer readable storage medium storing instructions that, when executed by a hardware processor, cause the hardware processor to perform steps comprising:
detecting a presence or absence of HPV in a biological sample comprising cell-free nucleic acids from the subject and potentially cell-free nucleic acids from at least one HPV strain in a set of HPV strains;
based on a detection of HPV viral nucleic acids in the biological sample, applying an HPV-based multiclass classifier that predicts a score for each of a plurality of HPV-associated cancer types, wherein the HPV-based multiclass classifier is trained on a training set comprising HPV-positive cancer samples; and
determining, based on the scores predicted by the HPV multiclass classifier, an HPV-associated cancer associated with the biological sample.

49-55. (canceled)

56. A system for screening for presence of an HPV-associated cancer in a subject, the system comprising a hardware processor and a non-transitory computer-readable storage medium storing instructions that, when executed by the hardware processor, cause the hardware processor to perform steps comprising:
detecting a presence or absence of HPV in a biological sample comprising cell-free nucleic acids from the subject and potentially cell-free nucleic acids from at least one HPV strain in a set of HPV strains;
based on a detection of HPV viral nucleic acids in the biological sample, applying an HPV-based multiclass classifier that predicts a score for each of a plurality of HPV-associated cancer types, wherein the HPV-based multiclass classifier is trained on a training set comprising HPV-positive cancer samples; and
determining, based on the scores predicted by the HPV multiclass classifier, an HPV-associated cancer associated with the biological sample.

57-69. (canceled)
